(19) United States
(12) Patent Application Publication
(10) Pub. No.: US 2026/0048113 A1
(43) Pub. Date: Feb. 19, 2026

(54) ADJUVANTED IMMUNOGENIC COMPOSITION AGAINST NEISSERIA MENINGITIDIS B

(71) Applicant: Sanofi Pasteur Inc., Swiftwater, PA (US)

(72) Inventors: Salvador Fernando Ausar, Toronto (CA); Vinod Balhara, Toronto (CA); Anne-Gaëlle Darmont, Paris (FR); Nausheen Rahman, Toronto (CA)

(73) Assignee: Sanofi Pasteur Inc., Swiftwater, PA (US)

(21) Appl. No.: 19/100,228

(22) PCT Filed: Aug. 2, 2023
(86) PCT No.: PCT/US2023/071474

§ 371 Date: Jan. 31, 2025

Related U.S. Application Data
(60) Provisional application No. 63/370,333, filed on Aug. 3, 2022.

(51) Int. Cl. A61K 39/095; A61K 9/50; A61K 39/00; A61K 39/385; A61K 47/12; A61P 31/04; C07K 14/22

(52) U.S. Cl. CPC A61K 39/095; A61K 9/5068; A61K 39/385; A61K 47/12; A61P 31/04; C07K 14/22; A61K 2039/545; A61K 2039/55505; A61K 2039/5555; A61K 2039/6037

(57) ABSTRACT

The disclosure relates to an immunogenic composition comprising a combination of Neisseria meningitidis serogroup B antigens, said combination comprising at least one factor H binding protein (fHBP) A and at least one factor H binding protein (fHBP) B, and an aluminum hydroxyphosphate (AlPO₄) adjuvant, the AlPO₄ adjuvant being selected as having a point of zero charge (PZC) below 5.

### CROSS-REFERENCE TO RELATED APPLICATIONS

This application claims priority to U.S. Provisional Application No. 63/370,333, filed on Aug. 3, 2022, which is incorporated by reference herein in its entirety for any purpose.

### SEQUENCE LISTING

The present application is filed with a Sequence Listing in electronic format. The Sequence Listing is provided as a file entitled “PR95421_WO_S17_SANOFI_PASTEUR” created on Jul. 21, 2023, which is 14,094 bytes in size. The information in the electronic format of the sequence listing is incorporated herein by reference in its entirety.

### TECHNICAL FIELD

The present disclosure relates to AlPO₄-adjuvanted immunogenic compositions against Neisseria meningitidis B, and methods and uses for stabilizing antigens and enhancing immunogenic responses against autologous and heterologous antigens by means of adjuvants.

### TECHNICAL BACKGROUND

Neisseria meningitidis is a gram-negative diplococcus with humans as the sole known natural host. N. meningitidis is a frequent colonizer of the human naso- and oropharynx but can be found in other areas of the body such as the anal mucosa, the conjunctiva and the urogenital tract (Rouphael et al., Methods Mol Biol. 2012; 799:1-20; Stephens, Vaccine. 2009; 27 Suppl 2:B71-7; Batista et al., Asian Pac J Trop Med. 2017; 10(11):1019-29).

At least 12 different meningococcal serogroups have been classified based on the immunochemistry of the capsular polysaccharides (PS). Some strains are more likely than others to cause infection. Worldwide, most cases of meningococcal disease are caused by serogroups A, B, C, W, X, and Y. Serogroup B is responsible for endemic disease and some outbreaks (Harrison et al., [ed.] Orenstein W A, Offit P A, Edwards K M Plotkin S A. Vaccines. 7. Philadelphia (PA): Elsevier; 2018. p. 619-43; Borrow et al., Expert Rev Vaccines. 2017; 16(4):313-28; Harrison et al., Emerg Infect Dis. 2013; 19(4):566-73, Pollard, Pediatr Infect Dis J. 2004; 23(12 Suppl):S274-9; Kvalsvig et al., J Clin Pathol. 2003; 56(6):417-22).

N. meningitidis serogroup B is a respiratory transmission bacterium (through droplets) that cannot survive in the environment, requiring close and prolonged contact, or direct physical contact (such as a kiss) for effective transmission. The asymptomatic carrier, present among less than 2% of children under 5 years of age and 20%-25% of adolescents and young adults, is the primary element in the pathogen transmission pathway and its maintenance in nature, even during periods of epidemics (Christensen et al., Lancet Infect Dis. 2010; 10(12):853-61; Batista et al., Asian Pac J Trop Med. 2017; 10(11):1019-29).

In general, the age groups with the highest incidence of carriage are adolescents and young adults, who are prone to engage in the behaviors recognized as risk factors for carriage and the eventual appearance of invasive meningococcal disease (IMD) (Christensen et al., Lancet Infect Dis. 2010; 10(12):853-61; Stephens, Vaccine. 2009; 27 Suppl 2:B71-7; Bruce et al., JAMA. 2001; 286(6):688-93; Germinario et al., Hum Vaccin. 2010; 6(12):1025-7; MacLennan et al., Emerg Infect Dis. 2006; 12(6):950-7). Thus, vaccination of these age groups has the potential to impact the incidence of IMD in other age groups, which has been demonstrated in a number of European countries where vaccination campaigns with serogroup C conjugate vaccines have led to herd protection in unvaccinated age groups (Maiden et al., J Infect Dis. 2008; 197(5):737-43; Trotter et al., Lancet. 2004; 364(9431):365-7; Bijlsma et al., Clin Infect Dis. 2014; 59(9):1216-21).

Invasive meningococcal disease (IMD) is a serious illness caused by N. meningitidis (including N. meningitidis serogroup B), and symptoms may include intense headache, fever, nausea, vomiting, photophobia, stiff neck, lethargy, myalgia, and a characteristic petechial rash (Harrison et al., [ed.] Orenstein W A, Offit P A, Edwards K M Plotkin S A. Vaccines. 7. Philadelphia (PA): Elsevier; 2018. p. 619-43). IMD can lead to meningococcal meningoencephalitis and meningococcemia. Meningococcemia is probably the most rapidly fatal infectious condition to humans, with about 90% of deaths reported within the first 2 days of hospitalization. Inflammatory syndromes may arise in 6%-15% of IMD patients, due to deposits of antigen-antibody complex, composed mainly of capsular polysaccharides, specific immunoglobulins and complement fraction C3. These reactions generally occur 4 to 12 days after disease onset and include arthritis, mostly monoarticular (7%-14% of patients), cutaneous vasculitis, iritis, episcleritis, pleuritis and pericarditis. Simultaneously, reappearance of fever, leukocytosis and increased serum C-reactive protein may occur. Other complications that can occur in IMD patients include activation of herpes simplex infection, symmetrical distal necrosis, extensive ulcerations on vasculitis topographies, digestive bleeding, subdural effusion, myocarditis, rhabdomyolysis, adult respiratory distress syndrome, acid-base and hydroelectrolyte disorders, cerebral infarction and intracranial suppuration.

Sequelae may occur in IMD survivors. The risk of neurological sequelae occurrence is 7%-12% (a smaller rate than that of pneumococcal meningitis), primarily occurring in infants. Hearing loss (persistent or transitory) is the most common complication, occurring in approximately 4% of cases. Other sequelae include visual deficits, hydrocephaly, ataxia, dysphasia, motor deficits, developmental delays, arthritis, spasticity, convulsions, renal failure, osteonecrosis, atrophic scarring, loss of parts of the extremities, learning disabilities and behavioral disorders among others (Batista et al., Asian Pac J Trop Med. 2017; 10(11):1019-29; Stephens et al., Neisseria meningitidis. [ed.]J. E. Bennett, R. Dolin and M. J. Blaser. Philadelphia: Elsevier Saunders; 2015. p. 2425-45; Campsall et al., Crit Care Clin. 2013; 29(3):393-409; Pace et al., Vaccine. 2012; 30 Suppl 2:B3-9).

Serogroup B is an important cause of endemic disease and is responsible for multiple prolonged epidemics in several industrialized countries (Vuocolo et al., Hum Vaccin Immunother. 2018; 14(5):1203-15), including Cuba (Rodriguez et al., Mem Inst Oswaldo Cruz. 1999; 94(4):433-40), Norway (Fredriksen et al., NIPH Ann. 1991; 14(2):67-79; discussion-80) and New Zealand (Martin et al., J Infect Dis. 1998; 177(2):497-500; Dyet et al., Epidemiol Infect. 2006; 134(2):377-83). Smaller outbreaks due to a single strain have also been reported in other countries such as France (from 2000-2003) (Grodet et al., Microbiol Infect. 2004; 10(9):845-8; Caron et al., Lancet Infect Dis. 2011; 11(6):455-63) and the United States (from 2013-2017), some of which have been associated with colleges and universities (Folaranmi et al., 2015. MMWR Morb Mortal Wkly Rep. 2015; 64(22):608-12; Atkinson et al., Pharmacotherapy. 2016; 36(8):880-92).

Two vaccines targeting N. meningitidis serogroup B have been recently licensed: the 4-component MenB protein vaccine (4CMenB) BEXSERO® from GlaxoSmithKline, Inc. [GSK], and a bivalent recombinant fHBP protein-based (rLP2086) vaccine (TRUMENBA® from Pfizer). Those vaccines are adjuvanted with, respectively, an aluminum hydroxide adjuvant and an aluminum hydroxyphosphate adjuvant.

Adjuvants are agents incorporated into vaccine formulations to enhance the immunogenicity of vaccine antigens. Aluminum salts, such as aluminum phosphate and aluminum hydroxide, are the most commonly used adjuvants in human and veterinary vaccines today. While a number of aluminum-containing adjuvants are available, for any one specific vaccine formulation to be immunogenic and stable for long term, a proper selection of adjuvant/antigen combination is required.

Aluminum hydroxyphosphate (AlPO₄) adjuvants are amorphous, and most commercially available AlPO₄ adjuvants have a point zero charge (PZC) from 5 to 7 and are either neutral or negatively charged at a neutral pH of 7.0 (Hem S L, 2007). The point zero charge is equivalent to the pI or isoelectric pH of proteins or biomolecules and is defined as the pH at which net charge on the surface of molecule is zero. The point zero charge is dependent on the ratio of hydroxyl and phosphate ions on to the surface of AlPO₄. At neutral pH, AlPO₄ can strongly adsorb proteins with basic pI.

Aluminum hydroxide in its dehydrogenated, crystalline form is chemically aluminum oxyhydroxide [AlO(OH)] and in its aqueous phase, it becomes aluminum trihydroxide [Al(OH)₃] by acquiring an additional water molecule (Stanley L Hem, 2007). Aluminum oxyhydroxide has a PZC of 11, and as such, is positively charged at a neutral pH of 7.0. This positive charge makes aluminum oxyhydroxide a good adsorbent for negatively charged antigens (e.g., acidic pI proteins). The PZC of aluminum hydroxide can be decreased by titrating with phosphate ions. This is caused by the ligand exchange between the hydroxyl ions on the surface of aluminum hydroxide with phosphate ions.

Adsorption of antigens onto aluminum adjuvants is dependent on the physical and chemical characteristics of the antigen, the type of aluminum adjuvant used and the conditions of adsorption. Factors which may affect an antigen's adsorption onto an aluminum adjuvant include electrostatic forces, hydrophobic interactions, Van der Waals forces, hydrogen binding, pH, temperature and size of adjuvant particles. In general, antigens are adsorbed onto aluminum adjuvants through electrostatic attractions (i.e., adjuvant and antigen have opposite charges) and/or through ligand exchanges (i.e., phosphate group on antigen displaces a hydroxyl group on the adjuvant surface) (Seeber S J, 1991; Iyer S, 2004). Electrostatic interactions between the aluminum adjuvant and antigen proteins may be impacted by the pH of the formulation, the point zero charge (PZC) of aluminum, and the isoelectric point (pI) of the proteins. A proper balance of electrostatic interactions is usually sought as proteins too strongly bound to aluminum might elicit a poor immune response and a weak interaction might result in poor adsorption and a lower stability of the product.

The maximum amount of antigen that can be adsorbed as a monolayer to the adjuvant is referred to as the “adsorptive capacity” and the strength of the adsorption is represented by the “adsorptive coefficient” (Jendrek, 2003).

Adsorption may affect a protein's structure and stability. Results from studies on the effect of adsorption to aluminum containing adjuvants are not entirely consistent. In one study, three proteins (bovine serum albumin [BSA], lysozyme and ovalbumin) were destabilized following adsorption onto Alhydrogel® or Adju-Phos®. In another study, the structure of BSA and β-lactoglobulin (BLG) was stabilized by adsorption onto aluminum hydroxide (Jones, 2005; Zheng, 2007). Methods for stabilizing liquid formulations of vaccines with aluminum salt adjuvants include lyophilization, freezing and freeze-drying, but these methods often result in adjuvant agglomeration, decreased immunogen concentration and loss of immunogenicity (Maa, 2003; Diminsky, 1999; Alving, 1993; Warren, 1986). Even for those formulations maintained under refrigerated conditions (e.g., 2° C. to 8° C.) adsorbed antigens may be chemically unstable, and as such, may undergo hydrolysis and fragmentation over time.

WO 2010/109323 discloses immunogenic compositions comprising factor H binding protein (fHBP) antigens adsorbed on aluminum hydroxyphosphate adjuvant. An aluminum hydroxyphosphate adjuvant with a PZC in the range of 5.0 to 7.0 is selected to ensure efficient adsorption of the antigens. Further, the pH of the compositions is selected to be within 1.2 pH units of the PZC.

There is still a need to provide immunogenic compositions comprising an aluminum adjuvant and able to reduce the instability or improve the stability of protein antigens.

There is a need to provide an immunogenic composition comprising aluminum adjuvant and able to induce and enhance an immune response.

There is a need to provide an immunogenic composition comprising aluminum adjuvant offering a good safety.

There is a need to provide an immunogenic composition comprising a combination of Neisseria meningitidis serogroup B antigens and able to induce an enhanced immune response.

There is a need to provide an immunogenic composition comprising a combination of Neisseria meningitidis serogroup B antigens and able to induce an enhanced immune response against heterologous antigens.

There is a need to provide an immunogenic composition comprising a combination of Neisseria meningitidis serogroup B antigens including at least one fHBP A antigen and able to induce an enhanced immune response against fHTBP A antigens heterologous and/or homologous to the fHBP A antigen of the composition.

There is a need to provide an immunogenic composition comprising a combination of Neisseria meningitidis serogroup B antigens including at least one fHBP B antigen and able to induce an enhanced immune response against fHBP B antigens heterologous and/or homologous to the fHBP B antigen of the composition.

There is a need to provide an immunogenic composition comprising a combination of Neisseria meningitidis serogroup B antigens and able to improve the stability of the antigens of the composition.

There is a need to provide an immunogenic composition comprising a combination of Neisseria meningitidis serogroup B antigens including at least a fHTBP A and/or a Neisseria adhesin A (NadA) antigen and able to induce an enhanced stability of the antigens.

The present disclosure aims to satisfy all or part of these needs.

### SUMMARY

According to one of its objects, the present disclosure relates to an immunogenic composition comprising a combination of Neisseria meningitidis serogroup B antigens, said combination comprising at least one factor H binding protein (fHBP) A and at least one factor H binding protein (fHBP) B, and an aluminum hydroxy phosphate (AlPO₄) adjuvant, the AlPO₄ adjuvant being selected as having a point of zero charge (PZC) below 5.

The present disclosure relates to an immunogenic composition comprising a combination of Neisseria meningitidis serogroup B antigens, said combination comprising at least one factor H binding protein (fHBP) A and at least one factor H binding protein (fHBP) B, and an aluminum hydroxyphosphate (AlPO₄) adjuvant, the AIPO4 adjuvant has a PZC below 5 prior to introduction into the composition.

A composition of the disclosure comprises a combination of Neisseria meningitidis serogroup B antigens. The combination comprises at least one factor H binding protein (fHBP) A and at least one factor H binding protein (fHBP) B, and an aluminum hydroxyphosphate (AlPO₄) adjuvant. The AlPO₄ adjuvant is selected as having a “point of zero charge” (PZC) below 5. The fHBP A and B have an isoelectric point (pI) above the PZC of the adjuvant. The pH of the composition is at least 1.2 unit greater than the PZC of the AlPO₄ adjuvant. The composition has a pH from about 5.5 to about 7.0. From about 50% to about 85% of the fHBP A and/or B are adsorbed on the AlPO₄ adjuvant. A composition may further comprise at least one of NadA or dOMV antigen.

In the present disclosure, the AlPO₄ adjuvant has a PZC below 5 prior to introduction in the immunogenic composition.

Surprisingly, by selecting an aluminum hydroxyphosphate (AlPO₄) adjuvant with a PZC below 5—contrary to the recommendations of WO2010/109323—for formulating immunogenic compositions comprising a combination of Neisseria meningitidis serogroup B antigens, including fHBP antigens, the inventors have observed that it was possible to enhance the immune response against the antigens, broaden the N. meningitidis B strains coverage, and further stabilize the antigens.

Surprisingly, the advantageous effects were observed with a pH of the compositions above 1.2 unit from the PZC of the aluminum hydroxyphosphate aluminum.

Surprisingly, the advantageous effects were observed with an adsorption of the fHBP antigens below 85%.

As shown in the Examples section, the selection of an AlPO₄ adjuvant with a PZC below 5 to prepare an immunogenic composition comprising at least one factor H binding protein (fHBP) B allows the composition to induce an enhanced immune response against fHBP B homologous to the fHBP B of the composition (measured with Geometric Mean Titer—GMT—of antibody), compared to a composition prepared with AlPO₄ with a PZC above 5.

Also, as shown in the Examples section, the selection of an AlPO₄ adjuvant having a PZC below 5 to prepare an immunogenic composition comprising at least one factor H binding protein (fHBP) B allows the composition to induce an enhanced immune response against fHBP B heterologous to the fHBP B of the composition (measured with GMT and percentage of responders), compared to a composition prepared with AlPO₄ with a PZC above 5.

Also, as shown in the Examples section, the selection of an AlPO₄ adjuvant having a PZC below 5 to prepare an immunogenic composition comprising at least one factor H binding protein (fHBP) A allows the composition to induce an enhanced immune response against fHBP A homologous to the fHBP A antigen of the composition (measured with GMT and percentage of responders), compared to a composition prepared with AlPO₄ with a PZC above 5.

Also, as shown in the Examples section, the selection of an AlPO₄ adjuvant having a PZC below 5 to prepare an immunogenic composition comprising at least one factor H binding protein (fHBP) A allows the composition to induce an enhanced immune response against fHBP A heterologous to the fHBP A of the composition (measured with GMT and percentage of responders), compared to a composition prepared with AlPO₄ with a PZC above 5.

The enhanced immune response is observed by an increased Geometric Mean Titer (GMT) of functional serum bactericidal antibody activity using human complement (hSBA) obtained with a composition according to the disclosure compared with the hSBA GMT obtained with a composition containing an AlPO₄ with a PZC above 5.

Further, the enhanced immune response is observed by an increased % of responders obtained with a composition according to the disclosure compared with the % of responders obtained with a composition containing an AlPO₄ with a PZC above 5.

An enhanced immune response may be observed by an increased hSBA GMT fHBP specific response and/or an increased % of responders.

Further, as shown in the Examples section, the selection of an AlPO₄ adjuvant having a PZC below 5 to prepare an immunogenic composition according to the disclosure, comprising combination of N. meningitidis serogroup B antigens comprising at least one fHBP A and/or at least one fHBP B advantageously does not impair the stability of the fHBPs.

As shown in the Examples section, the selection of an AlPO₄ adjuvant having a PZC below 5 to prepare an immunogenic composition according to the disclosure, comprising combination of N. meningitidis serogroup B antigens comprising at least one fHBP A allows an enhanced stability of the fHBP A, compared to composition prepared with an AlPO₄ adjuvant with a PZC above 5.

The AlPO₄ adjuvant may be selected to have a PZC ranging from about 4.1 to less than 5, or ranging from about 4.2 to about 4.9, or ranging from about 4.3 to about 4.8, or to be about 4.5.

The AlPO₄ adjuvant may be selected to have a PZC of about 4.5.

The difference between the PZC of the AlPO₄ adjuvant of the disclosure and the pH of the composition is ranging from about 0.6 to about 2.9.

The composition may have a pH from 0.6 to 2.9 units from the PZC of the AlPO₄ adjuvant, or from 1.2 to 2.9 units from the PZC of the adjuvant.

The immunogenic composition may have a pH ranging from about 5.5 to about 7.0, or may have a pH of about 6.0.

An immunogenic composition of the disclosure may further comprise at least one detergent-extracted Outer Membrane Vesicle (dOMV) and/or at least one Neisseria adhesin A (NadA) protein.

As shown in the Examples section, the selection of an AlPO₄ adjuvant having a PZC below 5 to prepare an immunogenic composition according to the disclosure, comprising combination of N. meningitidis serogroup B antigens comprising at least one NadA protein allows an enhanced stability of the NadA protein, compared to composition prepared with AlPO₄ with a PZC above 5.

As shown in the Examples section, the selection of an AlPO₄ adjuvant having a PZC below 5 to prepare an immunogenic composition according to the disclosure, comprising combination of N. meningitidis serogroup B antigens comprising a fHBP A and a NadA protein allows an enhanced stability of the fHBP A and the NadA protein, compared to composition prepared with AlPO₄ with a PZC above 5.

Further, as shown in the Examples section, the selection of an AlPO₄ adjuvant having a PZC below 5 to prepare an immunogenic composition according to the disclosure, comprising combination of N. meningitidis serogroup B antigens comprising at least one NadA protein advantageously does not impair the immune response induced by the NadA protein.

Further, as shown in the Examples section, the selection of an AlPO₄ adjuvant having a PZC below 5 to prepare an immunogenic composition according to the disclosure, comprising combination of N. meningitidis serogroup B antigens comprising at least one detergent-extracted Outer Membrane Vesicle (dOMV) advantageously does not impair the stability or the immune response induced by the dOMV.

Further, as shown in the Examples section, the selection of an AlPO₄ adjuvant having a PZC below 5 to prepare an immunogenic composition according to the disclosure, comprising combination of N. meningitidis serogroup B antigens comprising at least one detergent-extracted Outer Membrane Vesicle (dOMV) advantageously does not increase the pyrogenicity of the composition.

Further, as shown in the Examples section, the selection of an AlPO₄ adjuvant having a PZC below 5 to prepare an immunogenic composition according to the disclosure, comprising combination of N. meningitidis serogroup B antigens comprising at least fHBP A, fHBP B, NadA and dOMV antigens advantageously does not increase the pyrogenicity of the composition.

In an immunogenic composition of the disclosure, the fHBP B may be adsorbed onto AlPO₄ adjuvant in an amount of about 85%, or less, of the total amount of fHBP B present in the composition, or in an amount ranging from about 50% to less than 85% of the total amount of fHBP B present in the composition.

The fHBP B may have an isoelectric point (pI) above the PZC of the AlPO₄ adjuvant.

The fHBP B may have an isoelectric point (pI) ranging from about 5.0 to about 7.0, or from 5.2 to about 6.5, or from about 5.3 to about 6.0, or may be about 5.5, or else 5.46.

The isoelectric point of a fHBP may be determined empirically by a technique such as isoelectric focusing. More conveniently, however, the isoelectric point is a theoretical isoelectric point. This may be calculated using pKa values of the amino acids described in Bjellqvist et al. (1993) Electrophoresis 14:1023-31 using the relevant ExPASy tool (Gasteiger et al. (2005) Protein Identification and Analysis Tools on the ExPASy Server in The Proteomics Protocols Handbook (ed. John M. Walker), Humana Press (2005)).

The fHBP B may be non-lipidated.

The fHBP B may be a mutated fHBP B comprising at least one mutation reducing or suppressing the binding of the fHBP B to the human factor H (fH).

The fHBP B may be a mutated fHBP B comprising at least about 85% identity with SEQ ID NO: 3.

The fHBP B may comprise at least one amino acid substitution selected from at least one of: a) an amino acid substitution of the glutamine at amino acid 38 (Q38); b) an amino acid substitution of the glutamic acid at amino acid 92 (E92); c) an amino acid substitution of the arginine at amino acid 130 (R130); d) an amino acid substitution of the serine at amino acid 223 (S223); and e) an amino acid substitution of the histidine at amino acid 248 (H248), based on the numbering of SEQ ID NO:6, or comprises or consists of SEQ ID NO: 4 or of SEQ ID NO: 9.

In some embodiments, the fHBP B may comprise or consist of SEQ ID NO: 9.

In an immunogenic composition of the disclosure, the fHBP A may be adsorbed onto AlPO₄ adjuvant in an amount of about 85%, or less, of the total amount of fHBP A present in the composition, or in an amount ranging from about 50% to less than 85% of the total amount of fHBP A present in the composition.

The fHBP A may have an isoelectric point (pI) ranging from about 5 to about 7, or from 5.2 to about 6.5, or from about 5.4 to about 6, or is about 5.9, or else 5.86.

The fHBP A may be non-lipidated.

The fHBP A may be a mutated fHBP A comprising at least one mutation reducing or suppressing the binding of the fHBP A to the human factor H (fH).

The fHBP A may be a mutated protein comprising at least about 85% identity with SEQ ID NO: 1.

The fHBP A comprises at least one amino acid substitution selected from at least one of: a) an amino acid substitution of the asparagine at amino acid 115 (N115); b) an amino acid substitution of the aspartic acid at amino acid 121 (D121); c) an amino acid substitution of the serine at amino acid 128 (S128); d) an amino acid substitution of the phenylalanine at amino acid 129 (F129); e) an amino acid substitution of the leucine at amino acid 130 (L130); f) an amino acid substitution of the valine at position 131 (V131); g) an amino acid substitution of the glycine at position 133 (G133); h) an amino acid substitution of the lysine at position 219 (K219); and i) an amino acid substitution of the glycine at position 220 (G220), based on the numbering of SEQ ID NO:6, or comprises or consists of SEQ ID NO: 2 or of SEQ ID NO: 8.

In some embodiments, the fHBP A may comprise or consist of SEQ ID NO: 8.

In an immunogenic composition of the disclosure, the fHBP A and/or the fHBP B may each be present in an amount ranging from about 20 μg/dose to about 200 μg/dose, or from about 25 μg/dose to about 180 μg/dose, or from about 40 μg/dose to about 140 μg/dose, or from about 50 μg/dose to about 120 μg/dose, or from about 75 μg/dose to about 100 μg/dose, or at an amount of about 50 μg/dose, or about 50 μg/dose, or about 100 μg/dose.

A dose may range from about 0.1 mL to about 1 mL, for example from about 0.2 mL to about 0.8 mL, from about 0.4 mL to about 0.6 mL, or may be of about 0.5 mL.

In an immunogenic composition of the disclosure, the fHBP A may be present in an amount ranging from about 20 μg/dose to about 200 μg/dose, or from about 25 μg/dose to about 180 μg/dose, or from about 40 μg/dose to about 140 μg/dose, or from about 50 μg/dose to about 120 μg/dose, or from about 75 μg/dose to about 100 μg/dose, or at an amount of about 50 μg/dose, or about 100 μg/dose.

The fHBP B may be present in an amount ranging from about 20 μg/dose to about 200 μg/dose, or from about 25 μg/dose to about 180 μg/dose, or from about 40 μg/dose to about 140 μg/dose, or from about 50 μg/dose to about 120 μg/dose, or from about 75 μg/dose to about 100 μg/dose, or at an amount of about 50 μg/dose, or about 100 μg/dose.

In an immunogenic composition of the disclosure, the NadA protein may be NadA1 protein or may comprise at least about 85% identity with SEQ ID NO: 5 or comprises or consists of SEQ ID NO:5.

The NadA protein may be present in an amount ranging from about 20 μg/dose to about 200 μg/dose, or from about 25 μg/dose to about 180 μg/dose, or from about 40 μg/dose to about 140 μg/dose, or from about 50 μg/dose to about 120 μg/dose, or from about 75 μg/dose to about 100 μg/dose, or at about 50 μg/dose.

In an immunogenic composition of the disclosure, the dOMV may comprise a porin A (PorA) protein.

The porin A (PorA) protein may be selected among PorA VR2 subtypes or is a PorA VR2 P1.2.

The dOMV may be present in an amount ranging from about 5 μg/dose to about 400 μg/dose, or from about 10 μg/dose to about 300 μg/dose, or from about 25 μg/dose to about 250 μg/dose, or from about 35 μg/dose to about 225 μg/dose, or from about 50 μg/dose to about 200 μg/dose, or from about 75 μg/dose to about 180 μg/dose, or from about 100 μg/dose to about 150 μg/dose, or from about 110 μg/dose to about 125 μg/dose, or at about 25 μg/dose, or at about 50 μg/dose, or at about 125 μg/dose.

An immunogenic composition of the disclosure may further comprise a buffer.

The buffer may be selected among a Tris buffer, an acetate buffer, a citrate buffer, a phosphate buffer, an HEPES buffer, or a histidine buffer.

The buffer may be a sodium acetate buffer.

An immunogenic composition of the disclosure may comprise or consist of 25 to 100 μg/dose of a non-lipidated fHBP A consisting of SEQ ID NO: 2, 25 to 100 μg/dose of a non-lipidated fHBP B consisting of SEQ ID NO: 4, 25 to 100 μg/dose of a NadA protein consisting of SEQ ID NO: 5, 20 to 250 μg/dose of dOMV from a MenB strain expressing PorA VR2 P1.2, 100 to 800 μg/dose of an AlPO₄ adjuvant of PZC of about 4.5, 50 mM of acetate buffer and pH 6.0.

An immunogenic composition of the disclosure may comprise or consist of 25 to 100 μg/dose of a non-lipidated fHBP A consisting of SEQ ID NO: 8, 25 to 100 μg/dose of a non-lipidated fHBP B consisting of SEQ ID NO: 9, 25 to 100 μg/dose of a NadA protein consisting of SEQ ID NO: 5, 20 to 250 μg/dose of dOMV from a MenB strain expressing PorA VR2 P1.2, 100 to 800 μg/dose of an AlPO₄ adjuvant of PZC of about 4.5, 50 mM of acetate buffer and pH 6.0.

An immunogenic composition of the disclosure may further comprise at least a capsular saccharide from one or more of Neisseria meningitidis serogroups A, C, W135 and/or Y conjugated to a carrier protein.

The conjugated capsular saccharide may be conjugated to a tetanus toxoid carrier.

As shown in the Examples section, the selection of an AlPO₄ adjuvant having a PZC below 5 to prepare an immunogenic composition according to the disclosure, comprising combination of N. meningitidis serogroup B antigens and comprising at least one conjugated capsular saccharide from one or more of Neisseria meningitidis serogroups A, C, W135 and/or Y, advantageously does not impair the stability of or the immune response induced by the conjugated capsular saccharide.

An immunogenic composition of the disclosure may have a time of onset of sedimentation (Tₒₙₛₑₜ) ranging from about 3.5 min to about 10 min.

The time of onset sedimentation may be measured as disclosed in the Examples section by the use of static multiple light scattering for detecting particle migration and size variation in liquid dispersions. Two detectors can be used—transmission and backscattering—the signals of which are related to particle size and concentration and their variation is a sign of destabilization that is occurring. This parameter is a measure of the physical stability of a composition and is related to flocculation properties of a suspension. Other methods known in the art may be used to determine time of onset sedimentation.

Advantageously, a time of onset sedimentation, of at least or above 3.5 min allows ensuring that the components of the composition will remain in suspension during the operations for manufacturing, and therefore a better control of the manufacturing.

An immunogenic composition of the disclosure may enhance an immune response against a N. meningitidis serogroup B strain expressing a fHBP B heterologous to the fHBP B of said composition.

An immunogenic composition of the disclosure may enhance an immune response against a N. meningitidis serogroup B strain expressing a fHBP B homologous to the fHBP B of said composition.

An immunogenic composition of the disclosure may enhance an immune response against a N. meningitidis serogroup B strain expressing a fHBP A heterologous to the fHBP A of said composition.

An immunogenic composition of the disclosure may enhance an immune response against a N. meningitidis serogroup B strain expressing a fHBP A homologous to the fHBP A of said composition.

An immunogenic composition of the disclosure may enhance stabilization of a fHBP A.

An immunogenic composition of the disclosure may enhance stabilization of a NadA protein.

According to another of its objects, the present disclosure relates to a vaccine comprising an immunogenic composition of the disclosure.

According to some embodiments, an immunogenic composition or a vaccine of the disclosure may be for use in a method for inducing an immune response against a Neisseria meningitidis B strain.

According to another of its objects, the present disclosure relates to a use of an AlPO₄ adjuvant having a PZC below 5 for enhancing an immune response induced by a composition comprising a N. meningitidis fHBP B antigen against a N. meningitidis serogroup B strain expressing a fHBP B heterologous to said fHBP B antigen of said composition.

According to another of its objects, the present disclosure relates to a use of an AlPO₄ adjuvant having a PZC below 5 for enhancing an immune response induced by a composition comprising a N. meningitidis fHBP B antigen against a N. meningitidis serogroup B strain expressing a fHBP B homologous to said fHBP B antigen of said composition.

According to another of its objects, the present disclosure relates to a use of an AlPO₄ adjuvant having a PZC below 5 for enhancing an immune response induced by a composition comprising a N. meningitidis fHBP A antigen against a N. meningitidis serogroup B strain expressing a fHBP A heterologous to said fHBP A antigen of said composition.

According to another of its objects, the present disclosure relates to a use of an AlPO₄ adjuvant having a PZC below 5 for enhancing an immune response induced by a composition comprising a N. meningitidis fHBP A antigen against a N. meningitidis serogroup B strain expressing a fHBP A homologous to said fHBP A antigen of said composition.

According to another of its objects, the present disclosure relates to a use of an AlPO₄ adjuvant having a PZC below 5 for stabilizing at least one fHBP A in an immunogenic composition.

According to another of its objects, the present disclosure relates to a use of an AlPO₄ adjuvant having a PZC below 5 for stabilizing at least one Neisseria adhesin A (NadA) protein in an immunogenic composition.

According to another of its objects, the present disclosure relates to a use of an AlPO₄ adjuvant having a PZC below 5 for stabilizing a time of onset of sedimentation (Tₒₙₛₑₜ) of a composition comprising a combination of Neisseria meningitidis serogroup B antigens, said combination comprising at least one factor H binding protein (fHBP) A and at least one factor H binding protein (fHBP) B, in a range from about 3.5 min to about 10 min.

According to another of its objects, the present disclosure relates to a use of an AlPO₄ adjuvant having a PZC below 5 for adjuvanting an immunogenic composition comprising a combination of Neisseria meningitidis serogroup B antigens, said combination comprising at least one factor H binding protein (fHBP) A and at least one factor H binding protein (fHBP) B.

According to another of its objects, the present disclosure relates to a use of an AlPO₄ adjuvant having a PZC below 5 for manufacturing an immunogenic composition comprising a combination of Neisseria meningitidis serogroup B antigens, said combination comprising at least one factor H binding protein (fHBP) A and at least one factor H binding protein (fHBP) B.

According to another of its objects, the present disclosure relates to a method for manufacturing an immunogenic composition comprising a combination of Neisseria meningitidis serogroup B antigens, said combination comprising at least one factor H binding protein (fHBP) A and one factor H binding protein (fHBP) B, and an AlPO₄ adjuvant, the method comprising at least the steps of:
a) selecting an AlPO₄ adjuvant having a PZC below 5, and
b) combining the AlPO₄ adjuvant selected at step a) with at least one factor H binding protein (fHBP) A and at least one factor H binding protein (fHBP) B, the combination being carried out in any order.

The combination of the AlPO₄ adjuvant with fHBP A and fHBP B may be carried out in any order. For example, the AlPO₄ adjuvant may be combined with fHBP A, and then fHBP B may be added, or the AlPO₄ adjuvant may be combined with fHBP B, and then fHBP A may be added, or the AlPO₄ adjuvant may be combined with both fHBP A and fHBP B at the same time.

According to another of its objects, the present disclosure relates to a method for stabilizing at least one of fHBP A and NadA protein in an immunogenic composition, the method comprising at least the steps of:
a) selecting an AlPO₄ adjuvant having a PZC below 5, and
b) combining the AlPO₄ adjuvant selected at step a) with a fHBP A or NadA protein, and
c) obtaining an immunogenic composition in which said fHBP A or NadA protein is stabilized.

According to another of its objects, the present disclosure relates to a method for preparing an immunogenic composition comprising a N. meningitidis fHBP B antigen, said composition inducing an enhanced immune response against a N. meningitidis serogroup B strain expressing a fHBP B heterologous to said fHBP B antigen of said composition, the method comprising at least the steps of:
a) selecting an AlPO₄ adjuvant having a PZC below 5, and
b) combining the AlPO₄ adjuvant selected at step a) with said fHBP B antigen, and
c) obtaining said immunogenic composition.

According to another of its objects, the present disclosure relates to a method for preparing an immunogenic composition comprising a N. meningitidis fHBP B antigen, said composition inducing an enhanced immune response against a N. meningitidis serogroup B strain expressing a fHBP B homologous to said fHBP B antigen of said composition, the method comprising at least the steps of:
a) selecting an AlPO₄ adjuvant having a PZC below 5, and
b) combining the AlPO₄ adjuvant selected at step a) with said fHBP B antigen, and
c) obtaining said immunogenic composition.

The composition may further comprise at least one of a fHBP A, NadA protein or dOMV.

According to another of its objects, the present disclosure relates to a method for preparing an immunogenic composition comprising a N. meningitidis fHBP A antigen, said composition inducing an enhanced immune response against a N. meningitidis serogroup B strain expressing a fHBP A heterologous to said fHBP A antigen of said composition, the method comprising at least the steps of:
a) selecting an AlPO₄ adjuvant having a PZC below 5, and
b) combining the AlPO₄ adjuvant selected at step a) with said fHBP A antigen, and
c) obtaining said immunogenic composition.

According to another of its objects, the present disclosure relates to a method for preparing an immunogenic composition comprising a N. meningitidis fHBP A antigen, said composition inducing an enhanced immune response against a N. meningitidis serogroup B strain expressing a fHBP A homologous to said fHBP A antigen of said composition, the method comprising at least the steps of.
a) selecting an AlPO₄ adjuvant having a PZC below 5, and
b) combining the AlPO₄ adjuvant selected at step a) with said fHBP A antigen, and
c) obtaining said immunogenic composition.

The composition may further comprise at least one of a fHBP B, NadA protein or dOMV.

According to another of its objects, the present disclosure relates to a method for inducing an immune response against a Neisseria meningitidis serogroup B strain in an individual in need thereof, the method comprising at least a step of administering to said individual an immunogenic composition or a vaccine according to the disclosure, wherein said step of administration induces an immune response against said Neisseria meningitidis serogroup B strain.

A method for enhancing an immune response induced by a composition comprising a N. meningitidis fHBP B antigen against a N. meningitidis serogroup B strain expressing a fHBP B heterologous to said fHBP B antigen of said composition, in an individual in need thereof, the method comprising at least a step of administering to said individual an immunogenic composition or a vaccine according to the disclosure, wherein said step of administration induces an enhanced immune response against said Neisseria meningitidis serogroup B strain expressing said heterologous fHBP B.

A method for enhancing an immune response induced by a composition comprising a N. meningitidis fHBP B antigen against a N. meningitidis serogroup B strain expressing a fHBP B homologous to said fHBP B antigen of said composition, in an individual in need thereof, the method comprising at least a step of administering to said individual an immunogenic composition or a vaccine according to the disclosure, wherein said step of administration induces an enhanced immune response against said Neisseria meningitidis serogroup B strain expressing said homologous fHBP B.

A method for enhancing an immune response induced by a composition comprising a N. meningitidis fHBP A antigen against a N. meningitidis serogroup B strain expressing a fHBP A heterologous to said fHBP A antigen of said composition, in an individual in need thereof, the method comprising at least a step of administering to said individual an immunogenic composition or a vaccine according to the disclosure, wherein said step of administration induces an enhanced immune response against said Neisseria meningitidis serogroup B strain expressing said heterologous fHBP A.

A method for enhancing an immune response induced by a composition comprising a N. meningitidis fHBP A antigen against a N. meningitidis serogroup B strain expressing a fHBP A homologous to said fHBP A antigen of said composition, in an individual in need thereof, the method comprising at least a step of administering to said individual an immunogenic composition or a vaccine according to the disclosure, wherein said step of administration induces an enhanced immune response against said Neisseria meningitidis serogroup B strain expressing said homologous fHBP A.

### DESCRIPTION OF THE FIGURES

FIG. 1: shows results of the hSBA measured against A05 closely-related (also called homologous) fHBP A56 expressing Neisseria meningitidis strain in sera (purified IgG) collected on D0 (Grey) and D42 (Black) from rabbits immunized on D0 and D28 with MenB immunogenic compositions formulated with AlPO₄ adjuvant (PZC 5.2) or mod-AlPO₄ adjuvant (PZC 4.5).

FIG. 2: shows results of the hSBA measured against B01 closely-related (homologous) fHBP B44 expressing Neisseria meningitidis strain in sera (purified IgG) collected on D0 (Grey) and D42 (Black) from rabbits immunized on D0 and D28 with MenB immunogenic compositions formulated with AlPO₄ adjuvant (PZC 5.2) or mod-AlPO₄ adjuvant (PZC 4.5).

FIG. 3: shows results of the hSBA measured against A05 heterologous fHBP A22 expressing Neisseria meningitidis strain in sera (purified IgG) collected on D0 (Grey) and D42 (Black) from rabbits Immunized on D0 and D28 with MenB immunogenic compositions formulated with AlPO₄ adjuvant (PZC 5.2) or mod-AlPO₄ adjuvant (PZC 4.5).

FIG. 4: shows results of the measured against B01 heterologous fHBP B24 expressing Neisseria meningitidis strain in sera (purified IgG) collected on D0 (Grey) and D42 (Black) from rabbits immunized on D0 and D28 with MenB immunogenic compositions formulated with AlPO₄ adjuvant (PZC 5.2) or mod-AlPO₄ adjuvant (PZC 4.5).

FIG. 5: shows results of the hSBA measured against VR2-P1.2-PorA expressing Neisseria meningitidis strain in sera (purified IgG) Collected on D0 (Grey) and D42 (Black) from rabbits immunized on D0 and D28 with MenB immunogenic compositions formulated with AlPO₄ adjuvant (PZC 5.2) or mod-AlPO₄ adjuvant (PZC 4.5).

FIG. 6: shows results of the hSBA measured against NadA expressing Neisseria meningitidis strain in sera (purified IgG) collected on D0 (Grey) and D42 (Black) from rabbits immunized on D0 and D28 with MenB immunogenic compositions formulated with AlPO₄ adjuvant (PZC 5.2) or mod-AlPO₄ adjuvant (PZC 4.5).

FIG. 7: shows results of the hSBA measured against Neisseria meningitidis A strain in sera (purified IgG) collected on D0, D28 and D42 from rabbits immunized on D0 and D28 with MenACWY immunogenic composition formulated without AlPO₄ adjuvant (white), or with mod-AlPO₄ adjuvant (PZC 4.5) (patterned), or with MenB antigen and mod-AlPO₄ adjuvant (PZC 4.5) (black).

FIG. 8: shows results of the hSBA measured against Neisseria meningitidis C strain in sera (purified IgG) collected on D0, D28 and D42 from rabbits immunized on D0 and D28 with MenACWY immunogenic composition formulated without AlPO₄ adjuvant (white), or with mod-AlPO₄ adjuvant (PZC 4.5) (patterned), or with MenB antigen and mod-AlPO₄ adjuvant (PZC 4.5) (black).

FIG. 9: shows results of the hSBA measured against Neisseria meningitidis W135 strain in sera (purified IgG) collected on D0, D28 and D42 from rabbits immunized on D0 and D28 with MenACWY immunogenic composition formulated without AlPO₄ adjuvant (white), or with mod-AlPO₄ adjuvant (PZC 4.5) (patterned), or with MenB antigen and mod-AlPO₄ adjuvant (PZC 4.5) (black).

FIG. 10: shows results of the hSBA measured against Neisseria meningitidis Y strain in sera (purified IgG) collected on D0, D28 and D42 from rabbits immunized on D0 and D28 with MenACWY immunogenic composition formulated without AlPO₄ adjuvant (white), or with mod-AlPO₄ adjuvant (PZC 4.5) (patterned), or with MenB antigen and mod-AlPO₄ adjuvant (PZC 4.5) (black).

FIGS. 11A-11C: shows results of the relative antigenicity (RA) of A05tmN, B01smN, NadA and dOMV formulated in a MenPenta immunogenic composition with AlPO₄ adjuvant (PZC 5.2) or mod-AlPO₄ adjuvant (PZC 4.5) and submitted at a thermal stress of 45° C. (or 37° C. for NadA) for 20 days.

FIGS. 12A-12D: shows the percentage of free polysaccharide changes for serogroups A, C, W-135 and Y formulated in a MenPenta immunogenic composition with AlPO₄ adjuvant (PZC 5.2) or mod-AlPO₄ adjuvant (PZC 4.5) and submitted to a temperature of 5° C. or to a thermal stress of 45° C. for 28 days.

FIGS. 13A-13D: shows the stability of A05tmN, B01smN, NadA and dOMV in AlPO₄ adjuvants of different points of zero charge (PZC), AlPO₄ adjuvant (PZC 5.2) or mod-AlPO₄ adjuvant (PZC of 4.3, 4.5 or 4.8), at 45° C. for B01, A05 and dOMV or 37° C. for NadA, for 30 days, expressed as the relative antigenicity (RA) of A05tmN, B01smN, NadA or dOMV.

### DESCRIPTION OF THE SEQUENCES

SEQ ID NO: 1 represents fHTBP A05 wild-type sequence without the 19 first N-terminal amino acids corresponding to the signal peptide responsible for lipidation.

CSSGSGSGGGGVAADIGTGLADALTAPLDHKDKGLKSLTLEDSISQNGT
LTLSAQGAEKTFKVGDKDNSLNTGKLKNDKISRFDFVQKIEVDGQTITL
ASGEFQIYKQDHSAVVALQIEKINNPDKIDSLINQRSFLVSGLGGEHTA
FNQLPSGKAEYHGKAFSSDDAGGKLTYTIDFAAKQGHGKIEHLKTPEQN
VELASAELKADEKSHAVILGDTRYGSEEKGTYHLALFGDRAQEIAGSAT
VKIREKVHEIGIAGKQ

SEQ ID NO: 2 represents the mutated fHBP A05 sequence without the signal peptide responsible for lipidation and with the mutations G220S, L130R, G133D (numbering is determined with respect to sequence SEQ ID NO: 6 (fHBP B24)).

CSSGSGSGGGGVAADIGTGLADALTAPLDHKDKGLKSLTLEDSISQNGT
LTLSAQGAEKTFKVGDKDNSLNTGKLKNDKISRFDFVQKIEVDGQTITL
ASGEFQIYKQDHSAVVALQIEKINNPDKIDSLINQRSFRVSDLGGEHTA
FNQLPSGKAEYHGKAFSSDDAGGKLTYTIDFAAKQGHGKIEHLKTPEQN
VELASAELKADEKSHAVILGDTRYGSEEKSTYHLALFGDRAQEIAGSAT
VKIREKVHEIGIAGKQ

SEQ ID NO: 3 represents fHBP B01 wild-type sequence without the signal peptide responsible for lipidation.

CSSGGGGSGGGGVTADIGTGLADALTAPLDHKDKGLKSLTLEDSISQNG
TLTLSAQGAEKTYGNGDSLNTGKLKNDKVSRFDFIRQIEVDGQLITLES
GEFQVYKQSHSALTALQTEQEQDPEHSEKMVAKRRFRIGDIAGEHTSFD
KLPKDVMATYRGTAFGSDDAGGKLTYTIDFAAKQGHGKIEHLKSPELNV
DLAVAYIKPDEKHHAVISGSVLYNQDEKGSYSLGIFGEKAQEVAGSAEV
ETANGIHHIGLAAKQ

SEQ ID NO: 4 represents the mutated fHBP B01 sequence without the signal peptide responsible for lipidation and with the mutation H248L (numbering is determined with respect to sequence SEQ ID NO: 6 (fHBP B24)).

CSSGGGGSGGGGVTADIGTGLADALTAPLDHKDKGLKSLTLEDSISQNG
TLTLSAQGAEKTYGNGDSLNTGKLKNDKVSRFDFIRQIEVDGQLITLES
GEFQVYKQSHSALTALQTEQEQDPEHSEKMVAKRRFRIGDIAGEHTSFD
KLPKDVMATYRGTAFGSDDAGGKLTYTIDFAAKQGHGKIEHLKSPELNV
DLAVAYIKPDEKHHAVISGSVLYNQDEKGSYSLGIFGEKAQEVAGSAEV
ETANGIHLIGLAAKQ

SEQ ID NO: 5 represents NadA1 sequence from MenB MC58 strain in which the 23 amino acids of the signal peptide in N-terminus and the last 55 amino acids of the C-terminus have been deleted.

MTSDDDVKKAATVAIVAAYNNGQEINGFKAGETIYDIGEDGTITQKDAT
AADVEADDFKGLGLKKVVTNLTKTVNENKQNVDAKVKAAESEIEKLTTK
LADTDAALADTDAALDETTNALNKLGENITTFAEETKTNIVKIDEKLEA
VADTVDKHAEAFNDIADSLDETNTKADEAVKTANEAKQTAEETKQNVDA
KVKAAETAAGKAEAAAGTANTAADKAEAVAAKVTDIKADIATNKADIAK
NSARIDSLDKNVANLRKETRQGLAEQAALSGLFQPYNVG

SEQ ID NO: 6 represents the fHBP B24 wild-type sequence on the basis of which the numbering of the positions of the mutations in A05 and B01 are determined.

CSSGGGGVAADIGAGLADALTAPLDHKDKGLQSLTLDQSVRKNEKLKLA
AQGAEKTYGNGDSLNTGKLKNDKVSRFDFIRQIEVDGQLITLESGEFQV
YKQSHSALTAFQTEQIQDSEHSGKMVAKRQFRIGDIAGEHTSFDKLPEG
GRATYRGTAFGSDDAGGKLTYTIDFAAKQGNGKIEHLKSPELNVDLAAA
DIKPDGKRHAVISGSVLYNQAEKGSYSLGIFGGKAQEVAGSAEVKTVNG
IRHIGLAAKQ

SEQ ID NO: 7 represents the wild-type NadA1 sequence from MenB MC58 strain.

MKHFPSKVLTTAILATFCSGALAATSDDDVKKAATVAIVAAYNNGQEIN
GFKAGETIYDIGEDGTITQKDATAADVEADDFKGLGLKKVVTNLTKTVN
ENKQNVDAKVKAAESEIEKLTTKLADTDAALADTDAALDETTNALNKLG
ENITTFAEETKTNIVKIDEKLEAVADTVDKHAEAFNDIADSLDETNTKA
DEAVKTANEAKQTAEETKQNVDAKVKAAETAAGKAEAAAGTANTAADKA
EAVAAKVTDIKADIATNKADIAKNSARIDSLDKNVANLRKETRQGLAEQ
AALSGLFQPYNVGRFNVTAAVGGYKSESAVAIGTGFRFTENFAAKAGVA
VGTSSGSSAAYHVGVNYEW

SEQ ID NO: 8 represents the non-lipidated mutated fHBP A05 sequence with the mutations G220S, L130R, G133D (numbering is determined with respect to sequence SEQ ID NO: 6 (fHBP B24)) and with the substitution of the N-terminal cysteine by a methionine by fusing the ATG start codon directly to the second 5′ terminal codon in the DNA sequence coding for the recombinant protein.

MSSGSGSGGGGVAADIGTGLADALTAPLDHKDKGLKSLTLEDSISQNGT
LTLSAQGAEKTFKVGDKDNSLNTGKLKNDKISRFDFVQKIEVDGQTITL
ASGEFQIYKQDHSAVVALQIEKINNPDKIDSLINQRSFRVSDLGGEHTA
FNQLPSGKAEYHGKAFSSDDAGGKLTYTIDFAAKQGHGKIEHLKTPEQN
VELASAELKADEKSHAVILGDTRYGSEEKSTYHLALFGDRAQEIAGSAT
VKIREKVHEIGIAGKQ

SEQ ID NO: 9 represents the non-lipidated mutated fHBP B01 sequence with the mutation H248L (numbering is determined with respect to sequence SEQ ID NO: 6 (fHBP B24)) and with the substitution of the N-terminal cysteine by a methionine by fusing the ATG start codon directly to the second 5′ terminal codon in the DNA sequence coding for the recombinant protein.

MSSGGGGSGGGGVTADIGTGLADALTAPLDHKDKGLKSLTLEDSISQNG
TLTLSAQGAEKTYGNGDSLNTGKLKNDKVSRFDFIRQIEVDGQLITLES
GEFQVYKQSHSALTALQTEQEQDPEHSEKMVAKRRFRIGDIAGEHTSFD
KLPKDVMATYRGTAFGSDDAGGKLTYTIDFAAKQGHGKIEHLKSPELNV
DLAVAYIKPDEKHHAVISGSVLYNQDEKGSYSLGIFGEKAQEVAGSAEV
ETANGIHLIGLAAKQ

### DETAILED DESCRIPTION

### Definitions

Unless otherwise defined herein, scientific and technical terms used in connection with the present invention shall have the meanings that are commonly understood by those of ordinary skill in the art. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Methods are performed according to kits manufacturer's specifications, as commonly accomplished in the art or as described herein. Further, unless otherwise required by context, singular terms shall include pluralities and plural terms shall include the singular.

Units, prefixes, and symbols are denoted in their Systeme International d'Unités (SI) accepted form. Numeric ranges are inclusive of the numbers defining the range. Unless otherwise indicated, amino acid sequences are written left to right in amino (N-) to carboxyl (-C) orientation. The headings provided herein are not limitations of the various aspects of the disclosure. Accordingly, the terms defined immediately below are more fully defined by reference to the specification in its entirety.

All publications and other references mentioned herein are incorporated by reference in their entirety. Although a number of documents are cited herein, this citation does not constitute an admission that any of these documents forms part of the common general knowledge in the art.

It is to be noted that as used herein and in the appended claims, the singular forms “a”, “an”, and “the” include plural referents unless the context clearly dictates otherwise. Thus, for example, reference to “an antigen” includes a plurality of such antigens and reference to “the protein” includes reference to one or more proteins, and so forth.

It is understood that aspects and embodiments of the present disclosure described herein include “having,” “comprising,” “consisting of,” and “consisting essentially of” aspects and embodiments. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the basic and novel characteristic(s) of the disclosure. It is understood that the different embodiments of the disclosure using the term “comprising” or equivalent cover the embodiments where this term is replaced with “consisting of” or “consisting essentially of”.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, 0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

Within the disclosure, the term “significantly” used with respect to change intends to mean that the observe change is noticeable and/or it has a statistic meaning.

Within the disclosure, the term “substantially” used in conjunction with a feature of the disclosure intends to define a set of embodiments related to this feature which are largely but not wholly similar to this feature.

Within the disclosure, “immunogenic composition” intends to refer to a composition comprising at least one antigen in a sufficient amount and in a suitable formulation for inducing an immune response directed towards said antigen in an individual to which the composition is administered. The immune response may a humoral and/or a cellular response.

Within the disclosure, “PZC” intends to mean point of zero charge and intends to refer to the pH at which the net surface charge of adsorbent is equal to zero.

Within the disclosure, “pI” intends to mean isoelectric point and intends to refer to the pH at which a particular molecule carries no net electrical charge.

Within the disclosure, the term “antigen” comprises any molecule, for example a peptide or protein, which comprises at least one epitope that will elicit an immune response and/or against which an immune response is directed. For example, an antigen is a molecule which, optionally after processing, induces an immune response, which is for example specific for the antigen or cells expressing the antigen. After processing, an antigen may be presented by MHC molecules and reacts specifically with T lymphocytes (T cells). According to the present disclosure, any suitable antigen may be envisioned which is a candidate for an immune response. An antigen may correspond to or may be derived from a naturally occurring antigen.

Within the disclosure, the term “adjuvant” intends to refer to a compound able to enhance an immune response directed against an antigen, including enhancing the magnitude and/or duration of an immune response generated by an antigen.

Within the disclosure, the term “buffer” intends to refer to an aqueous solution containing either a weak acid and its salt or a weak base and its salt, and which is resistant to changes in pH. Buffers are used to maintain a stable pH in a solution, as they can neutralize small quantities of additional acid of base. Buffers suitable for immunogenic compositions are known in the art.

Herein the expression “immune response” intends to refer to the biological reactions occurring in in a subject in which the body recognizes and defends itself against antigens, i.e., bacteria, viruses, and substances that appear foreign and harmful. An immune response may have a humoral, i.e., antibodies, or a cellular component.

Herein, the expression “enhancing an immune response” intends to refer to an immune response, measured in its humoral and/or cellular components, induced by a first immunogenic composition which is greater that the immune response, similarly measured, induced by a second immunogenic composition, the first and second composition being different by one parameter. The difference may be a trend or statistically significant. Within the present disclosure, to determine an enhancement of an immune response, a composition of the disclosure is compared with a composition containing an AlPO₄ adjuvant different from the AlPO₄ of the disclosure, the other parameters of the composition being elsewhere identical.

Herein, the terms “stabilize”, “stabilizing” or “stabilization” used with regard to an antigen intends to refer to the maintenance, over a certain period of time, at a given or unfluctuating level, of the immunogenic property of this antigen. The stabilizing effect of a formulation with regard to an antigen may typically be shown by the reduction or absence of loss of immunogenicity or potency of the antigen during stresses—physical, chemical or mechanical (for instance shift in pH, changes in temperature, surface interactions, extraneous impurities, mixing, etc.) in presence of the formulation compared to the loss of immunogenicity of the antigen in absence of the formulation or in presence of another formulation. The immunogenicity or potency may be measured at one point in time, or repeatedly over a certain period of time, for example, 2, 3, 4, 6 or 8 times over a period of time of 3, 6, 9 or 12 months. Immunogenicity (or potency) of an antigen is its capacity to induce an immune response and can be measured by any known method in the art.

Instability of a protein antigen may be caused by chemical degradation or aggregation of the molecules to form higher order polymers, by dissociation of the heterodimers into monomers, deglycosylation, modification of glycosylation, or any other structural modification that reduces at least one biological activity of the antigen.

As used herein, the term “vaccine” is intended to mean an immunogenic composition directed to a pathogenic agent which is administered to a subject to induce an immune response with the intent to protect the subject from an illness caused by the pathogenic agent (i.e., to confer a protective immunity) or treat an illness caused by the pathogenic agent. A vaccine as disclosed herein may be used as a preventive (prophylactic) vaccine, for administration to a subject prior to infection, with the intent to prevent, or reduced the likelihood of occurrence of, initial (and/or recurrent) infection.

In the context of the disclosure, the term “protective immunity” means that a vaccine or immunization schedule that is administered to a mammal induces an immune response that prevents, retards the development of, or reduces the severity of a disease that is caused by Neisseria meningitidis, or diminishes or altogether eliminates the symptoms of the disease. Protective immunity can be accompanied by production of bactericidal antibodies. It should be noted that production of bactericidal antibodies against Neisseria meningitidis is accepted in the field as predictive of a vaccine's protective effect in humans. (Goldschneider et al. (1969) J. Exp. Med. 129:1307).

An “isolated” protein or a fragment, variant, or derivative thereof refers to a protein that is not in its natural milieu. No particular level of purification is required. For example, an isolated protein can simply be removed from its native or natural environment. Recombinantly produced proteins expressed in host cells are considered isolated for the purpose of the disclosure, as are native or recombinant polypeptides which have been separated, fractionated, or partially or substantially purified by any suitable technique.

As used herein, the terms “individual” or “subject” or “patient” are used interchangeably and are intended to refer to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some exemplary embodiments, the individual or subject is a human.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention. All publications mentioned herein are incorporated herein by reference to disclose and describe the methods and/or materials in connection with which the publications are cited.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of the list of items and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

### Aluminium Hydroxyphosphate (AlPO₄) Adjuvant

Compositions of the disclosure include an aluminium hydroxyphosphate adjuvant.

Aluminium hydroxyphosphate adjuvant, represented by the chemical formula Al(OH)PO₄, and referred in the description as AlPO₄ adjuvant, is not a stoichiometric compound and the amounts of hydroxyl and phosphate moieties depend on conditions of preparation. The respective proportion of hydroxyl and phosphate moieties affects the adjuvant's point of zero charge (PZC).

The PZC corresponds to the pH at which a surface has no net charge. The PZC is inversely related to the degree of substitution of phosphate for hydroxyl (the P/Al molar ratio). Substitution of phosphate anions for hydroxyl anions lowers the PZC. The PZC may be altered by changing the concentration of free phosphate ions in solution (more phosphate=more acidic, or lower, PZC) or by adding a buffer such as a histidine buffer (this makes PZC more basic, or higher).

The AlPO₄ adjuvant used within the disclosure is selected as having a PZC below 5. The PZC is measured before introduction of the AlPO₄ adjuvant into the composition. Therefore, an AlPO₄ adjuvant suitable for the disclosure is selected on the basis of its PZC below 5 prior to its introduction into the immunogenic composition. A selected AlPO₄ has therefore a PZC below 5 prior its introduction into the composition.

The AlPO₄ adjuvant may be selected to have a PZC ranging from about 4.1 to less than 5, or ranging from about 4.2 to about 4.9, or ranging from about 4.3 to about 4.8, or ranging from about 4.4 to about 4.6, or to be about 4.5.

The AlPO₄ adjuvant may be selected to have a PZC of about 4.1, 4.2, 4.3, 4.4, 4.5, 4.6, 4.7, 4.8, or 4.9.

The AlPO₄ adjuvant may be selected to have a PZC of about 4.3, 4.5 or 4.8.

The AlPO₄ adjuvant may be selected to have a PZC of about 4.5.

The AlPO₄ adjuvant may be selected to have a PZC of 4.5.

Different methods can be used to obtain AlPO₄ adjuvant with a target PZC.

To prepare an AlPO₄ adjuvant with a target PZC, one may use an aluminum hydroxide (Al(OH)₃) adjuvant or aluminum hydroxyphosphate adjuvant with a PZC higher than the target PZC. Such AlPO₄ adjuvants are commercially available.

In one method, a phosphate buffer pH 5.8 formulated by a combination of 0.5 M sodium phosphate monobasic and 0.5 M sodium phosphate dibasic can be added to aluminum hydroxide or to an aluminum hydroxyphosphate adjuvant with a higher PZC to make an AlPO₄ adjuvant with a target PZC.

In another method, the PZC of an AlPO₄ adjuvant may be modified by titrating an AlPO₄ adjuvant with a higher PZC with a 0.5 M stock solution of sodium phosphate monobasic salt.

The AlPO₄ adjuvant may be prepared by a batch precipitation method with three reactants: aluminum chloride (or other source of aluminum), trisodium sodium phosphate and a sodium hydroxide.

The P/Al molar ratio of an aluminium hydroxyphosphate adjuvant will generally be between 0.3 and 1.2, preferably between 0.8 and 1.2, or between 0.85 and 1.0, and more preferably about 0.9. A P/Al molar ratio of at least 0.5 can provide an adjuvant with better immunogenicity and stability properties.

AlPO₄ adjuvants are generally amorphous (i.e., amorphous to X-rays). They are generally particulate (e.g., they exhibit plate-like morphology as seen in transmission electron micrographs). Typical diameters of the plates may be 10-100 nm, and these form aggregates sized 0.5-20 μm (e.g., about 1-10 μm). Adsorptive capacities of between 0.7-1.5 mg protein per mg Al³⁺ at pH 7.4 have been reported for aluminium hydroxyphosphate adjuvants.

A typical adjuvant is amorphous aluminium hydroxyphosphate with P/Al molar ratio between 0.84 and 0.92, and this adjuvant may be included at 0.8 mg Al³⁺/mL.

The concentration of Al (Al³⁺) may be preferably less than 5 mg/mL e.g., <4 mg/mL, <3 mg/mL, <2 mg/mL, <1 mg/mL, etc. A suitable range may be from about 0.2 to about 1 mg/mL, or from 0.2 to about 0.8 mg/mL. An Al concentration of 0.8 mg/dose may be used.

An aluminum phosphate adjuvant may be present in a composition disclosed herein in an amount ranging from about 100 μg/dose to about 1000 μg/dose, or from about 150 μg/dose to about 900 μg/dose, or from about 200 μg/dose to about 800 μg/dose, or from about 250 μg/dose to about 700 μg/dose, or from about 300 μg/dose to about 600 μg/dose, or from about 350 μg/dose to about 550 μg/dose, or from about 400 μg/dose to about 500 μg/dose, or from about 400 μg/dose to about 800 μg/dose, or at about 400 μg/dose, or at about 800 μg/dose.

An aluminum phosphate adjuvant may be present in a composition disclosed herein in an amount of about 400 μg/dose.

In compositions of the disclosure, the fHBPs may be adsorbed onto AlPO₄ adjuvant at an amount of about 85%, or less, of the total amount of fHBPs of the composition, or at an amount ranging from about 50% to less than 85%, or about 70% to about 80% of the total amount of fHBPs of the composition. The fHBPs may be adsorbed on AlPO₄ adjuvant at an amount ranging from about 50 to about 85%, or less than 85%, or from about 50 to about 80%, or from about 50 to about 75%, or from about 65 to about 75% of the total amount of fHBPs of the composition.

In an immunogenic composition of the disclosure, the fHBP B may be adsorbed onto the AlPO₄ adjuvant in an amount of about 85%, or less, of the total amount of fHBP B present in the composition, or in an amount ranging from about 50% to less than 85% of the total amount of fHBP B present in the composition. The fHBP B may be adsorbed onto the AlPO₄ adjuvant at an amount ranging from about 50 to about 85%, or less than 85%, or from about 50 to about 80%, or from about 50 to about 75%, or from about 65 to about 75%, of the total amount of fHBP B of the composition. The fHBP B may be adsorbed onto the AlPO₄ adjuvant at an amount of about 50, 55, 60, 65, 70, 75 or 80%, or less than 85% of the total amount of fHBP B present in the composition.

In an immunogenic composition of the disclosure, the fHBP A may be adsorbed onto the AlPO₄ adjuvant in an amount of about 85%, or less, of the total amount of fHBP A present in the composition, or in an amount ranging from about 50% to less than 85% of the total amount of fHBP A present in the composition. The fHBP A may be adsorbed onto the AlPO₄ adjuvant at an amount ranging from about 50 to about 85%, or less than 85%, or from about 50 to about 80%, or from about 50 to about 75%, or from about 65 to about 75% of the total amount of fHBP A of the composition. The fHBP A may be adsorbed onto the AlPO₄ adjuvant at an amount of about 50, 55, 60, 65, 70, 75 80%, or less than 85% of the total amount of fHBP A present in the composition.

The proportion of adsorbed fHBP can be controlled by altering salt concentration and/or pH during formulation e.g., in general, a higher NaCl concentration can decrease fHBP adsorption onto the AlPO₄ adjuvant. The amount of adsorption for any formulation will depend on a combination of parameters including the adjuvant's PZC, the salt concentration and pH during formulation, the adjuvant concentration, the antigen concentration and the antigen's pI. The impact of each of these parameters on adsorption can be readily assessed. The degree of adsorption can be determined by comparing the total amount of fHBP antigen in a composition (e.g., measured before adsorption occurs, or measured by desorbing adsorbed antigen) to the amount which remains in the supernatant after centrifugation. A suitable method may be the method disclosed in the Example section.

In some embodiments, AlPO₄ adjuvants of the disclosure are used in compositions having a pH ranging from about 5.5 to about 7.0. The compositions of the disclosure may have a pH of about 6.0.

In some embodiments, AlPO₄ adjuvants of the disclosure are used in compositions having a pH such that the difference between the PZC of the AlPO₄ adjuvant and a pH of the composition is ranging from about 0.6 to about 2.9.

The compositions may have a pH from 0.6 to 2.9 units from the PZC of the AlPO₄ adjuvant, or from 1.0 to 2.8, or from 1.2 to 2.5, or from 1.4 to 2.1 units from the PZC of the adjuvant.

The compositions may have a pH of at least 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, or 2.9 units from the PZC of the AlPO₄ adjuvant.

In some embodiments, AlPO₄ adjuvants of the disclosure are used in compositions having a pH such that the difference between the PZC of the AlPO₄ adjuvant and the pH of the composition is ranging from about 1.0 to about 2.9, or from about 1.2 to about 2.9.

The compositions may have a pH of at least 1.2 units from the PZC of the AlPO₄ adjuvant.

The compositions may have a pH of at no more 2.9 units from the PZC of the AlPO₄ adjuvant.

The compositions may have a pH of 1.2, 1.3, 1.4, 1.5, 1.6, 1.7, 1.8, 1.9, 2.0, 2.1, 2.2, 2.3, 2.4, 2.5, 2.6, 2.7, 2.8, or 2.9 units from the PZC of the AlPO₄ adjuvant.

The compositions may have a pH of 1.2, 1.3, 1.4, 1.5, 1.6, 1.7, 1.8, 1.9, 2.0, 2.1, 2.2, 2.3, 2.4, 2.5, 2.6, or 2.7 units from the PZC of the AlPO₄ adjuvant.

The compositions may have a pH of 1.2, 1.5, 1.7, 2.2, 2.5 or 2.7 units from the PZC of the AlPO₄ adjuvant.

The compositions may have a pH of 1.2, 1.3, 1.4, 1.5, 1.6 or 1.7 units from the PZC of the AlPO₄ adjuvant.

The compositions may have a pH of 1.2, 1.5 or 1.7 units from the PZC of the AlPO₄ adjuvant.

The compositions may have a pH from about 5.5 to about 7.0. The compositions may have a pH of about 5.5, 5.6, 5.7, 5.8, 5.9, 6.0, 6.1, 6.2, 6.3, 6.4, 6.5, 6.6, 6.7, 6.8, 6.9, or of about 7.0.

The compositions may have a pH of about 5.5, 6.0, 6.5, or about 7.0.

The compositions may have a pH of about 6.0.

The AlPO₄ adjuvant may have a PZC of about 4.5.

In some embodiments, AlPO₄ adjuvants of the disclosure are used with fHBP having isoelectric point (pI) ranging from about 5 to about 7.

In some embodiments, the difference between the PZC of the AlPO₄ adjuvant and the pI of the fHBP antigen may range from about 0.1 to about 2.8, or from about 0.5 to about 2.5, or from about 0.8 to about 2.1, or from about 0.96 to about 1.36.

In some embodiments, the difference between the PZC of the AlPO₄ adjuvant and the pI of the fHBP antigen may be of about 0.96 or of about 1.66.

The isoelectric point of an fHBP antigen may be determined empirically by a technique such as isoelectric focusing. More conveniently, however, the isoelectric point is a theoretical isoelectric point. This may be calculated using pKa values of amino acids described in Bjellqvist et al. ((1993) Electrophoresis 14:1023-31) and using the relevant ExPASy tool (Gasteiger et al. (2005) Protein Identification and Analysis Tools on the ExPASy Server in The Proteomics Protocols Handbook (ed. John M. Walker), Humana Press (2005)).

### Antigens

The immunogenic compositions disclosed herein comprise a combination of Neisseria meningitidis serogroup B antigens. The combination comprises at least one factor H binding protein (fHBP) A and at least one factor H binding protein (fHBP) B, and an aluminum hydroxyphosphate (AlPO₄) adjuvant, the AlPO₄ adjuvant having a point of zero charge (PZC) below 5.

fHBP

The meningococcal fHBP, also named lipoprotein 2086 (LP2086), ORF2086, and Genome-derived Neisserial antigen (GNA) 1870, or “741”, is a lipoprotein expressed on the surface of almost all invasive meningococcal isolates. fHBP is an important virulence factor, as it binds to the human complement factor H (fH), a negative regulator of the alternative complement pathway (Seib et al., Expert Rev Vaccines. 2015; 14(6):841-59). The binding of fHBP to human fH enables the pathogen to escape alternative complement-mediated killing by the host innate immune system and to survive in human serum and blood.

Three major genetic and immunological fHBP variants have been described: variant 1, corresponding to subfamily B, and variants 2 and 3, both classified in subfamily A (Seib et al., Expert Rev Vaccines. 2015; 14(6):841-59). Further to the nomenclature provided by Pfizer (fHBP A and B) and Novartis (variants 1, 2 and 3), fHBPs are identified in the PubMLST database with a unique ID number. Although there is significant antigenic variability between fHBP subfamilies A and B, protein sequences within each subfamily are highly conserved (>86% sequence identity) among different strains. Each unique fHBP found in N. meningitidis is also assigned a fHBP peptide ID according to neisseria.org or pubmlst.org/neisseria/fHBP/website. Because the length of variant 2 (v.2) fHBP protein (from strain 8047, fHBP ID 77) and variant 3 (v.3) fHBP (from strain M1239, fHBP ID 28) differ by −1 and +7 amino acid residues, respectively, from that of MC58 (fHBP ID 1, that is selected as the reference sequence for numbering), the numbering used to refer to residues for v.2 and v.3 fHBP proteins differ from numbering based on the actual amino acid sequences of these proteins. Thus, for example, reference to a leucine residue (L) at position 166 of the v.2 or v.3 fHBP sequence refers to the residue at position 165 of the v.2 protein and at position 173 in the v.3 protein. Members of variants 1, 2 and 3 are present in approximately 65%, 25%, and 10% of the MenB clinical isolates causing invasive diseases, respectively. The ten most prevalent fHBP variants represented in the MenB strain global population account for approximately 80% of the invasive disease-causing strains in the United States and Europe combined (Bambini et al., Vaccine. 2009; 27(21):2794-803; Chang, J Infect 2019; S0163-4453(19):30272-5; Lucidarme, Clin Vaccine Immunol 2010; 17(6):919-29; and Murphy et al., The Journal of infectious diseases. 2009; 200(3):379-89; Wang et al., Vaccine. 2011; 29(29-30):4739-44).

The fHBPs in the present disclosure may be wild-type (naturally occurring) polypeptides or may be non-naturally occurring (modified by amino acid substitutions, insertions, or deletions), provided that the polypeptide can elicit an immune response.

The fHBPs to be used according to the present disclosure may be lipidated or non-lipidated fHBPs. Lipidated proteins usually comprise a specific peptide sequence for lipidation at their N-terminus. This sequence may be cleaved during the maturation stage of the protein. Lipidation signal peptides are specific to each kind of protein and to the cells of the host producing the protein.

The fHBP polypeptide is expressed in N. meningitidis as a precursor protein having a lipoprotein signal motif at its N-terminus. During processing, the motif is cleaved to leave an N-terminal cysteine residue that is co-translationally modified with a lipid anchor that tethers the protein to the neisserial outer membrane (McNeil et al. (2013) MMBR 77(2):234-252). For lipidated fHBPs, lipids attached to cysteines will usually include palmitoyl residues, e.g., as tripalmitoyl-S-glyceryl-cysteine (Pam3Cys), dipalmitoyl-S-glyceryl cysteine (Pam2Cys), N-acetyl (dipalmitoyl-S-glyceryl cysteine), etc.

To avoid lipidation of a recombinant protein, various techniques known in the art can be used. As an example, it may be possible to delete the lipidation signal peptide or to replace the lipidation signal peptide with signal peptide not recognized by the cells in which the protein is produced. U.S. Pat. No. 10,300,122 B2 describes the use of this technique for fHBP.

It is also possible to either substituting the codon encoding the N-terminal cysteine with a codon coding for another amino acid or to remove the codon encoding the N-terminal cysteine. For example, U.S. Pat. No. 10,300,122 B2 describes insertion of an ATG (methionine) codon at the 5′ end of the 5′-terminal codon of the open reading frame encoding the mature fHBP protein. This results in a polypeptide lacking a lipidatable N-terminal cysteine residue. Additionally, U.S. Pat. No. 9,724,402 B2 and U.S. Pat. No. 11,077,180 B2 disclose the production of non-lipidated fHBP in which the N-terminal cysteine residue is substituted with an amino acid that is not a cysteine residue.

The fHBPs to be used according to the present disclosure may be naturally occurring or non-naturally occurring proteins. “Non-naturally occurring proteins” refer to “man-made proteins” and encompass fHBP with heterologous components that are not found in nature, by contrast to naturally occurring proteins. Non-naturally occurring proteins may be chimeric proteins or mutated proteins. “Chimeric proteins” in the context of the present disclosure is intended to refer to protein comprising two or more different components, each derived from a different fHBP (e.g., variant 1, 2, or 3). Mutations in a mutated protein may include amino acid substitution, insertion, or deletion. In one embodiment, a mutation is an amino acid substitution.

Non-naturally occurring fHBPs suitable for the immunogenic compositions as disclosed herein are still able to elicit an immune response against fHBPs. In one embodiment, the non-naturally occurring fHBPs to be used according to the present disclosure may be mutated fHBPs. Mutations, such as amino acids substitutions, may be introduced to reduce or suppress the binding of the fHBP antigen to the factor H (fH) of the coagulation normally present in the blood of an individual. The reduction of the binding of mutated fHBPs to the fH may increase the amounts of antigens available and accessible to the immune system. This may, in return, improve the efficacy and efficiency of the immune response towards those antigens. Advantageously, the mutated fHBP may elicit an anti-fHBP polyclonal antibodies directed to fHBP epitopes within the fH binding site, which resulted in greater protective complement deposition activity than the antibodies elicited by the wild-type (WT) fHBP antigens, which targeted fHBP epitopes outside of the fH binding site.

In some embodiments, the fHBPs may be mutated fHBPs comprising at least one mutation reducing or suppressing the binding of the fHBPs to the human factor H (fH).

The non-naturally occurring fHBPs considered for the immunogenic compositions as disclosed herein may present a reduced affinity towards fH compared to the corresponding naturally occurring fHBP or an improved thermal stability. Affinity towards fH protein and thermal stability may be measured as disclosed in WO 2016/014719 A1 (as in Examples 1 or 3 of this document).

For convenience and clarity, except otherwise specifically indicated, the native, or naturally occurring, amino acid sequence of fHBP B24 (or fHBP ID 1 or v.1 fHBP of the N. meningitidis strain MC58) of sequence SEQ ID NO:6 is selected as a reference sequence for all naturally occurring and non-naturally occurring fHBP amino acid sequences herein. Therefore, in referring to an amino acid residue position in a fHBP, the position number used herein corresponds to the amino acid residue number of SEQ ID NO:6 (fHBP B24). Consequently, position number 1 refers to the first amino acid residue shown in SEQ ID NO:6, which is a cysteine. This is still true even in case where further amino acids would be added at the N-terminus of SEQ ID NO:6, before this cysteine.

In one embodiment, a mutation (e.g., an amino acid substitution) in the fHBP A or B antigens used within the present disclosure may be as disclosed in WO 2011/126863 A1, WO 2015/017817 A1, or WO 2016/014719 A1.

Immunogenic compositions as disclosed herein may comprise non-naturally occurring fHBPs that differ in amino acid sequence from a wild-type N. meningitidis fHBP by from 1 to 10 amino acids (e.g., by from 1, 2, 3, 4, 5, 6, 7, 8, 9, or 10 amino acids), from 10 amino acids to 15 amino acids, from 15 amino acids to 20 amino acids, from 20 amino acids to 30 amino acids, from 30 amino acids to 40 amino acids, or from 40 amino acids to 50 amino acids.

In some embodiments, an fHBP antigen may comprise an amino acid sequence having at least about 80%, at least about 85%, at least about 90%, at least about 95%, at least about 98%, at least about 99%, or at least about 99.5%, amino acid sequence identity to a reference fHBP sequence.

Identity (e.g., percent homology) may be determined using various known sequence comparison tools, such as any homology comparison software computing a pairwise sequence alignment, including for example, the Blast software of the National Center of Biotechnology Information (NCBI), such as by using default parameters. The identity is a global identity, i.e., an identity over the entire amino acid or nucleic acid sequences and not over portions thereof. Pairwise global alignment was defined by Needleman et al., Journal of Molecular Biology, 1970, pages 443-53, volume 48). For example, when starting from a polypeptide sequence and comparing to other polypeptide sequences, the EMBOSS-6.0.1 Needleman-Wunsch algorithm (available from http://emboss.sourceforge.net/apps/cvs/emboss/apps/needle.html) may be used to find the optimum alignment of two sequences along their entire length—a “Global alignment”.

The fHBP antigens to be used in immunogenic compositions disclosed herein may be obtained as disclosed in WO 2016/014719 A1. The fHBPs may be obtained as recombinant proteins from recombinant expression vectors (or constructs) transfected into a host cell for production, for example an E. coli strain. Suitable vectors for transferring and expressing fHBP-encoding nucleic acid may vary in composition. Integrative vectors can be conditionally replicative plasmids, suicide plasmids, bacteriophages, and the like.

A construct may include various elements, including for example, promoters, selectable genetic markers (e.g., genes conferring resistance to antibiotics such as kanamycin, erythromycin, chloramphenicol, or gentamycin), origin of replication sites (to promote replication in a host cell, e.g., a bacterial host cell), and the like. The choice of vector will depend upon a variety of factors such as the type of cell in which propagation is desired and the purpose of propagation. Certain vectors are useful for amplifying and making large amounts of the desired DNA sequence. Other vectors are suitable for expression in cells in culture. The choice of appropriate vector is well within the skill of the art. Many such vectors are available commercially.

In one example, a vector may be an expression vector based on episomal plasmids comprising selectable drug resistance markers and elements that provide for autonomous replication in different host cells (e.g., in both E. coli and N. meningitidis). One example of such a “shuttle vector” is the plasmid pFPIO (Pagotto et al. (2000) Gene 244: 13-19). Vectors can provide for extrachromosomal maintenance in a host cell or can provide for integration into the host cell genome. Vectors are amply described in numerous publications well known to those in the art, including, e.g., Short Protocols in Molecular Biology, (1999) F. Ausubel, et al., eds., Wiley & Sons. Vectors may provide for expression of the nucleic acids encoding the subject fHBP, may provide for propagating the subject nucleic acids, or both.

Examples of vectors that may be used include but are not limited to those derived from recombinant bacteriophage DNA, plasmid DNA or cosmid DNA. For example, plasmid vectors such as pBR322, pUC 19/18, pUC 118, 119 and the M13 mp series of vectors may be used. pET21 is also an expression vector that may be used. Bacteriophage vectors may include λgtl0, λgtl 1, λgtl8-23, λZAP/R and the EMBL series of bacteriophage vectors. Further vectors that may be utilized include, but are not limited to, pJB8, pCV 103, pCV 107, pCV 108, pTM, pMCS, pNNL, pHSG274, COS202, COS203, pWE15, pWE16 and the charomid 9 series of vectors.

A recombinant expression vector may comprise a nucleotide sequence encoding an fHBP operably linked to a transcriptional control element, e.g., a promoter. A promoter may be constitutive or inducible. A promoter may be adapted for use in a prokaryotic host cell or in a eukaryotic host cell.

An expression vector provides transcriptional and translational regulatory sequences, and may provide for inducible or constitutive expression, where the coding region is operably linked under the transcriptional control of the transcriptional initiation region, and a transcriptional and translational termination region. These control regions may be native to an fHBP from which the subject fHBP is derived or may be derived from exogenous sources. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7, and the like).

Expression vectors generally have convenient restriction sites located near the promoter sequence to provide for the insertion of nucleic acid sequences encoding proteins of interest. Constructs (recombinant vectors) can be prepared by, for example, inserting a polynucleotide of interest into a construct backbone, typically by means of DNA ligase attachment to a cleaved restriction enzyme site in the vector. Alternatively, the desired nucleotide sequence can be inserted by homologous recombination or site-specific recombination. Typically, homologous recombination may be accomplished by attaching regions of homology to the vector on the flanks of the desired nucleotide sequence, while site-specific recombination can be accomplished through use of sequences that facilitate site-specific recombination (e.g., Cre-lox, att sites, etc.). Nucleic acids comprising such sequences can be added by, for example, ligation of oligonucleotides, or by polymerase chain reaction using primers comprising both the region of homology and a portion of the desired nucleotide sequence.

In addition, the expression construct may include additional elements. For example, the expression vector may have one or two replication systems, thus allowing it to be maintained in organisms, for example in mammalian or insect cells for expression and in a prokaryotic host for cloning and amplification. In addition, the expression construct may comprise a selectable marker gene to allow the selection of transformed host cells. Selection genes are well known in the art and will vary with the host cell used.

Amino acid substitutions may be introduced into fHBP sequences by any technique known in the art. For example, amino acid substitution may be obtained as disclosed in WO 2011/126863 A1, WO 2015/017817 A1, or WO 2016/014719 A1. In other exemplary embodiments, amino acids substitution may be obtained as disclosed in WO 2015/128480, WO 2010/046715, WO 2016/008960, WO 2020/030782, or WO 2011/051893.

Recombinant fHBPs may be obtained in purified form from culture by any purification methods known in the art, as for example described in the Examples section.

In one embodiment, an fHBP A and/or an fHBP B may be present in immunogenic compositions as disclosed herein in an amount from about 20 μg/dose to about 200 μg/dose, or from about 25 μg/dose to about 180 μg/dose, or from about 40 μg/dose to about 140 μg/dose, or from about 50 μg/dose to about 120 μg/dose, or from about 75 μg/dose to about 100 μg/dose. In one embodiment, an fHBP A and/or an fHBP B may be present in an amount at about 25 μg/dose, or at about 50 μg/dose, or at about 100 μg/dose.

fHBP B

An immunogenic composition as disclosed herein may comprise at least one fHBP B variant antigen. The at least one fHBP B may be a lipidated or a non-lipidated protein. An fHBP B may be a lipidated protein. An fHBP B may be a non-lipidated protein.

An fHBP B may be a naturally or a non-naturally occurring fHBP. An fHBP B may be a naturally occurring fHBP. In another embodiment, an fHBP B may be a non-naturally occurring fHBP.

An fHBP B may be a non-lipidated, non-naturally occurring fHBP.

An fHBP B protein may be a protein comprising at least about 85%, at least about 90%, at least about 95%, at least about 98%, at least about 99%, at least about 99.5% or about 100% amino acid sequence identity to SEQ ID NO: 3. A non-naturally occurring fHBP B01 protein is not 100% identical to fHBP B01 or SEQ ID NO. 3.

A non-naturally occurring fHBP B may be a chimeric protein as disclosed in WO 2011/126863 A1 or WO 2015/017817 A1 in or a mutated fHBP B protein as disclosed in WO 2016/014719 A1, WO 2011/051893, or in WO 2020/030782. In one exemplary embodiment, a fHBP B may be a mutated protein.

A non-naturally occurring fHBP B may be a mutated protein. A mutated fHBP B may be a non-lipidated protein.

A mutated fHBP B may differ in amino acid sequence from a wild-type N. meningitidis fHBP B, such as fHBP B01, by from 1 to 10 amino acids (e.g., by from 1, 2, 3, 4, 5, 6, 7, 8, 9, or 10 amino acids), from 10 amino acids to 15 amino acids, from 15 amino acids to 20 amino acids, from 20 amino acids to 30 amino acids, from 30 amino acids to 40 amino acids, or from 40 amino acids to 50 amino acids.

A mutated fHBP B may be a mutated protein comprising at least one mutation reducing or suppressing the binding of the fHBP B to the human factor H (fH).

A mutated fHBP B may comprise at least about 85% amino acid sequence identity to SEQ ID NO: 3.

A mutated fHBP B may be a mutated protein comprising at least about 85%, at least about 90%, at least about 95%, at least about 98%, or at least about 99%, or at least about 99.5% amino acid sequence identity to SEQ ID NO: 3. A mutated fHBP B protein is not 100% identical to fHBP B01 or SEQ ID NO:3.

A mutated fHBP B may comprise at least one amino acid substitution selected from at least one of: a) an amino acid substitution of the glutamine at amino acid 38 (Q38); b) an amino acid substitution of the glutamic acid at amino acid 92 (E92); c) an amino acid substitution of the arginine at amino acid 130 (R130); d) an amino acid substitution of the serine at amino acid 223 (S223); and e) an amino acid substitution of the histidine at amino acid 248 (H248), based on the numbering of SEQ ID NO:6.

A mutated fHBP B may comprise at least one amino acid deletion or substitution in any one of the following positions as disclosed in WO 2011/051893 with regard to the numbering of the fHBP sequence identified as SEQ ID NO: 4 in WO 2011/051893 (mature lipoprotein form of fHBP B24): D37, K45, T56, E83, E95, E112, K122, V124, R127, T139, F141, D142, K143, I198, S211, L213, K219, N43, D116, H119, S221 and K241.

A mutated fHBP B may comprise at least one amino substitution in any one of the following positions as disclosed in WO 2020/030782 with regard to the numbering of the fHBP sequence identified as SEQ ID NO: 2 in WO 2020/030782 (mature lipoprotein form of fHBP B09): E211, S216 or E232.

A mutated fHBP B may comprise at least one of the following amino substitutions in any one of the following positions as disclosed in WO 2020/030782 with regard to the numbering of the fHBP sequence identified as SEQ ID NO: 2 in WO 2020/030782 (mature lipoprotein form of fHBP B09): E211A, S216R or E232A.

A mutated fHBP B may comprise at least one amino substitution in any one of the following positions as disclosed in WO 2020/030782 with regard to the numbering of the fHBP sequence identified as SEQ ID NO: 6 in WO 2020/030782 (mature lipoprotein form of fHBP B44): E214, S219 or E235.

A mutated fHBP B may comprise at least one of the following amino substitutions in any one of the following positions as disclosed in WO 2020/030782 with regard to the numbering of the fHBP sequence identified as SEQ ID NO: 2 in WO 2020/030782 (mature lipoprotein form of fHBP B44): E214A, S219R or E235A.

A mutated fHBP B protein may comprise at least one of the following amino substitution in any one of the following positions as disclosed in WO2010046715 with regard to the numbering of the fHBP sequence identified as SEQ ID NO: 1 in WO2010046715 (mature lipoprotein form of fHBP 24: 103, 106, 107, 108, 109, 145, 147, 149, 150, 154, 156, 157, 180, 181, 182, 183, 184, 185, 191, 193, 194, 195, 196, 199, 262, 264, 266, 267, 268, 272, 274, 283, 285, 286, 288, 289, 302, 304 306, 311 and 313. In one embodiment, one or more amino acids which may be changed in the factor H binding protein may be selected from the group comprising amino acid number 103, 106, 107, 108, 180, 181, 183, 184, 185, 191, 193, 195, 262, 264, 266, 272, 274, 283, 286, 304 and 306 with regard to the numbering of the fHBP sequence identified as SEQ ID NO: 1 in WO2010046715.

An amino acid substitution of the glutamine at amino acid 38 (Q38) may be a Q38R substitution (R: arginine). Other amino acids with positively charged or aromatic side chains, such as lysine, histidine, phenylalanine, tyrosine or tryptophan, also may be substituted at this position. Thus, in some cases, the fHBP may comprise a Q38K substitution, a Q38H substitution, a Q38F substitution, a Q38Y substitution, or a Q38W substitution.

An amino acid substitution of the glutamic acid at amino acid 92 (E92) may be a E92K substitution. Other amino acids with positively charged or aromatic side chains, such as arginine, histidine, phenylalanine, tyrosine or tryptophan, also may be substituted at this position. Thus, for example, in some cases the fHBP variant may comprise an E92R substitution, an E92H substitution, an E92F substitution, an E92Y substitution, or an E92W substitution.

An amino acid substitution of the arginine at amino acid 130 (R130) may be a R130G substitution (G: glycine). Other amino acids with negatively charged or aromatic side chains, such as aspartate, glutamate, phenylalanine, tyrosine, or tryptophan, may also be substituted at R130. Thus, for example, in some cases the fHBP variant may comprise an R130D substitution, an R130E substitution, an R130F substitution, an R130Y substitution, or an R130W substitution.

An amino acid substitution of the serine at amino acid 223 (S223) may be an S223R substitution (R: arginine). Other amino acids with positively charged or aromatic side chains, such as lysine, histidine, phenylalanine, tyrosine or tryptophan, also may be substituted at this position. Thus, for example, in some cases, the fHbp variant comprises an S223K substitution, an S223H substitution, an S223F substitution, an S223Y substitution, or an S223W substitution.

In one exemplary embodiment, an amino acid substitution of the histidine at amino acid 248 (H248) may be an H248L substitution (L: leucine). Other amino acids with non-polar, negatively charged or aromatic side chains, such as isoleucine, valine, aspartate, glutamate, phenylalanine, tyrosine or tryptophan, also may be substituted at H248. Thus, for example, in some cases, the fHBP may comprise an H248I substitution, an H248V substitution, an H248D substitution, an H248E substitution, an H248F substitution, an H248Y substitution, or an H248W substitution.

In another embodiment, a mutated fHBP B may comprise at least the amino acid substitution H248L. A mutated fHBP B may comprise only the amino acid substitution H248L, based on the numbering of SEQ ID NO:6.

A mutated fHBP B may be a non-lipidated mutated fHBP B comprising at least one of the amino acid substitutions selected in the group consisting of a) an amino acid substitution of the glutamine at amino acid 38 (Q38); b) an amino acid substitution of the glutamic acid at amino acid 92 (E92); c) an amino acid substitution of the arginine at amino acid 130 (R130); d) an amino acid substitution of the serine at amino acid 223 (S223); and e) an amino acid substitution of the histidine at amino acid 248 (H248), based on the numbering of SEQ ID NO:6.

A mutated fHBP B may be a non-lipidated mutated fHBP B comprising at least one of the amino acid substitutions selected in the group consisting of a Q38R substitution, a Q38K substitution, a Q38H substitution, a Q38F substitution, a Q38Y substitution, a Q38W substitution, a E92K substitution, a E92R substitution, a E92H substitution, a E92F substitution, a E92Y substitution, a E92W substitution, a R130G substitution, a R130D substitution, a R130E substitution, a R130F substitution, a R130Y substitution, a R130W substitution, a S223R substitution, a S223K substitution, a S223H substitution, a S223F substitution, a S223Y substitution, a S223W substitution, a H248L substitution, a H248I substitution, a H248V substitution, a H248D substitution, a H248E substitution, a H248F substitution, a H248Y substitution, or a H248W substitution, based on the numbering of SEQ ID NO:6.

A mutated fHBP B may be a non-lipidated mutated fHBP B comprising at least the amino acid substitution H248L, based on the numbering of SEQ ID NO:6. In another exemplary embodiment, a non-lipidated mutated fHBP B protein may comprise only the amino acid substitution H248L, based on the numbering of SEQ ID NO:6.

A fHBP B may be a non-lipidated and mutated protein comprising at least about 85%, at least about 90%, at least about 95%, at least about 98%, or at least about 99% amino acid sequence identity to SEQ ID NO: 3 and comprising at least the amino acid substitution H248L, based on the numbering of SEQ ID NO:6. The non-lipidated mutated fHBP B may comprise only the amino acid substitution H248L, based on the numbering of SEQ ID NO:6.

A mutated, non-lipidated, fHBP B may comprise at least about 85%, at least about 90%, at least about 95%, at least about 98%, at least about 99%, or at least about 99.5% amino acid sequence identity to SEQ ID NO: 4.

In another embodiment, a mutated, non-lipidated, fHBP B may comprise or consist of SEQ ID NO: 4.

A mutated, non-lipidated, fHBP B may comprise at least about 85%, at least about 90%, at least about 95%, at least about 98%, at least about 99%, or at least about 99.5% amino acid sequence identity to SEQ ID NO: 9.

In another embodiment, a mutated, non-lipidated, fHBP B may comprise or consist of SEQ ID NO: 9.

The fHBP B may have an isoelectric point (pI) above the PZC of the AlPO₄ adjuvant.

The fHBP B may have an isoelectric point (pI) ranging from about 5.0 to about 7.0, or from 5.2 to about 6.5, or from about 5.3 to about 60., or is about 5.5, or else 5.46.

The fHBP B may have an isoelectric point (pI) of about 5.46.

In some embodiments, the difference between the PZC of the AlPO₄ adjuvant and the pI of the fHBP B may range from about 0.1 to about 2.9, or from about 0.4 to about 2.7, or from about 0.6 to about 2.2, or from about 0.7 to about 1.7, or from about 0.9 to about 1.2.

The difference between the PZC of the AlPO₄ adjuvant and the pI of the fHBP B may be of about 0.66 or of about 1.16.

The difference between the PZC of the AlPO₄ adjuvant and the pI of the fHBP B may be of about 0.66.

The difference between the PZC of the AlPO₄ adjuvant and the pI of the fHBP B may be of about 0.96.

The difference between the PZC of the AlPO₄ adjuvant and the pI of the fHBP B may be of about 1.16.

In one embodiment, a fHBP B may be present in immunogenic compositions as disclosed herein in an amount from about 20 μg/dose to about 200 μg/dose, or from about 25 μg/dose to about 180 μg/dose, or from about 40 μg/dose to about 140 μg/dose, or from about 50 μg/dose to about 120 μg/dose, or from about 75 μg/dose to about 100 μg/dose.

In one embodiment, a fHBP B may be present in an amount at about 25 μg/dose, or at about 50 μg/dose, or at about 100 μg/dose.

fHBP A

An immunogenic composition disclosed herein may comprise at least one fHBP A variant antigen. The at least one fHBP A may be a lipidated or a non-lipidated protein. A fHBP A may be a lipidated protein. A fHBP A may be a non-lipidated protein.

The fHBP A and the fHBP B may be both lipidated. In one embodiment, the fHBP A and the fHBP B may be both non-lipidated. Alternatively, the fHBP A may be lipidated and the fHBP B may be non-lipidated. Still alternatively, the fHBP A may be non-lipidated and the fHBP B may be lipidated.

An fHBP A may be a naturally or a non-naturally occurring fHBP. An fHBP A may be a naturally occurring fHBP. In another embodiment, an fHBP A may be a non-naturally occurring fHBP.

The fHBP A and the fHBP B may be both naturally occurring fHBP. The fHBP A and the fHBP B may be both non-naturally occurring fHBP. Alternatively, the fHBP A may be naturally occurring fHBP and the fHBP B may be non-naturally occurring fHBP. Still alternatively, the fHBP A may be non-naturally occurring fHBP and the fHBP B may be naturally occurring fHBP.

An fHBP A may be a non-lipidated, non-naturally occurring fHBP.

The fHBP A and the fHBP B may be both non-lipidated, non-naturally occurring fHBP.

An fHBP A may be a protein comprising at least about 85%, at least about 90%, at least about 95%, at least about 98%, at least about 99%, at least about 99.5% or about 100% amino acid sequence identity to SEQ ID NO: 1. A non-naturally occurring fHBP A05 is not 100% identical to fHBP A05 or SEQ ID NO: 1.

A non-naturally occurring fHBP A may be a chimeric protein as disclosed in WO 2011/126863 A1 or WO 2015/017817 A1 or a mutated fHBP A protein as disclosed in WO 2016/014719 A1, WO 2011/051893, WO 2016/008960 or WO 2015/128480. In one exemplary embodiment, a fHBP A may be a mutated protein.

A non-naturally occurring fHBP A may be a mutated protein. A mutated fHBP A may be a non-lipidated protein.

A mutated fHBP A may differ in amino acid sequence from a wild-type N. meningitidis fHBP A protein (e.g., fHBP A05) by from 1 to 10 amino acids (e.g., by from 1, 2, 3, 4, 5, 6, 7, 8, 9, or 10 amino acids), from 10 amino acids to 15 amino acids, from 15 amino acids to 20 amino acids, from 20 amino acids to 30 amino acids, from 30 amino acids to 40 amino acids, or from 40 amino acids to 50 amino acids.

A mutated fHBP A may comprise at least about 85% amino acid sequence identity to SEQ ID NO: 1.

A mutated fHBP A may be a mutated protein comprising at least one mutation reducing or suppressing the binding of the fHBP A to the human factor H (fH).

A mutated fHBP A may comprise at least about 85%, at least about 90%, at least about 95%, at least about 98%, or at least about 99%, or at least about 99.5% amino acid sequence identity to SEQ ID NO: 1. A mutated fHBP A05 protein is not 100% identical to fHBP A05 or SEQ ID NO: 1.

A mutated fHBP A may comprise at least one amino acid substitution selected from at least one of: a) an amino acid substitution of the asparagine at amino acid 115 (N115); b) an amino acid substitution of the aspartic acid at amino acid 121 (D121); c) an amino acid substitution of the serine at amino acid 128 (S128); d) an amino acid substitution of the phenylalanine at amino acid 129 (F129); e) an amino acid substitution of the leucine at amino acid 130 (L130); f) an amino acid substitution of the valine at position 131 (V131); g) an amino acid substitution of the glycine at position 133 (G133); h) an amino acid substitution of the lysine at position 219 (K219); and i) an amino acid substitution of the glycine at position 220 (G220), based on the numbering of SEQ ID NO:6.

A mutated fHBP A may comprise at least one amino acid deletion or substitution in any one of the following positions as disclosed in WO 2011/051893 with regard to the numbering of the fHBP sequence identified as SEQ ID NO: 5 in WO 2011/051893 (mature lipoprotein form of fHBP A19): D37, K45, T56, E83, E95, E112, S122, 1124, R127, T139, F141, N142, Q143, L197, D210, R212, K218, N43, N116, K119, T220 and/or 240. In one embodiment, the amino acid deletion or substitution is/are as disclosed in WO 2011/051893.

A mutated fHBP A may comprise at least one amino acid deletion or substitution in any one of the following positions as disclosed in WO 2016/008960 with regard to the numbering of the fHBP sequence identified as SEQ ID NO: 17 in WO 2016/008960 (A124 or variant 3.28 or ID28): S32, L126 and/or E243. One, two or three residues may be deleted. Alternatively, they may be substituted by a different amino acid. For example, Leu-126 can be substituted by any of the other 19 naturally-occurring amino acids. When a substitution is made, the replacement amino acid in some embodiments may be a simple amino acid such as glycine or alanine. In other embodiments, the replacement amino acid is a conservative substitution (e.g., it is made within the following four groups: (1) acidic i.e., aspartate, glutamate; (2) basic i.e., lysine, arginine, histidine; (3) non-polar i.e., alanine, valine, leucine, isoleucine, proline, phenylalanine, methionine, tryptophan; and (4) uncharged polar i.e., glycine, asparagine, glutamine, cysteine, serine, threonine, tyrosine). In other embodiments the substitution is non-conservative. In one embodiment, substitutions at the specified residues are as follows: S32V; L126R; and/or E243A.

A mutated fHBP A may comprise at least one amino acid deletion or substitution in any one of the following positions as disclosed in WO 2016/008960 with regard to the numbering of the fHBP sequence identified as SEQ ID NO: 5 in WO 2016/008960 (mature lipoprotein form): S32, L123 and/or E240. One, two, or three residues may be deleted. Alternatively, they may be substituted by a different amino acid. For example, Leu-123 can be substituted by any of the other 19 naturally-occurring amino acids. When a substitution is made, the replacement amino acid in some embodiments may be a simple amino acid such as glycine or alanine. In other instances, the replacement amino acid is a conservative substitution (e.g., it is made within the following four groups: (1) acidic i.e., aspartate, glutamate; (2) basic i.e., lysine, arginine, histidine; (3) non-polar i.e., alanine, valine, leucine, isoleucine, proline, phenylalanine, methionine, tryptophan; and (4) uncharged polar i.e., glycine, asparagine, glutamine, cysteine, serine, threonine, tyrosine). In other embodiments the substitution is non-conservative. In one embodiment, the substitutions at the specified residues may be as follows: S32V; L123R; and/or E240A.

A mutated fHBP A may comprise at least one amino acid deletion or substitution in any one of the following positions as disclosed in WO 2015/128480 with regard to the numbering of the fHBP sequence identified as SEQ ID NO: 5 in WO 2015/128480 (fHBP A19 or v2.16 or ID16): S32, V33, L39, L41, F69, V100, 1113, F122, L123, V124, S125, G126, L127, G128, S151, H239, and/or E240. In one embodiment, residues for mutation may be S32, V100, L123, V124, S125, G126, L127, G128, H239, and/or E240. Mutations at these residues give proteins having good stability compared to wild-type fHBP A. In one embodiment, residues for mutations may be S32, L123, V124, S125, G126, L127, and/or G128. In one embodiment, residues for mutations may be S32, L123, V124, S125, G126, L127, and/or G128. In another embodiment, residues S32 and/or L123 may be mutated, e.g., S32V and/or L123. Where one or more of V100, S125, and/or G126 is mutated, mutation of a residue outside this trio may be also introduced.

The specified residue can be deleted, but preferably it is substituted by a different amino acid. For example, Ser-32 can be substituted by any of the other 19 naturally-occurring amino acids. When a substitution is made, the replacement amino acid in some embodiments may be a simple amino acid such as glycine or alanine. In other embodiments, the replacement amino acid is a conservative substitution (e.g., it is made within the following four groups: (1) acidic i.e., aspartate, glutamate; (2) basic i.e., lysine, arginine, histidine; (3) non-polar i.e., alanine, valine, leucine, isoleucine, proline, phenylalanine, methionine, tryptophan; and (4) uncharged polar i.e., glycine, asparagine, glutamine, cysteine, serine, threonine, tyrosine). In other embodiments the substitution is non-conservative. In some embodiments the substitution does not use alanine.

Substitutions at the specified residues may be as follows: S32V; V33C; L39C; L41C; F69C; V100T; I113S; F122C; L123R; V124I; S125G or S125T; G126D; L127I; G128A; S151C; H239R; or E240H.

A mutated fHBP A may comprise at least one amino acid deletion or substitution in any one of the following positions, as disclosed in WO 2015/128480 with regard to the numbering of the fHBP sequence identified as SEQ ID NO: 17 in WO 2015/128480 (A124 or variant 3.28 or ID28): S32, V33, L39, L41, F72, V103, T116, F125, L126, V127, S128, G129, L130, G131, S154, H242, and/or E243. In one embodiment, residues for mutation may be S32, V103, L126, V127, S128, G129, L130, G131, H242, and/or E243 In one embodiment, residues for mutations may be S32, L126, V127, S128, G129, L130, and/or G131. In another embodiment, residues S32, L126, V127, S128, G129, L130, and/or G131 may be mutated, as for example residues S32 and/or L126 e.g., S32V and/or L126R.

The specified residue can be deleted, but preferably it is substituted by a different amino acid. For example, Ser-32 can be substituted by any of the other 19 naturally-occurring amino acids. When a substitution is made, the replacement amino acid in some embodiments may be a simple amino acid such as glycine or alanine. In other embodiments, the replacement amino acid is a conservative substitution (e.g., it is made within the following four groups: (1) acidic i.e., aspartate, glutamate; (2) basic i.e., lysine, arginine, histidine; (3) non-polar i.e., alanine, valine, leucine, isoleucine, proline, phenylalanine, methionine, tryptophan; and (4) uncharged polar i.e., glycine, asparagine, glutamine, cysteine, serine, threonine, tyrosine). In other embodiments the substitution is non-conservative. In some embodiments the substitution does not use alanine.

Substitutions at the specified residues may be as follows: S32V; I33C; L39C; L41C; F72C; V103T; T116S; F125C; L126R; V127I; S128G or S128T; G129D; L130I; G131A; S154C; H242R; E243H.

An amino acid substitution of the asparagine at amino acid 115 (N115) may be a N115I substitution (I: isoleucine). Other amino acids with non-polar, positively charged or aromatic side chains, such as valine, leucine, lysine, arginine, histidine, phenylalanine, tyrosine or tryptophan, also may be substituted at this position. Thus, in some cases, the fHBP may comprise an N115V substitution, an N115L substitution, an N115K substitution, an N115R substitution, an N115H substitution, an N115F substitution, an N115Y substitution, or an N115W substitution.

An amino acid substitution of the aspartic acid at amino acid 121 (D121) may be a D121G substitution (G: glycine). Other amino acids with non-polar, positively charged or aromatic side chains, such as leucine, isoleucine, valine, lysine, arginine, histidine, phenylalanine, tyrosine or tryptophan, also may be substituted at this position. Thus, for example, in some cases the fHBP variant may comprise a D121L substitution, a D121I substitution, a D121V substitution, a D121K substitution, a D121R substitution, a D121H substitution, a D121F substitution, a D121Y substitution, or a D121W substitution.

An amino acid substitution of the serine at amino acid 128 (S128) may be a S128T substitution (T: threonine). Other amino acids with polar, charged or aromatic side chains, such as methionine, asparagine, glutamine, aspartate, glutamate, lysine, arginine, histidine, phenylalanine, tyrosine or tryptophan, also may be substituted at this position. Thus, for example, in some cases the fHBP variant may comprise an S128M substitution, an S128N substitution, an S128D substitution, an S128E substitution, an S128K substitution, an S128R substitution, an S128H substitution, an S128F substitution, an S128Y substitution, or an S128W substitution.

A mutated fHBP A may comprise an amino acid substitution of the leucine at amino acid 130 (L130). An amino acid substitution of the leucine at amino acid 130 (L130) may be a L130R substitution (R: arginine).

A mutated fHBP A may comprise an amino acid substitution of the valine at amino acid 131 (V131). Other amino acids with charged or aromatic side chains, such as glutamate, lysine, arginine, histidine, phenylalanine, tyrosine or tryptophan, also may be substituted at this position. Thus, for example, in some cases, the fHBP may comprise a V131E substitution, a V131K substitution, a V131R substitution, a V131H substitution, a V131F substitution, a V131Y substitution, or a V131W substitution.

A mutated fHBP A may comprise an amino acid substitution of the glycine at amino acid 133 (G133). An amino acid substitution of the glycine at amino acid 133 (G133) may be a G133D substitution (D: aspartic acid).

A mutated fHBP A may comprise an amino acid substitution of the lysine at position 219 (K219). Other amino acids with polar, negatively charged or aromatic side chains, such as glutamine, aspartate, glutamate, phenylalanine, tyrosine or tryptophan, also may be substituted at this position. Thus, for example, in some cases, the fHBP may comprise a K219Q substitution, a K219D substitution, a K219E substitution, a K219F substitution, a K219Y substitution, or a K219W substitution.

An amino acid substitution of the glycine acid at amino acid 220 (G220) may be a G220S substitution (S: serine). Other amino acids with polar, charged or aromatic side chains, such as asparagine, glutamine, aspartate, glutamate, lysine, arginine, histidine, phenylalanine, tyrosine or tryptophan, also may be substituted at this position. Thus, for example, in some cases, the mutated fHBP A may comprise a G220N substitution, a G220Q substitution, a G220D substitution, a G220E substitution, a G220K substitution, a G220R substitution, a G220H substitution, a G220F substitution, a G220Y substitution, or a G220W substitution.

In one exemplary embodiment, an amino acid substitution of the leucine at amino acid 130 (L130) may be a L130R substitution (R: arginine).

In one exemplary embodiment, an amino acid substitution of the glycine at amino acid 133 (G133) may be a G133D substitution (D: aspartic acid).

In one exemplary embodiment, an amino acid substitution of the glycine at position 220 (G220) may be a G220S substitution (S: serine).

In one exemplary embodiment, an amino acid substitution of the phenylalanine at position 129 (F129) may be a F129S substitution (S: serine).

A mutated fHBP A may comprise at least one of the amino acid substitutions selected in the group consisting of G220S, L130R, and G133D, based on the numbering of SEQ ID NO:6. In another embodiment, a mutated fHBP A may comprise at least the three amino acid substitutions selected in the group consisting of G220S, L130R, and G133D, based on the numbering of SEQ ID NO:6. In another embodiment, a mutated fHBP A protein may comprise only the three amino acid substitutions G220S, L130R, and G133D, based on the numbering of SEQ ID NO:6.

A mutated fHBP A may be a non-lipidated mutated fHBP A comprising at least one of the amino acid substitutions selected in the group consisting of a) an amino acid substitution of the asparagine at amino acid 115 (N115); b) an amino acid substitution of the aspartic acid at amino acid 121 (D121); c) an amino acid substitution of the serine at amino acid 128 (S128); d) an amino acid substitution of the leucine at amino acid 130 (L130); e) an amino acid substitution of the valine at position 131 (V131); f) an amino acid substitution of the glycine at position 133 (G133); g) an amino acid substitution of the lysine at position 219 (K219); and h) an amino acid substitution of the glycine at position 220 (G220), based on the numbering of SEQ ID NO:6.

A mutated fHBP A may be a non-lipidated mutated fHBP A comprising at least one of the amino acid substitutions selected in the group consisting of a N115I substitution, a N115V substitution, a N115L substitution, a N115K substitution, a N115R substitution, an N115H substitution, an N115F substitution, an N115Y substitution, an N115W substitution, a D121G substitution, a D121L substitution, a D121I substitution, a D121V substitution, a D121K substitution, a D121R substitution, a D121H substitution, a D121F substitution, a D121Y substitution, a D121W substitution, a S128T substitution, a S128M substitution, a S128N substitution, a S128D substitution, a S128E substitution, a S128K substitution, a S128R substitution, a S128H substitution, a S128F substitution, a S128Y substitution, a S128W substitution, a L130R substitution, a V131E substitution, a V131K substitution, a V131R substitution, a V131H substitution, a V131F substitution, a V131Y substitution, a V131W substitution, a G133D substitution, a K219Q substitution, a K219D substitution, a K219E substitution, a K219F substitution, a K219Y substitution, a K219W substitution, a G220S substitution, a G220N substitution, a G220Q substitution, a G220D substitution, a G220E substitution, a G220K substitution, a G220R substitution, a G220H substitution, a G220F substitution, a G220Y substitution, or a G220W substitution, based on the numbering of SEQ ID NO:6.

A mutated fHBP A may be a non-lipidated mutated fHBP A comprising at least one of the amino acid substitutions selected in the group consisting of G220S, L130R, and G133D, based on the numbering of SEQ ID NO:6. In another embodiment, a mutated, non-lipidated fHBP A may comprise at least the three amino acid substitutions selected in the group consisting of G220S, L130R, and G133D, based on the numbering of SEQ ID NO:6. In another exemplary embodiment, a non-lipidated mutated fHBP A may comprise only the three amino acid substitutions G220S, L130R, and G133D, based on the numbering of SEQ ID NO:6.

In one exemplary embodiment, a fHBP A may be a non-lipidated and mutated protein comprising at least about 85%, at least about 90%, at least about 95%, at least about 98%, at least about 99%, or at least about 99.5% amino acid sequence identity to SEQ ID NO: 1 and comprising at least one of the amino acid substitutions selected in the group consisting of G220S, L130R, and G133D, based on the numbering of SEQ ID NO:6. The mutated, non-lipidated fHBP A protein may comprise at least the three amino acid substitutions selected in the group consisting of G220S, L130R, and G133D, based on the numbering of SEQ ID NO:6. In another exemplary embodiment, the non-lipidated mutated fHBP A protein may comprise only the three amino acid substitutions G220S, L130R, and G133D, based on the numbering of SEQ ID NO:6.

In one embodiment a mutated, non-lipidated, fHBP A protein may comprise at least about 85%, at least about 90%, at least about 95%, at least about 98%, at least about 99%, or at least about 99.5% amino acid sequence identity to SEQ ID NO: 2.

In another embodiment, a mutated, non-lipidated, fHBP A protein may comprise or consist of SEQ ID NO: 2.

In one embodiment a mutated, non-lipidated, fHBP A protein may comprise at least about 85%, at least about 90%, at least about 95%, at least about 98%, at least about 99%, or at least about 99.5% amino acid sequence identity to SEQ ID NO: 8.

In another embodiment, a mutated, non-lipidated, fHBP A protein may comprise or consist of SEQ ID NO: 8.

The fHBP A may have an isoelectric point (pI) ranging from about 5 to about 7, or from 5.2 to about 6.5, or from about 5.4 to about 6, or is about 5.9, or else 5.86.

The fHBP A may have an isoelectric point (pI) of about 5.86.

In some embodiments, the difference between the PZC of the AlPO₄ adjuvant and the pI of the fHBP A may range from about 0.1 to about 2.9, or from about 0.4 to about 2.7, or from about 0.6 to about 2.2, or from about 0.7 to about 1.8, or from about 0.9 to about 1.6.

The difference between the PZC of the AlPO₄ adjuvant and the pI of the fHBP A may be of about 1.06 or of about 1.56.

The difference between the PZC of the AlPO₄ adjuvant and the pI of the fHBP A may be of about 1.06.

The difference between the PZC of the AlPO₄ adjuvant and the pI of the fHBP A may be of about 1.36.

The difference between the PZC of the AlPO₄ adjuvant and the pI of the fHBP A may be of about 1.56.

An fHBP A antigen may be present in immunogenic compositions as disclosed herein in an amount from about 20 μg/dose to about 200 μg/dose, or from about 25 μg/dose to about 180 μg/dose, or from about 40 μg/dose to about 140 μg/dose, or from about 50 μg/dose to about 120 μg/dose, or from about 75 μg/dose to about 100 μg/dose.

An fHBP A protein may be present in an amount at about 25 μg/dose, or at about 50 μg/dose, or at about 100 μg/dose.

### NadA

An immunogenic composition of the disclosure may further comprise at least one Neisseria adhesin A (NadA) protein.

Neisseria adhesin A (NadA, previously known as GNA1994) is a surface-exposed, trimeric protein forming oligomers that are anchored via a C-terminal transmembrane domain into the outer membrane. NadA is expressed with a signal peptide and has three main domains: (1) a COOH-terminal anchoring domain (β structure), which is also necessary for auto-translocation to the bacterial surface; (2) a probably coiled domain with a leucine zipper, which might mediate dimerization and oligomerization; (3) a NH(2)-terminal globular head domain. NadA plays a key role in extracellular adhesion and invasion of epithelial cells (Capecchi et al., Mol. Microbiol. 2005; 55:(687-98)). The sequences of NadA proteins from many N. meningitidis strains have been published, and the protein's activity as a Neisserial adhesin has been well documented. The NadA gene is present in approximately 50% of meningococcal isolates. NadA exhibits growth phase-dependent expression, with maximal expression levels occurring in the stationary phase.

The NadA protein was included in the published genome sequence for meningococcal serogroup B strain MC58 as gene NMB1994 (GenBank accession number GI:7227256).

A NadA polypeptide for use according to the present disclosure may be the wild-type polypeptide or may be modified by amino acid substitutions, insertions, or deletions, provided that the polypeptide can elicit an immune response against NadA.

A NadA protein to be used may be a N-terminally and/or C-terminally truncated NadA or NadA proteins comprising amino acids deletions or insertions, for example as disclosed in references WO 01/64920; WO 01/64922; or WO 03/020756.

A recombinant NadA protein to be used in an immunogenic composition as disclosed herein may be a NadA1 variant. As shown in the Examples, NadA1 was shown to induce a strong hSBA response.

NadA1 may be obtained from the NadA sequence of MenB MC58 strain.

A NadA protein may comprise or consist of the sequence SEQ ID NO: 7.

A NadA protein may comprise at least 190 consecutive amino acids from SEQ ID NO: 7, for example 200 or more, 210 or more, 220 or more, 230 or more, 240 or more, 250 or more consecutive amino acids from SEQ ID NO: 7, for example 260 or more, or 270 or more, or 280 or more, or 290 or more, or 300 or more, or 310 or more, or 320 or more, or 330 or more, or 340 or more, or 350 or more, or 360 or more amino acids from SEQ ID NO: 7.

A NadA protein may lack from 5 to 10 amino acids, or from 10 to 15, or from 15 to 20, or 25, or 30 or 35, or 40 or 45, or 50 or 55 amino acids from the C-terminus and/or the N-terminus, for example of SEQ ID NO: 7. Where N-terminus residues are deleted, such deletion should not remove the ability of NadA to adhere to human epithelial cells.

A NadA protein may lack the signal peptide at the N-terminus. For example, a NadA protein may lack 23 amino acids that the N-terminus, for example of SEQ ID NO: 7.

A NadA protein may lack the membrane anchoring peptide at its C-terminus. For example, a NadA protein may lack 55 amino acids at the C-terminus, for example of SEQ ID NO: 7.

NadA may be used in a monomeric or an oligomeric form, for example in its trimeric form.

For example, a NadA protein may be without its C-terminal membrane anchor (e.g., deletion of residues 308-362 for strain MC58 (SEQ ID NO: 7)). Expression of NadA without its membrane anchor domain in E. coli may results in secretion of the protein into the culture supernatant with concomitant removal of the 23 amino acids signal peptide (e.g., deletion of residues 2 to 24 of SEQ ID NO: 7, to leave a 284 amino acids protein—SEQ ID NO: 5).

A NadA protein may comprise at least about 85%, at least about 90%, at least about 95%, at least about 98%, at least about 99%, or at least about 99.5% amino acid sequence identity to SEQ ID NO: 5. In another embodiment, a NadA protein may comprise or consist of SEQ ID NO:5.

NadA proteins to be used in immunogenic compositions disclosed herein may be obtained according to any recombinant techniques known in the art, for example as previously disclosed. The NadA proteins may be obtained as recombinant proteins from recombinant expression vectors (or constructs) transfected into a host cell for production, for example an E. coli strain. Recombinant NadA may be obtained in purified form from culture by any purification methods known in the art, for example as described in the Examples section.

In one embodiment, a NadA protein may be present in an amount ranging from about 20 μg/dose to about 200 μg/dose, or from about 25 μg/dose to about 180 μg/dose, or from about 40 μg/dose to about 140 μg/dose, or from about 50 μg/dose to about 120 μg/dose, or from about 75 μg/dose to about 100 μg/dose. In one embodiment, a NadA protein may be present in an or at about 50 μg/dose.

dOMV

An immunogenic composition of the disclosure may further comprise at least one detergent-extracted Outer Membrane Vesicle (dOMV).

Immunogenic compositions as disclosed herein comprise a detergent-extracted Outer Membrane Vesicle (dOMV), also referred to as an Outer Membrane Protein Complex (OMPC). Usually, detergent-extracted Outer Membrane Vesicles are referred to as dOMV or OMV when used as antigens. They are referred to as OMPC when used as protein carrier.

OMPC was used as carrier protein platform for the polyribosylribitol phosphate (PRP) conjugate vaccine PedvaxHIB (Haemophilus influenzae type B vaccine) (Einhorn et al., Lancet (London, England). 1986; 2(8502):299-302; Moro et al., The Journal of pediatrics. 2015; 166(4):992-7) and for VAXELIS (diphtheria, tetanus, pertussis, poliomyelitis, hepatitis B and H. influenzae type B vaccine) (Syed, Paediatric drugs. 2017; 19(1):69-80).

dOMV are large proteolipid vesicles comprising integral outer member proteins and residual lipi-oligosaccharides (LOS) found in the bacterial outer membrane (Helting, Acta Pathol Microbiol Scand C, 1981; 89(2):69-78). More than 300 proteins may be identified in the dOMV. 75% of the total protein content of the dOMV are represented by 10 most abundant proteins, including the outer membrane proteins porin A (PorA) and porin B (PorB) which represent up to 50% of the total proteins.

The Neisseria meningitidis porin protein (Por) is an antigenic determinant for serovar typing. Two classes of Porins, PorA and PorB, and antigenically distinct variants within each class resulting from sequence variations in the por gene variable regions (VRs) encoding surface-exposed loops are identified.

dOMV suitable for the immunogenic compositions disclosed herein may be obtained from various MenB strains. dOMV may be isolated from a detergent-extract of a MenB strain. A suitable MenB strain may be a wild-type MenB strain or a MenB strain engineered to overexpress a Porin protein, such as a PorA or a PorB protein, and for example a PorA protein.

dOMV may be obtained from a MenB strain expressing PorA proteins.

dOMV may be obtained from MenB strains expressing a PorA VR2 subtype.

A PorA VR2 subtype may be a PorA VR2 type P1.2, P1.4, P1.7, P1.10, or P1.13 protein.

dOMV may be obtained from MenB strains expressing a PorA VR2 type P1.2 protein.

dOMV may be obtained from MenB strain expressing a PorA VR2 subtype and PorB P2.2a. A dOMV may be obtained from MenB strain expressing a PorA VR2 P1.2 and PorB P2.2a.

dOMV may comprise a PorA VR2 subtype and PorB P2.2a. A dOMV may comprise a PorA VR2 P1.2 and PorB P2.2a.

dOMV may comprise PorA VR2 P1.2 and PorB P2.2a and immunotype LOS L3,7. PorA and PorB may represent about 50% of the proteins of the dOMV.

dOMV may be obtained from a single MenB strain, or from different MenB strains. In the latter case, the MenB strains may express the same sub-type of PorA protein, or different PorA protein sub-type, or different type of pore protein, such as PorA and PorB proteins.

Useable MenB strains from which dOMV presenting the sought Porin protein may be identified for instance from the PubMLST database (https://pubmlst.org/). For example, a suitable MenB strain may be obtained by selecting within such database MenB strains from epidemic outbreaks, and then selecting within such subset the MenB strains having a gene coding for the porin protein of interest, such as PorA VR2 P1.2 protein. Then, it may be evaluated with known techniques in the art whether the selected strain(s) effectively expresses the porin protein of interest.

As examples of MenB strains suitable to obtain dOMV according to the disclosure, one may mention the following ones: NG H36, BZ 232, DK 353, B6116/77, BZ 163, 0085/00, NG P20, 0046/02, M11 40123, M12 240069, N5/99, 99M, or M07 240677.

In one exemplary embodiment, a MenB strain may be the MenB strain 99M expressing the PorA VR2 P1.2 protein subtype.

In one embodiment, a dOMV may comprise porin A (PorA) VR2 subtype P1.2.

In another embodiment, a dOMV may comprise outer membrane protein porin A (PorA) and/or outer membrane protein porin B (PorB). dOMV may comprise outer membrane protein porin A (PorA) and outer membrane protein porin B (PorB).

PorA may be present in an amount ranging from about 3% to about 15%, or in an amount of about 5% to about 9 or 10% relative to the total proteins present in said dOMV. PorB may be present in an amount ranging from about 30% to about 70%, or from about 35% to about 65%, or from about 38% to about 58% relative to the total proteins present in the dOMV.

In one embodiment, a dOMV may be obtained with a detergent-extraction method using at least a step of deoxycholate treatment.

A suitable method to obtain dOMV may be as disclosed in Helting et al. (Acta Pathol Microbiol Scand C. 1981 April; 89(2):69-78) or in Example 2 of U.S. Pat. No. 4,695,624. For example, bacteria culture may be centrifuged to obtain a pellet which may be then extracted with a detergent, for example a deoxycholate or sodium dodecyl sulfate (SDS) under heating, for example from about 50° C. to about 60° C., or at about 56° C., and for a time ranging from about 10 to about 20 minutes, or at about 15 minutes. Resulting materials may then be centrifugated, and the pellets may be further suspended and purified according to any methods known in the art.

dOMV may be present in an amount ranging from about 5 μg/dose to about 400 μg/dose, or from about 10 μg/dose to about 300 μg/dose, or from about 25 μg/dose to about 250 μg/dose, or from about 35 μg/dose to about 225 μg/dose, or from about 50 μg/dose to about 200 μg/dose, or from about 75 μg/dose to about 180 μg/dose, or from about 100 μg/dose to about 150 μg/dose, or from about 110 μg/dose to about 125 μg/dose.

In one embodiment, a dOMV may be present in an amount at about 25 μg/dose, or at about 50 μg/dose, or at about 125 μg/dose.

### Further Antigens

An immunogenic composition of the disclosure may comprise at least one further antigen.

A further antigen may be a saccharide antigen from N. meningitidis serogroup A, C, W135, Y and/or X conjugated to a carrier protein. In one embodiment, a composition of the disclosure may further comprise a combination of conjugates of MenA, MenC, MenW-135 and MenY capsular polysaccharides to a carrier protein.

In one embodiment, the further antigen may be a combination of conjugates of MenA, MenC, MenW-135 and MenY capsular polysaccharides to a carrier protein.

The carrier protein for the different capsular polysaccharide may be different or identical. Carrier proteins may include inactivated bacterial toxins such as diphtheria toxoid, CRM197, tetanus toxoid, pertussis toxoid, E. coli LT, E. coli ST, and exotoxin A from Pseudomonas aeruginosa. Bacterial outer membrane proteins such as, porins, transferrin binding proteins, pneumolysis, pneumococcal surface protein A (PspA), or pneumococcal adhesin protein (PsaA), could also be used. Other proteins, such as ovalbumin, keyhole limpit hemocyanin (KLH), bovine serum albumin (BSA), or purified protein derivative of tuberculin (PPD) may also be used as carrier proteins. It may be a CRM197 protein, a tetanus or a diphtheria toxoid. In one embodiment, it is a tetanus toxoid.

The conjugates may be a population comprising molecules with a molecular weight in the range of 700 kDa to 1400 kDa or 800 kDa to 1300 kDa.

Within each dose, the amount of an individual saccharide antigen may be between 1-50 μg measured as mass of saccharide. For example, a total of 40 μg of saccharides per dose may be administered. For example, 10 μg of each polysaccharide and approximately 55 μg of carrier protein, such as tetanus toxoid protein, may be administered.

The further antigen may be a combination of MenA, MenC, MenW-135 and MenY capsular polysaccharides each conjugated to a tetanus toxoid carrier protein, wherein the MenA polysaccharide is conjugated to the tetanus toxoid carrier via an adipic acid dihydrazide (ADH) linker while the MenC, MenW-135 and MenY polysaccharides are each directly conjugated to the tetanus toxoid carrier (TT).

In one embodiment, the further antigens may be a combination of conjugates of MenA, MenC, MenW-135 and MenY capsular polysaccharides to tetanus toxoid carrier protein. In an exemplary embodiment, the conjugated saccharide antigens from N. meningitidis serogroup A, C, W135 and/or Y may be as disclosed in WO 2018/045286 A1 or WO 2002/058737 A2.

In one embodiment, the further antigens are the ones of the commercially available MenACYW-TT conjugate vaccine MENQUADFI®.

### Immunogenic Compositions

An immunogenic composition discloses here may comprise a combination of meningococcal antigens, said combination comprising at least one factor H binding protein (fHBP) A protein, at least one fHBP B protein, at least one Neisseria adhesin A (NadA) protein, and at least one detergent-extracted Outer Membrane Vesicle (dOMV).

An immunogenic composition discloses here may comprise a combination of Neisseria meningitidis serogroup B antigens, said combination comprising at least one factor H binding protein (fHBP) A, at least one fHBP B, at least one Neisseria adhesin A (NadA) protein, and at least one detergent-extracted Outer Membrane Vesicle (dOMV). The fHBP A and/or the fHBP B may be non-lipidated.

The fHBP A may be a mutated protein comprising at least about 85% identity with SEQ ID NO: 1 and/or the fHBP B may be a mutated protein comprising at least about 85% identity with SEQ ID NO: 3.

The fHBP A may be a mutated protein comprising at least one mutation reducing or suppressing the binding of the fHBP A to the human factor H (fH) and/or the fHBP B may be a mutated protein comprising at least one mutation reducing or suppressing the binding of the fHBP B to the human factor H (fH).

The fHBP A may comprise at least one amino acid substitution selected from at least one of: a) an amino acid substitution of the asparagine at amino acid 115 (N115); b) an amino acid substitution of the aspartic acid at amino acid 121 (D121); c) an amino acid substitution of the serine at amino acid 128 (S128); d) an amino acid substitution of the phenylalanine at amino acid 129; e) an amino acid substitution of the leucine at amino acid 130 (L130); f) an amino acid substitution of the valine at position 131 (V131); g) an amino acid substitution of the glycine at position 133 (G133); h) an amino acid substitution of the lysine at position 219 (K219); and i) an amino acid substitution of the glycine at position 220 (G220), based on the numbering of SEQ ID NO:6, or comprises or consists of SEQ ID NO: 2 or comprises or consists of SEQ ID NO: 8, and/or the fHBP B protein may comprise at least one amino acid substitution selected from at least one of: a) an amino acid substitution of the glutamine at amino acid 38 (Q38); b) an amino acid substitution of the glutamic acid at amino acid 92 (E92); c) an amino acid substitution of the arginine at amino acid 130 (R130); d) an amino acid substitution of the serine at amino acid 223 (S223); and e) an amino acid substitution of the histidine at amino acid 248 (H248), based on the numbering of SEQ ID NO:6, or comprises or consists of SEQ ID NO: 4, or comprises or consists of SEQ ID NO: 9.

The fHBP A and/or the fHBP B may be present in an amount ranging from about 20 μg/dose to about 200 μg/dose, or from about 25 μg/dose to about 180 μg/dose, or from about 40 μg/dose to about 140 μg/dose, or from about 50 μg/dose to about 120 μg/dose, or from about 75 μg/dose to about 100 μg/dose, or at about 25 μg/dose, or at about 50 μg/dose, or at about 100 μg/dose.

The NadA protein may be NadA1 protein or may comprise at least about 85% identity with SEQ ID NO: 5 or comprises or consists of SEQ ID NO:5.

The NadA protein may be present in an amount ranging from about 20 μg/dose to about 200 μg/dose, or from about 25 μg/dose to about 180 μg/dose, or from about 40 μg/dose to about 140 μg/dose, or from about 50 μg/dose to about 120 μg/dose, or from about 75 μg/dose to about 100 μg/dose, or at about 50 μg/dose.

The dOMV may comprise porin A (PorA).

The dOMV may be present in an amount ranging from about 5 μg/dose to about 400 μg/dose, or from about 10 μg/dose to about 300 μg/dose, or from about 25 μg/dose to about 250 μg/dose, or from about 35 μg/dose to about 225 μg/dose, or from about 50 μg/dose to about 200 μg/dose, or from about 75 μg/dose to about 180 μg/dose, or from about 100 μg/dose to about 150 μg/dose, or from about 110 μg/dose to about 125 μg/dose, or at about 25 μg/dose, or at about 50 μg/dose, or at about 125 μg/dose.

The composition may comprise an adjuvant, for example an aluminum-based adjuvant, for example an aluminum-based adjuvant selected in a group comprising aluminum hydroxide adjuvant, aluminum phosphate adjuvant, sulphate aluminum salt adjuvant, aluminium hydroxyphosphate sulfate adjuvant, potassium aluminium sulfate adjuvant, aluminum hydroxycarbonate, a combination of aluminum hydroxide and magnesium hydroxide, and mixtures thereof, for example being an aluminum phosphate adjuvant.

The compositions may have a pH from 0.6 to 2.9 units from the PZC of the AlPO₄ adjuvant, or from 1.2 to 2.9 units from the PZC of the adjuvant. The compositions may have a pH from 0.6 to 2.9 units from the PZC of the AlPO₄ adjuvant, or from 1.0 to 2.8, or from 1.2 to 2.5, or from 1.4 to 2.1 units from the PZC of the adjuvant.

The compositions may have a pH of at least 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, or 2.9 units from the PZC of the AlPO₄ adjuvant.

In some embodiments, AlPO₄ adjuvants of the disclosure are used in compositions having a pH such that the difference between the PZC of the AlPO₄ adjuvant and a pH of the composition is ranging from about 1.0 to about 2.9, or from about 1.2 to about 2.9.

The compositions may have a pH of at least 1.2 units from the PZC of the AlPO₄ adjuvant.

The compositions may have a pH of at no more 2.9 units from the PZC of the AlPO₄ adjuvant.

The compositions may have a pH of 1.2, 1.3, 1.4, 1.5, 1.6, 1.7, 1.8, 1.9, 2.0, 2.1, 2.2, 2.3, 2.4, 2.5, 2.6, 2.7, 2.8, or 2.9 units from the PZC of the AlPO₄ adjuvant.

The compositions may have a pH of 1.2, 1.3, 1.4, 1.5, 1.6, 1.7, 1.8, 1.9, 2.0, 2.1, 2.2, 2.3, 2.4, 2.5, 2.6, or 2.7 units from the PZC of the AlPO₄ adjuvant.

The compositions may have a pH of 1.2, 1.5, 1.7, 2.2, 2.5 or 2.7 units from the PZC of the AlPO₄ adjuvant.

The compositions may have a pH of 1.2, 1.3, 1.4, 1.5, 1.6 or 1.7 units from the PZC of the AlPO₄ adjuvant.

The compositions may have a pH of 1.2, 1.5 or 1.7 units from the PZC of the AlPO₄ adjuvant.

The compositions may have a pH from about 5.5 to about 7.0. The compositions may have a pH of about 5.5, 5.6, 5.7, 5.8, 5.9, 6.0, 6.1, 6.2, 6.3, 6.4, 6.5, 6.6, 6.7, 6.8, 6.9, or of about 7.0.

The compositions may have a pH of about 5.5, 6.0, 6.5, or about 7.0.

The compositions may have a pH of about 6.0.

The AlPO₄ adjuvant may have a PZC of about 4.5.

The compositions may have a pH within 1.5 units from the PZC of the AlPO₄ adjuvant.

The composition may comprise or consist of about 25 to about 100 μg/dose of a non-lipidated fHBP A protein comprising or consisting of SEQ ID NO: 2, about 25 to about 100 μg/dose of a non-lipidated fHBP B protein comprising or consisting of SEQ ID NO: 4, about 25 to about 100 μg/dose of a NadA protein comprising or consisting of SEQ ID NO: 5, about 20 to about 150 μg/dose of dOMV from a MenB strain expressing PorA VR2 P1.2, about 100 to about 600 μg/dose of aluminum phosphate adjuvant, 50 mM acetate buffer and pH 6.0.

The composition may comprise or consist of about 25 to about 100 μg/dose of a non-lipidated fHBP A protein comprising or consisting of SEQ ID NO: 8, about 25 to about 100 μg/dose of a non-lipidated fHBP B protein comprising or consisting of SEQ ID NO: 9, about 25 to about 100 μg/dose of a NadA protein comprising or consisting of SEQ ID NO: 5, about 20 to about 150 μg/dose of dOMV from a MenB strain expressing PorA VR2 P1.2, about 100 to about 600 μg/dose of aluminum phosphate adjuvant, 50 mM acetate buffer and pH 6.0.

The composition may further comprise at least a conjugated capsular saccharide from one or more of Neisseria meningitidis serogroups A, C, W135 and/or Y.

It is also disclosed a vaccine comprising a composition as described herein.

A composition or a vaccine as disclosed herein may be for use in protecting against a meningococcal infection or may be for use in inducing an immune response against a meningococcus bacterium.

It is further disclosed a composition comprising or consisting of a mRNA coding for an fHBP A protein comprising at least about 85%, at least about 90%, at least 95%, at least about 98%, at least about 99%, at least about 99.5%, or about 100% amino acid sequence identity to SEQ ID NO: 2, an mRNA encoding an fHBP B protein comprising at least about 85%, at least about 90%, at least about 95%, at least about 98%, at least about 99%, at least about 99.5%, or about 100% amino acid sequence identity to SEQ ID NO: 4, a mRNA coding for a NadA protein comprising at least about 85%, at least about 90%, at least about 95%, at least about 98%, at least about 99%, at least about 99.5%, or about 100% amino acid sequence identity to SEQ ID NO: 5 and a dOMV from a MenB expressing a PorA VR2 P1.2.

It is further disclosed a composition comprising or consisting of a mRNA coding for an fH-BP A protein comprising at least about 85%, at least about 90%, at least 95%, at least about 98%, at least about 99%, at least about 99.5%, or about 100% amino acid sequence identity to SEQ ID NO: 8, an mRNA encoding an fHBP B protein comprising at least about 85%, at least about 90%, at least about 95%, at least about 98%, at least about 99%, at least about 99.5%, or about 100% amino acid sequence identity to SEQ ID NO: 9, a mRNA coding for a NadA protein comprising at least about 85%, at least about 90%, at least about 95%, at least about 98%, at least about 99%, at least about 99.5%, or about 100% amino acid sequence identity to SEQ ID NO: 5 and a dOMV from a MenB expressing a PorA VR2 P1.2

### Formulations

An immunogenic composition as disclosed herein may be formulated into preparations in solid, semi-solid, liquid forms, such as tablets, capsules, powders, aerosols, solutions, suspensions, or emulsions. Actual methods of preparing such dosage forms are known, or will be apparent, to those skilled in this art; for example, see Remington: The Science and Practice of Pharmacy, 20th Edition (Philadelphia College of Pharmacy and Science, 2000). Compositions are formulated based upon the mode of delivery, including, for example, compositions may be formulated for delivery via parenteral delivery, such as intramuscular, intradermal, or subcutaneous injection.

An immunogenic composition may be administered via any suitable route, such as by mucosal administration (e.g., intranasal or sublingual), parenteral administration (e.g., intramuscular, subcutaneous, transcutaneous, or intradermal route), or oral administration. Typical routes of administering such compositions include, without limitation, oral, topical, transdermal, inhalation, parenteral, sublingual, buccal, intranasal. The term parenteral as used herein includes subcutaneous injections, intravenous, intramuscular, intradermal, intrasternal injection or infusion techniques. In some embodiments, a composition may be administered by transdermal, subcutaneous, intradermal or intramuscular route.

In one embodiment, an immunogenic composition as disclosed herein may be formulated to be administered via the intramuscular route, or the intradermal route, or the subcutaneous route. In one embodiment, an immunogenic composition may be formulated to be administered via the intramuscular route.

Immunogenic compositions may be formulated with any pharmaceutically acceptable excipient. The compositions may comprise at least one inert diluent or carrier. One exemplary pharmaceutically acceptable vehicle is a physiological saline buffer. Other physiologically acceptable vehicles are known to those skilled in the art and are described, for instance, in Remington's Pharmaceutical Sciences (18th edition), ed. A. Gennaro, 1990, Mack Publishing Company, Easton, Pa. An immunogenic composition as described herein may optionally comprise pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, human serum albumin, essential amino acids, nonessential amino acids, L-arginine hydrochlorate, saccharose, D-trehalose dehydrate, sorbitol, tris (hydroxymethyl) aminomethane and/or urea. In addition, the vaccine composition may optionally comprise pharmaceutically acceptable additives including, for example, diluents, binders, stabilizers, and preservatives.

A composition may be in the form of a liquid, for example, a solution, an emulsion or a suspension, intended to be for delivery by injection. Compositions intended to be administered by injection may comprise at least one of: a surfactant, preservative, wetting agent, dispersing agent, suspending agent, buffer, stabilizer and isotonic agent may be included. The liquid compositions as disclosed herein may include at least one of: sterile diluents such as water for injection, saline solution, such as physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; agents to act as cryoprotectants such as sucrose or trehalose.

In another embodiment, a composition of the disclosure may have a pH in a range of about 5.5 to about 7.0.

The pH of an immunogenic composition disclosed herein may range from about 5.5 to about 7.0, or from about 5.6 to about 6.9, or from about 5.7 to about 6.7, or from about 5.8 to about 6.5, or from about 5.9 to about 6.3. In one embodiment, a pH of a composition as disclosed herein may be about 6.0. Stable pH may be maintained by the use of a buffer.

In one embodiment, a composition of the disclosure may further comprise a buffer. As possible usable buffers, one may cite a Tris buffer, an acetate buffer, a citrate buffer, a phosphate buffer, an HEPES buffer, or a histidine buffer.

A composition may comprise a sodium acetate buffer.

A sodium acetate buffer may be present at a at a concentration ranging from about 10 mM to about 300 mM, or ranging from about 10 mM to about 250 mM, or ranging from about 20 mM to about 250 mM, or ranging from about 20 mM to about 150 mM, or from about 20 mM to about 130 mM, or from about 30 mM to about 120 mM, or from about 40 mM to about 100 mM, or from about 50 mM to about 80 mM, or from about 50 mM to about 60 mM, or for example at a concentration of about 50 mM.

Immunogenic compositions may be isotonic with respect to mammals, such as humans.

An immunogenic composition may also comprise one or several additional salts, such as a sodium salt, a calcium salt, or a magnesium salt. A sodium salt may be selected in the group comprising sodium chloride, sodium phosphate. A sodium salt may be sodium chloride. A calcium salt may be a calcium chloride salt. A magnesium salt may be a magnesium chloride salt.

A sodium salt may be present in a concentration ranging from about 10 mM to about 300 mM, or from about 30 mM to about 280 mM, or from about 50 mM to about 250 mM, or from about 60 mM to about 220 mM, or from about 80 mM to about 200 mM, or from about 100 mM to about 180 mM, or from about 120 mM to about 160 mM, or may be for example at a concentration of about 150 mM.

A calcium or a magnesium may be present in an amount ranging from about 1 mM to about 15 mM, or from about 5 mM to about 10 mM.

An immunogenic composition for parenteral administration can be enclosed in ampoules, disposable syringes or multiple dose vials made of glass or plastic. An injectable composition is for example sterile.

Immunogenic compositions may be sterilized by conventional sterilization techniques, for example with UV or gamma-radiation, or may be sterile filtered. The compositions obtained after sterile filtration may be packaged and stored in liquid form or in lyophilized from. A lyophilized composition may be reconstituted with a sterile aqueous carrier prior to administration. Dry compositions may include stabilizers such as mannitol, sucrose, or dodecyl maltoside, as well as mixtures thereof e.g., lactose/sucrose mixtures, sucrose/mannitol mixtures, etc.

The compositions as disclosed herein are administered to an individual in need thereof in a therapeutically effective amount, which will vary depending on a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the specific disorder or condition; and the subject undergoing therapy.

An immunogenic composition of the disclosure may comprise or consist of:
a non-lipidated mutated fHBP A comprising or consisting of SEQ ID NO: 2, a non-lipidated mutated fHBP B comprising or consisting of SEQ ID NO: 4, a NadA protein comprising or consisting of SEQ ID NO: 5, dOMV from a MenB expressing PorA VR2 P1.2, and an AlPO₄ adjuvant selected as having a PZC of about 4.3. The composition may comprise a 50 mM acetate buffer and pH 6.0, or
a non-lipidated mutated fHBP A consisting of SEQ ID NO: 2, a non-lipidated mutated fHBP B consisting of SEQ ID NO: 4, a NadA protein consisting of SEQ ID NO: 5, dOMV from a MenB expressing PorA VR2 P1.2, an AlPO₄ adjuvant selected as having a PZC of about 4.3, 50 mM acetate buffer and pH 6.0, or
about 25 to about 100 μg/dose of a non-lipidated mutated fHBP A consisting of SEQ ID NO: 2, about 25 to about 100 μg/dose of a non-lipidated mutated fHBP B consisting of SEQ ID NO: 4, about 25 to about 100 μg/dose of a NadA consisting of SEQ ID NO: 5, about 20 to about 250 μg/dose of dOMV from a MenB strain expressing PorA VR2 P1.2, about 100 to about 800 μg/dose of an AlPO₄ adjuvant selected as having a PZC of about 4.3, 50 mM acetate buffer and pH 6.0, or
about 25 μg/dose of a non-lipidated mutated fHBP A consisting of SEQ ID NO: 2, about 25 μg/dose of a non-lipidated mutated fHBP B consisting of SEQ ID NO: 4, about 25 μg/dose of a NadA protein consisting of SEQ ID NO: 5, about 25 μg/dose of dOMV from a MenB strain expressing PorA VR2 P1.2, about 100 μg/dose of an AlPO₄ adjuvant selected as having a PZC of about 4.3, 50 mM acetate buffer and pH 6.0, or
about 25 μg/dose of a non-lipidated mutated fHBP A consisting of SEQ ID NO: 2, about 25 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant of PZC of about 4.3, 50 mM acetate buffer and pH 6.0, or
about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 25 μg/dose of dOMV from a MenB strain expressing PorA VR2 P1.2, about 400 μg/dose of an AlPO₄ adjuvant selected as having a PZC of about 4.3, 50 mM acetate buffer and pH 6.0, or
about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant selected as having a PZC of about 4.3, 50 mM acetate buffer and pH 6.0, or
about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.3, 50 mM acetate buffer and pH 6.0, or
about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 200 μg/dose of an AlPO₄ adjuvant selected as having a PZC of about 4.3, 50 mM acetate buffer and pH 6.0, or
about 75 μg/dose of a non-lipidated mutated fHBP A consisting of SEQ ID NO: 2, about 75 μg/dose of a non-lipidated mutated fHBP B consisting of SEQ ID NO: 4, about 75 μg/dose of a NadA protein consisting of SEQ ID NO: 5, about 75 μg/dose of dOMV from a MenB strain expressing PorA VR2 P1.2, about 300 μg/dose of an AlPO₄ adjuvant selected as having a PZC of about 4.3, 50 mM acetate buffer and pH 6.0, or
about 100 μg/dose of a non-lipidated mutated fHBP A consisting of SEQ ID NO: 2, about 100 μg/dose of a non-lipidated mutated fHBP B consisting of SEQ ID NO: 4, about 100 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.3, 50 mM acetate buffer and pH 6.0, or
about 100 μg/dose of a non-lipidated mutated fHBP A consisting of SEQ ID NO: 2, about 100 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant of PZC of about 4.3, 50 mM acetate buffer and pH 6.0, or
about 100 μg/dose of a non-lipidated mutated fHBP A consisting of SEQ ID NO: 2, about 100 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 800 μg/dose of an AlPO₄ adjuvant selected as having a PZC of about 4.3, 50 mM acetate buffer and pH 6.0.

In one embodiment, an immunogenic composition may comprise or consist of about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.3, 50 mM acetate buffer and pH 6.0.

An immunogenic composition of the disclosure may comprise or consist of:
a non-lipidated mutated fHBP A comprising or consisting of SEQ ID NO: 2, a non-lipidated mutated fHBP B comprising or consisting of SEQ ID NO: 4, a NadA protein comprising or consisting of SEQ ID NO: 5, dOMV from a MenB expressing PorA VR2 P1.2, and an AlPO₄ adjuvant selected as having a PZC of about 4.5. The composition may comprise a 50 mM acetate buffer and pH 6.0, or
a non-lipidated mutated fHBP A consisting of SEQ ID NO: 2, a non-lipidated mutated fHBP B consisting of SEQ ID NO: 4, a NadA protein consisting of SEQ ID NO: 5, dOMV from a MenB expressing PorA VR2 P1.2, an AlPO₄ adjuvant selected as having a PZC of about 4.5, 50 mM acetate buffer and pH 6.0, or
about 25 to about 100 μg/dose of a non-lipidated mutated fHBP A consisting of SEQ ID NO: 2, about 25 to about 100 μg/dose of a non-lipidated mutated fHBP B consisting of SEQ ID NO: 4, about 25 to about 100 μg/dose of a NadA consisting of SEQ ID NO: 5, about 20 to about 250 μg/dose of dOMV from a MenB strain expressing PorA VR2 P1.2, about 100 to about 800 μg/dose of an AlPO₄ adjuvant selected as having a PZC of about 4.5, 50 mM acetate buffer and pH 6.0, or
about 25 μg/dose of a non-lipidated mutated fHBP A consisting of SEQ ID NO: 2, about 25 μg/dose of a non-lipidated mutated fHBP B consisting of SEQ ID NO: 4, about 25 μg/dose of a NadA protein consisting of SEQ ID NO: 5, about 25 μg/dose of dOMV from a MenB strain expressing PorA VR2 P1.2, about 100 μg/dose of an AlPO₄ adjuvant selected as having a PZC of about 4.5, 50 mM acetate buffer and pH 6.0, or
about 25 μg/dose of a non-lipidated mutated fHBP A consisting of SEQ ID NO: 2, about 25 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant of PZC of about 4.5, 50 mM acetate buffer and pH 6.0, or
about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 25 μg/dose of dOMV from a MenB strain expressing PorA VR2 P1.2, about 400 μg/dose of an AlPO₄ adjuvant selected as having a PZC of about 4.5, 50 mM acetate buffer and pH 6.0, or
about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant selected as having a PZC of about 4.5, 50 mM acetate buffer and pH 6.0, or
about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.5, 50 mM acetate buffer and pH 6.0, or
about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 200 μg/dose of an AlPO₄ adjuvant selected as having a PZC of about 4.5, 50 mM acetate buffer and pH 6.0, or
about 75 μg/dose of a non-lipidated mutated fHBP A consisting of SEQ ID NO: 2, about 75 μg/dose of a non-lipidated mutated fHBP B consisting of SEQ ID NO: 4, about 75 μg/dose of a NadA protein consisting of SEQ ID NO: 5, about 75 μg/dose of dOMV from a MenB strain expressing PorA VR2 P1.2, about 300 μg/dose of an AlPO₄ adjuvant selected as having a PZC of about 4.5, 50 mM acetate buffer and pH 6.0, or
about 100 μg/dose of a non-lipidated mutated fHBP A consisting of SEQ ID NO: 2, about 100 μg/dose of a non-lipidated mutated fHBP B consisting of SEQ ID NO: 4, about 100 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.5, 50 mM acetate buffer and pH 6.0, or
about 100 μg/dose of a non-lipidated mutated fHBP A consisting of SEQ ID NO: 2, about 100 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant of PZC of about 4.5, 50 mM acetate buffer and pH 6.0, or
about 100 μg/dose of a non-lipidated mutated fHBP A consisting of SEQ ID NO: 2, about 100 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 800 μg/dose of an AlPO₄ adjuvant selected as having a PZC of about 4.5, 50 mM acetate buffer and pH 6.0.

In one embodiment, an immunogenic composition may comprise or consist of about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.5, 50 mM acetate buffer and pH 6.0.

An immunogenic composition of the disclosure may comprise or consist of:
a non-lipidated mutated fHBP A comprising or consisting of SEQ ID NO: 2, a non-lipidated mutated fHBP B comprising or consisting of SEQ ID NO: 4, a NadA protein comprising or consisting of SEQ ID NO: 5, dOMV from a MenB expressing PorA VR2 P1.2, and an AlPO₄ adjuvant selected as having a PZC of about 4.8. The composition may comprise a 50 mM acetate buffer and pH 6.0, or
a non-lipidated mutated fHBP A consisting of SEQ ID NO: 2, a non-lipidated mutated fHBP B consisting of SEQ ID NO: 4, a NadA protein consisting of SEQ ID NO: 5, dOMV from a MenB expressing PorA VR2 P1.2, an AlPO₄ adjuvant selected as having a PZC of about 4.8, 50 mM acetate buffer and pH 6.0, or
about 25 to about 100 μg/dose of a non-lipidated mutated fHBP A consisting of SEQ ID NO: 2, about 25 to about 100 μg/dose of a non-lipidated mutated fHBP B consisting of SEQ ID NO: 4, about 25 to about 100 μg/dose of a NadA consisting of SEQ ID NO: 5, about 20 to about 250 μg/dose of dOMV from a MenB strain expressing PorA VR2 P1.2, about 100 to about 800 μg/dose of an AlPO₄ adjuvant selected as having a PZC of about 4.8, 50 mM acetate buffer and pH 6.0, or
about 25 μg/dose of a non-lipidated mutated fHBP A consisting of SEQ ID NO: 2, about 25 μg/dose of a non-lipidated mutated fHBP B consisting of SEQ ID NO: 4, about 25 μg/dose of a NadA protein consisting of SEQ ID NO: 5, about 25 μg/dose of dOMV from a MenB strain expressing PorA VR2 P1.2, about 100 μg/dose of an AlPO₄ adjuvant selected as having a PZC of about 4.8, 50 mM acetate buffer and pH 6.0, or
about 25 μg/dose of a non-lipidated mutated fHBP A consisting of SEQ ID NO: 2, about 25 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant of PZC of about 4.8, 50 mM acetate buffer and pH 6.0, or
about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 25 μg/dose of dOMV from a MenB strain expressing PorA VR2 P1.2, about 400 μg/dose of an AlPO₄ adjuvant selected as having a PZC of about 4.8, 50 mM acetate buffer and pH 6.0, or
about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant selected as having a PZC of about 4.8, 50 mM acetate buffer and pH 6.0, or
about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.8, 50 mM acetate buffer and pH 6.0, or
about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 200 μg/dose of an AlPO₄ adjuvant selected as having a PZC of about 4.8, 50 mM acetate buffer and pH 6.0, or
about 75 μg/dose of a non-lipidated mutated fHBP A consisting of SEQ ID NO: 2, about 75 μg/dose of a non-lipidated mutated fHBP B consisting of SEQ ID NO: 4, about 75 μg/dose of a NadA protein consisting of SEQ ID NO: 5, about 75 μg/dose of dOMV from a MenB strain expressing PorA VR2 P1.2, about 300 μg/dose of an AlPO₄ adjuvant selected as having a PZC of about 4.8, 50 mM acetate buffer and pH 6.0, or
about 100 μg/dose of a non-lipidated mutated fHBP A consisting of SEQ ID NO: 2, about 100 μg/dose of a non-lipidated mutated fHBP B consisting of SEQ ID NO: 4, about 100 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.8, 50 mM acetate buffer and pH 6.0, or
about 100 μg/dose of a non-lipidated mutated fHBP A consisting of SEQ ID NO: 2, about 100 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant of PZC of about 4.8, 50 mM acetate buffer and pH 6.0, or
of about 100 μg/dose of a non-lipidated mutated fHBP A consisting of SEQ ID NO: 2, about 100 μg/dose of a non-lipidated mutated fHBP B consisting of SEQ ID NO: 4, about 50 μg/dose of a NadA protein consisting of SEQ ID NO: 5, about 50 μg/dose of dOMV from a MenB strain expressing PorA VR2 P1.2, about 800 μg/dose of an AlPO₄ adjuvant selected as having a PZC of about 4.8, 50 mM acetate buffer and pH 6.0.

In one embodiment, an immunogenic composition may comprise or consist of about 50 μg/dose of a non-lipidated mutated fHBP A consisting of SEQ ID NO: 2, about 50 μg/dose of a non-lipidated mutated fHBP B consisting of SEQ ID NO: 4, about 50 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.8, 50 mM acetate buffer and pH 6.0.

An immunogenic composition of the disclosure may comprise or consist of:
a non-lipidated mutated fHBP A comprising or consisting of SEQ ID NO: 8, a non-lipidated mutated fHBP B comprising or consisting of SEQ ID NO: 9, a NadA protein comprising or consisting of SEQ ID NO: 5, dOMV from a MenB expressing PorA VR2 P1.2, and an AlPO₄ adjuvant selected as having a PZC of about 4.3. The composition may comprise a 50 mM acetate buffer and pH 6.0, or
a non-lipidated mutated fHBP A consisting of SEQ ID NO: 8, a non-lipidated mutated fHBP B consisting of SEQ ID NO: 9, a NadA protein consisting of SEQ ID NO: 5, dOMV from a MenB expressing PorA VR2 P1.2, an AlPO₄ adjuvant selected as having a PZC of about 4.3, 50 mM acetate buffer and pH 6.0, or
about 25 to about 100 μg/dose of a non-lipidated mutated fHBP A consisting of SEQ ID NO: 8, about 25 to about 100 μg/dose of a non-lipidated mutated fHBP B consisting of SEQ ID NO: 9, about 25 to about 100 μg/dose of a NadA consisting of SEQ ID NO: 5, about 20 to about 250 μg/dose of dOMV from a MenB strain expressing PorA VR2 P1.2, about 100 to about 800 μg/dose of an AlPO₄ adjuvant selected as having a PZC of about 4.3, 50 mM acetate buffer and pH 6.0, or
about 25 μg/dose of a non-lipidated mutated fHBP A consisting of SEQ ID NO: 8, about 25 μg/dose of a non-lipidated mutated fHBP B consisting of SEQ ID NO: 9, about 25 μg/dose of a NadA protein consisting of SEQ ID NO: 5, about 25 μg/dose of dOMV from a MenB strain expressing PorA VR2 P1.2, about 100 μg/dose of an AlPO₄ adjuvant selected as having a PZC of about 4.3, 50 mM acetate buffer and pH 6.0, or
about 25 μg/dose of a non-lipidated mutated fHBP A consisting of SEQ ID NO: 8, about 25 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant of PZC of about 4.3, 50 mM acetate buffer and pH 6.0, or
about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 25 μg/dose of dOMV from a MenB strain expressing PorA VR2 P1.2, about 400 μg/dose of an AlPO₄ adjuvant selected as having a PZC of about 4.3, 50 mM acetate buffer and pH 6.0, or
about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant selected as having a PZC of about 4.3, 50 mM acetate buffer and pH 6.0, or
about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.3, 50 mM acetate buffer and pH 6.0, or
about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 200 μg/dose of an AlPO₄ adjuvant selected as having a PZC of about 4.3, 50 mM acetate buffer and pH 6.0, or
about 75 μg/dose of a non-lipidated mutated fHBP A consisting of SEQ ID NO: 8, about 75 μg/dose of a non-lipidated mutated fHBP B consisting of SEQ ID NO: 9, about 75 μg/dose of a NadA protein consisting of SEQ ID NO: 5, about 75 μg/dose of dOMV from a MenB strain expressing PorA VR2 P1.2, about 300 μg/dose of an AlPO₄ adjuvant selected as having a PZC of about 4.3, 50 mM acetate buffer and pH 6.0, or
about 100 μg/dose of a non-lipidated mutated fHBP A consisting of SEQ ID NO: 8, about 100 μg/dose of a non-lipidated mutated fHBP B consisting of SEQ ID NO: 9, about 100 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.3, 50 mM acetate buffer and pH 6.0, or
about 100 μg/dose of a non-lipidated mutated fHBP A consisting of SEQ ID NO: 8, about 100 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant of PZC of about 4.3, 50 mM acetate buffer and pH 6.0, or
about 100 μg/dose of a non-lipidated mutated fHBP A consisting of SEQ ID NO: 8, about 100 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 800 μg/dose of an AlPO₄ adjuvant selected as having a PZC of about 4.3, 50 mM acetate buffer and pH 6.0.

In one embodiment, an immunogenic composition may comprise or consist of about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.3, 50 mM acetate buffer and pH 6.0.

An immunogenic composition of the disclosure may comprise or consist of:
a non-lipidated mutated fHBP A comprising or consisting of SEQ ID NO: 8, a non-lipidated mutated fHBP B comprising or consisting of SEQ ID NO: 9, a NadA protein comprising or consisting of SEQ ID NO: 5, dOMV from a MenB expressing PorA VR2 P1.2, and an AlPO₄ adjuvant selected as having a PZC of about 4.5. The composition may comprise a 50 mM acetate buffer and pH 6.0, or
a non-lipidated mutated fHBP A consisting of SEQ ID NO: 8, a non-lipidated mutated fHBP B consisting of SEQ ID NO: 9, a NadA protein consisting of SEQ ID NO: 5, dOMV from a MenB expressing PorA VR2 P1.2, an AlPO₄ adjuvant selected as having a PZC of about 4.5, 50 mM acetate buffer and pH 6.0, or
about 25 to about 100 μg/dose of a non-lipidated mutated fHBP A consisting of SEQ ID NO: 8, about 25 to about 100 μg/dose of a non-lipidated mutated fHBP B consisting of SEQ ID NO: 9, about 25 to about 100 μg/dose of a NadA consisting of SEQ ID NO: 5, about 20 to about 250 μg/dose of dOMV from a MenB strain expressing PorA VR2 P1.2, about 100 to about 800 μg/dose of an AlPO₄ adjuvant selected as having a PZC of about 4.5, 50 mM acetate buffer and pH 6.0, or
about 25 μg/dose of a non-lipidated mutated fHBP A consisting of SEQ ID NO: 8, about 25 μg/dose of a non-lipidated mutated fHBP B consisting of SEQ ID NO: 9, about 25 μg/dose of a NadA protein consisting of SEQ ID NO: 5, about 25 μg/dose of dOMV from a MenB strain expressing PorA VR2 P1.2, about 100 μg/dose of an AlPO₄ adjuvant selected as having a PZC of about 4.5, 50 mM acetate buffer and pH 6.0, or
about 25 μg/dose of a non-lipidated mutated fHBP A consisting of SEQ ID NO: 8, about 25 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant of PZC of about 4.5, 50 mM acetate buffer and pH 6.0, or
about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 25 μg/dose of dOMV from a MenB strain expressing PorA VR2 P1.2, about 400 μg/dose of an AlPO₄ adjuvant selected as having a PZC of about 4.5, 50 mM acetate buffer and pH 6.0, or
about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant selected as having a PZC of about 4.5, 50 mM acetate buffer and pH 6.0, or
about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.5, 50 mM acetate buffer and pH 6.0, or
about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 200 μg/dose of an AlPO₄ adjuvant selected as having a PZC of about 4.5, 50 mM acetate buffer and pH 6.0, or
about 75 μg/dose of a non-lipidated mutated fHBP A consisting of SEQ ID NO: 8, about 75 μg/dose of a non-lipidated mutated fHBP B consisting of SEQ ID NO: 9, about 75 μg/dose of a NadA protein consisting of SEQ ID NO: 5, about 75 μg/dose of dOMV from a MenB strain expressing PorA VR2 P1.2, about 300 μg/dose of an AlPO₄ adjuvant selected as having a PZC of about 4.5, 50 mM acetate buffer and pH 6.0, or
about 100 μg/dose of a non-lipidated mutated fHBP A consisting of SEQ ID NO: 8, about 100 μg/dose of a non-lipidated mutated fHBP B consisting of SEQ ID NO: 9, about 100 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.5, 50 mM acetate buffer and pH 6.0, or
about 100 μg/dose of a non-lipidated mutated fHBP A consisting of SEQ ID NO: 8, about 100 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant of PZC of about 4.5, 50 mM acetate buffer and pH 6.0, or
about 100 μg/dose of a non-lipidated mutated fHBP A consisting of SEQ ID NO: 8, about 100 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 800 μg/dose of an AlPO₄ adjuvant selected as having a PZC of about 4.5, 50 mM acetate buffer and pH 6.0.

In one embodiment, an immunogenic composition may comprise or consist of about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.5, 50 mM acetate buffer and pH 6.0.

An immunogenic composition of the disclosure may comprise or consist of:
a non-lipidated mutated fHBP A comprising or consisting of SEQ ID NO: 8, a non-lipidated mutated fHBP B comprising or consisting of SEQ ID NO: 9, a NadA protein comprising or consisting of SEQ ID NO: 5, dOMV from a MenB expressing PorA VR2 P1.2, and an AlPO₄ adjuvant selected as having a PZC of about 4.8. The composition may comprise a 50 mM acetate buffer and pH 6.0, or
a non-lipidated mutated fHBP A consisting of SEQ ID NO: 8, a non-lipidated mutated fHBP B consisting of SEQ ID NO: 9, a NadA protein consisting of SEQ ID NO: 5, dOMV from a MenB expressing PorA VR2 P1.2, an AlPO₄ adjuvant selected as having a PZC of about 4.8, 50 mM acetate buffer and pH 6.0, or
about 25 to about 100 μg/dose of a non-lipidated mutated fHBP A consisting of SEQ ID NO: 8, about 25 to about 100 μg/dose of a non-lipidated mutated fHBP B consisting of SEQ ID NO: 9, about 25 to about 100 μg/dose of a NadA consisting of SEQ ID NO: 5, about 20 to about 250 μg/dose of dOMV from a MenB strain expressing PorA VR2 P1.2, about 100 to about 800 μg/dose of an AlPO₄ adjuvant selected as having a PZC of about 4.8, 50 mM acetate buffer and pH 6.0, or
about 25 μg/dose of a non-lipidated mutated fHBP A consisting of SEQ ID NO: 8, about 25 μg/dose of a non-lipidated mutated fHBP B consisting of SEQ ID NO: 9, about 25 μg/dose of a NadA protein consisting of SEQ ID NO: 5, about 25 μg/dose of dOMV from a MenB strain expressing PorA VR2 P1.2, about 100 μg/dose of an AlPO₄ adjuvant selected as having a PZC of about 4.8, 50 mM acetate buffer and pH 6.0, or
about 25 μg/dose of a non-lipidated mutated fHBP A consisting of SEQ ID NO: 8, about 25 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant of PZC of about 4.8, 50 mM acetate buffer and pH 6.0, or
about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 25 μg/dose of dOMV from a MenB strain expressing PorA VR2 P1.2, about 400 μg/dose of an AlPO₄ adjuvant selected as having a PZC of about 4.8, 50 mM acetate buffer and pH 6.0, or
about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant selected as having a PZC of about 4.8, 50 mM acetate buffer and pH 6.0, or
about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.8, 50 mM acetate buffer and pH 6.0, or
about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 200 μg/dose of an AlPO₄ adjuvant selected as having a PZC of about 4.8, 50 mM acetate buffer and pH 6.0, or
about 75 μg/dose of a non-lipidated mutated fHBP A consisting of SEQ ID NO: 8, about 75 μg/dose of a non-lipidated mutated fHBP B consisting of SEQ ID NO: 9, about 75 μg/dose of a NadA protein consisting of SEQ ID NO: 5, about 75 μg/dose of dOMV from a MenB strain expressing PorA VR2 P1.2, about 300 μg/dose of an AlPO₄ adjuvant selected as having a PZC of about 4.8, 50 mM acetate buffer and pH 6.0, or
about 100 μg/dose of a non-lipidated mutated fHBP A consisting of SEQ ID NO: 8, about 100 μg/dose of a non-lipidated mutated fHBP B consisting of SEQ ID NO: 9, about 100 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.8, 50 mM acetate buffer and pH 6.0, or
about 100 μg/dose of a non-lipidated mutated fHBP A consisting of SEQ ID NO: 8, about 100 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 400 μg/dose of an AlPO₄ adjuvant of PZC of about 4.8, 50 mM acetate buffer and pH 6.0, or
of about 100 μg/dose of a non-lipidated mutated fHBP A consisting of SEQ ID NO: 8, about 100 μg/dose of a non-lipidated mutated fHBP B consisting of SEQ ID NO: 9, about 50 μg/dose of a NadA protein consisting of SEQ ID NO: 5, about 50 μg/dose of dOMV from a MenB strain expressing PorA VR2 P1.2, about 800 μg/dose of an AlPO₄ adjuvant selected as having a PZC of about 4.8, 50 mM acetate buffer and pH 6.0.

In one embodiment, an immunogenic composition may comprise or consist of about 50 μg/dose of a non-lipidated mutated fHBP A consisting of SEQ ID NO: 8, about 50 μg/dose of a non-lipidated mutated fHBP B consisting of SEQ ID NO: 9, about 50 μg/dose of a NadA protein consisting of SEQ ID NO: 5, about 125 μg/dose of dOMV from a MenB strain expressing PorA VR2 P1.2, about 400 μg/dose of an AlPO₄ adjuvant selected as having a PZC of about 4.8, 50 mM acetate buffer and pH 6.0.

A dose may range from about 0.1 mL to about 1 mL, for example from about 0.2 mL to about 0.8 mL, from about 0.4 mL to about 0.6 mL, or may be of about 0.5 mL.

In one embodiment, the present disclosure relates to a container comprising a composition as disclosed herein. A container may comprise an immunogenic composition comprising a combination of meningococcal antigens, said combination comprising at least one factor H binding protein (fHBP) A, at least one fHBP B, and an aluminum hydroxyphosphate (AlPO₄) adjuvant, the AlPO₄ adjuvant being selected as having a point of zero charge (PZC) below 5.

The container may further comprise at least one Neisseria adhesin A (NadA) protein and/or at least one detergent-extracted Outer Membrane Vesicle (dOMV).

The container may comprise a composition of the disclosure as previously detailed.

A container may comprise a composition comprising or consisting of from about 25 to 100 μg/dose of a non-lipidated mutated fHBP A consisting of SEQ ID NO: 2, from about 25 to 100 μg/dose of a non-lipidated mutated fHBP B consisting of SEQ ID NO: 4, from about 25 to 100 μg/dose of a NadA protein consisting of SEQ ID NO: 5, from about 20 to 250 μg/dose of dOMV from a MenB strain expressing PorA VR2 P1.2, from about 100 to 800 μg/dose of an AlPO₄ adjuvant selected as having a point of zero charge (PZC) below 5, 50 mM acetate buffer and pH 6.0.

A container may comprise a composition comprising or consisting of from about 25 to 100 μg/dose of a non-lipidated mutated fHBP A consisting of SEQ ID NO: 8, from about 25 to 100 μg/dose of a non-lipidated mutated fHBP B consisting of SEQ ID NO: 9, from about 25 to 100 μg/dose of a NadA protein consisting of SEQ ID NO: 5, from about 20 to 250 μg/dose of dOMV from a MenB strain expressing PorA VR2 P1.2, from about 100 to 800 μg/dose of an AlPO₄ adjuvant selected as having a point of zero charge (PZC) below 5, 50 mM acetate buffer and pH 6.0.

In addition, a container may comprise a further antigen as above detailed. Alternatively, a further antigen may be packaged in a separate container.

A container may be a vial. A vial may be a multi-dose vials or may be a single-dose vial. Suitable vials may be a small glass or plastic container sealed with the most suitable stopper and seal.

Alternatively, a container may be a pre-filled syringe. A prefilled syringe may include a syringe barrel storing a liquid composition as disclosed herein. A gasket and a plunger are inserted in the syringe barrel. The gasket seals the syringe barrel in a liquid-tight manner to prevent leakage of the liquid drug, and the plunger slides the gasket. Various types of prefilled syringes are known in the art, as for example described in U.S. Pat. No. 10,625,025 or in WO 2013/046855.

In case where a composition of the disclosure is to be mixed and injected with another vaccine composition, as for example a tetravalent MenACWY-conjugated composition, both compositions may be packaged in a container being a single vial or a pre-filled syringe or in a dual-chamber syringe. A dual-chamber syringe also known as a sequential or bypass syringe, may comprise a single barrel separated by a septum into two compartments, proximal and distal. Depression of the syringe's plunger forces admixing of the two vaccine compositions in the distal compartment. Various types of dual-chamber syringes are known in the art, as for example described in U.S. Pat. No. 10,695,505. A dual-chamber syringe may be also used in case a vaccine composition is formulated in a dried-form, such as a lyophilized form, and is stored with the liquid vehicle for reconstitution. In such case, the dried vaccine is stored in one chamber and the liquid for reconstitution and injection is stored in a second chamber.

In some embodiments, the disclosure relates to a vaccine comprising an immunogenic composition as disclosed herein.

An immunogenic composition of the disclosure may be a vaccine.

### Kit-of-Parts

It is also disclosed kit-of-parts.

A kit-of-parts may comprise at least two containers, a first container comprising at least one factor H binding protein (fHBP) A and at least one factor H binding protein (fHBP) B, and a second container containing an aluminum hydroxyphosphate (AlPO₄) adjuvant selected for as having a point of zero charge (PZC) below 5.

A kit-of-parts may comprise at least a further container comprising at least one detergent-extracted Outer Membrane Vesicle (dOMV) and/or at least one Neisseria adhesin A (NadA) protein. The dOMV and the NadA protein may be provided in separate containers.

Alternatively, a kit-of-parts may comprise at least a first container comprising at least one factor H binding protein (fHBP) A, at least one factor H binding protein (fHBP) B, and an aluminum hydroxyphosphate (AlPO₄) adjuvant selected for as having a point of zero charge (PZC) below 5, and a second container comprising at least one detergent-extracted Outer Membrane Vesicle (dOMV) and/or at least one Neisseria adhesin A (NadA) protein. The dOMV and the NadA protein may be provided in separate containers.

Alternatively, a kit-of-parts may comprise at least a first container comprising at least one factor H binding protein (fHBP) A, at least one factor H binding protein (fHBP) B, an aluminum hydroxyphosphate (AlPO₄) adjuvant selected for as having a point of zero charge (PZC) below 5, at least one detergent-extracted Outer Membrane Vesicle (dOMV), and at least one Neisseria adhesin A (NadA) protein, and second container containing a further antigen.

Alternatively, each of the antigens, fHBP A, fHBP B, NadA and dOMV, and the AlPO₄ adjuvant selected for as having a point of zero charge (PZC) below 5 may be stored in a separate container. Alternately again, the antigens and the AlPO₄ adjuvant may be associated in different combinations. All type of combinations may be envisioned: fHBP A+B and NadA+dOMV and AlPO₄; fHBP A+B+AlPO₄ and NadA+dOMV; fHBP A+B+AlPO₄ and NadA+dOMV+AlPO₄; fHBP A+NadA and fHBP B+dOMV and AlPO₄; fHBP B+NadA+AlPO₄ and fHBP A+dOMV+AlPO₄; fHBP A+NadA+AlPO₄+fHBPB+AlPO₄ and dOMV+AlPO₄; or fHBP A+dOMV+fHBP B+AlPO₄ and NadA+AlPO₄; etc.

The fHBP A and B, the AlPO₄ adjuvant, the NadA protein and the dOMV are as above detailed.

In one embodiment, a further antigen may be a combination of conjugated MenACWY polysaccharides.

The conjugated MenACWY polysaccharides may be as above detailed.

In one embodiment, a kit-of-parts may comprise a first container comprising an immunogenic composition as disclosed herein and a second container comprising a combination of conjugated MenACWY polysaccharides.

The antigens and AlPO₄ of the immunogenic compositions of the disclosure may be prepared and stored in separate containers or vials. They then may be mixed at the time of the administration to an individual.

The antigens may be stored in liquid formulations or in a dried form. When formulated in dried form, a further container with an injectable liquid may be added, and the injectable liquid may be used to resuspend and mix the different antigens. A suitable injectable liquid carrier may comprise a buffer. The injectable liquid may comprise the AlPO₄ adjuvant.

In one embodiment, a container of the kit-of-parts may contain antigens may be in dried form. The antigens may be lyophilized in cake or as micropellets.

The kit may optionally comprise a container comprising a physiologically injectable vehicle. The physiologically injectable vehicle may be used resuspend or dissolve the antigens in dried form.

### Manufacturing Methods

The disclosure relates to a method for manufacturing an immunogenic composition comprising a combination of Neisseria meningitidis serogroup B antigens, said combination comprising at least one factor H binding protein (fHBP) A and one factor H binding protein (fHBP) B, and an AlPO₄ adjuvant, the method comprising at least the steps of:
a) selecting an AlPO₄ adjuvant having a PZC below 5, and
b) combining the AlPO₄ adjuvant selected at step a) with at least one factor H binding protein (fHBP) A and at least one factor H binding protein (fHBP) B, the combination being carried out in any order.

The method allows obtaining an immunogenic composition.

The combination of the AlPO₄ adjuvant with fHBP A and fHBP B may be carried out in any order. For example, the AlPO₄ adjuvant may be combined with fHBP A, and then fHBP B may be added, or the AlPO₄ adjuvant may be combined with fHBP B, and then fHBP A may be added, or fHBP A and fHBP B may be combined and then AlPO₄ may be added. or the AlPO₄ adjuvant may be combined with both fHBP A and fHBP B at the same time.

At step b), fHBP A and fHBP B may be first combined and then the AlPO₄ adjuvant may be added.

Alternatively, at step b), AlPO₄ adjuvant and fHBP A may be first combined and then fHBP B may be added.

Alternatively, at step b), AlPO₄ adjuvant and fHBP B may be first combined and then fHBP A may be added.

Alternatively, at step b), a first part of AlPO₄ adjuvant and fHBP B may be combined in a first mixture, and the second part of AlPO₄ adjuvant and fHBP A may be combined in a second mixture, and then the first and the second mixture may be combined.

Alternatively, at step b), the AlPO₄ adjuvant may be combined with both fHBP A and fHBP B at the same time.

The methods of the disclosure may further comprise a step of adding at least one antigen selected from NadA protein and dOMV. The combination may be carried out in any order.

For example, NadA proteins and/or a dOMV may be added before or after the step of combining AlPO₄ with fHBP A and fHBP B. NadA and dOMV may each be added in separate steps or may be combined before being added in a single step.

In some embodiments, at step b), fHBP A, fHBP B, NadA protein and dOMV may be combined in any order, and then the AlPO₄ adjuvant may be added.

In some embodiments, at step b), the AlPO₄ adjuvant may be fractioned in a plurality of fractions (2, 3 or 4) and a fraction may be added to each antigen: fHBP A, fHBP B, NadA protein and dOMV, and then the antigens with the AlPO₄ may be combined together in any order.

Alternatively, step b) may comprise combining the AlPO₄ adjuvant to a combination of fHBP A, fHBP B, and NadA protein. The dOMV may be added in a subsequent step.

Alternatively, step b) may comprise combining the AlPO₄ adjuvant to a combination of fHBP A, fHBP B, and dOMV. The NadA protein may be added in a subsequent step.

A method of manufacturing of an immunogenic composition of the disclosure may comprise at least the steps of:
a) selecting an AlPO₄ adjuvant having a PZC below 5,
b) combining the AlPO₄ adjuvant selected at step a) with at least one factor H binding protein (fHBP) A, at least one factor H binding protein (fHBP) B, at least one NadA protein, at least one dOMV, the combination being carried out in any order.

The Neisseria meningitidis antigens fHBP A, fHBP B, NadA protein, dOMV may be filtered, for example with a sterile filtration, for example with 0.22 μm filter, before combination with the AlPO₄ adjuvant.

Further to the combination of the AlPO₄ adjuvant, the fHBP A, the fHBP B, the NadA protein and the dOMV, the obtained combination may be then distributed in syringes or vials.

The disclosure relates to a method for preparing an immunogenic composition comprising a N. meningitidis fHBP B antigen, said composition inducing an enhanced an immune response against a N. meningitidis serogroup B strain expressing a fHBP B heterologous to said fHBP B antigen of said composition, the method comprising at least the steps of:
a) selecting an AlPO₄ adjuvant having a PZC below 5, and
b) combining the AlPO₄ adjuvant selected at step a) with said fHBP B, and
c) obtaining said immunogenic composition.

The method allows obtaining an immunogenic composition able to induce an enhanced an immune response against a N. meningitidis serogroup B strain expressing a fHBP B heterologous to said fHBP B of said composition.

According to another of its objects, the present disclosure relates to a method for preparing an immunogenic composition comprising a N. meningitidis fHBP B antigen, said composition inducing an enhanced immune response against a N. meningitidis serogroup B strain expressing a fHBP B homologous to said fHBP B antigen of said composition, the method comprising at least the steps of:
a) selecting an AlPO₄ adjuvant having a PZC below 5, and
b) combining the AlPO₄ adjuvant selected at step a) with said fHBP B, and
c) obtaining said immunogenic composition.

The method allows obtaining an immunogenic composition able to induce an enhanced an immune response against a N. meningitidis serogroup B strain expressing a fHBP B homologous to said fHBP B of said composition.

The composition may further comprise at least one of an fHBP A, NadA protein or dOMV.

According to another of its objects, the present disclosure relates to a method for preparing an immunogenic composition comprising a N. meningitidis fHBP A antigen, said composition inducing an enhanced immune response against a N. meningitidis serogroup B strain expressing an fHBP A heterologous to said fHBP A antigen of said composition, the method comprising at least the steps of:
a) selecting an AlPO₄ adjuvant having a PZC below 5, and
b) combining the AlPO₄ adjuvant selected at step a) with said fHBP A, and
c) obtaining said immunogenic composition.

The method allows obtaining an immunogenic composition able to induce an enhanced an immune response against a N. meningitidis serogroup B strain expressing a fHBP A heterologous to said fHBP A of said composition.

According to another of its objects, the present disclosure relates to a method for preparing an immunogenic composition comprising a N. meningitidis fHBP A antigen, said composition inducing an enhanced immune response against a N. meningitidis serogroup B strain expressing a fHBP A homologous to said fHBP A antigen of said composition, the method comprising at least the steps of:
a) selecting an AlPO₄ adjuvant having a PZC below 5, and
b) combining the AlPO₄ adjuvant selected at step a) with said fHBP A, and
c) obtaining said immunogenic composition.

The method allows obtaining an immunogenic composition able to induce an enhanced an immune response against a N. meningitidis serogroup B strain expressing a fHBP A homologous to said fHBP A of said composition.

The composition may further comprise at least one of a fHBP B, NadA protein or dOMV.

The methods of the disclosure may further comprise adding at least one antigen selected from fHBP A, NadA protein and dOMV. The addition of AlPO₄ adjuvant, fHBP B, fHBP A, NadA protein and dOMV may be carried out in any order.

A method of manufacturing of an immunogenic composition of the disclosure may comprise at least the steps of:
a) selecting an AlPO₄ adjuvant having a PZC below 5,
b) combining the AlPO₄ adjuvant selected at step a) with at least one factor H binding protein (fHBP) A, at least one factor H binding protein (fHBP) B, at least one NadA protein, and at least one dOMV, the combination being carried out in any order.

The fHBP A and B, the NadA protein and the dOMV may be as above detailed.

The fHBP A and B, the NadA protein and the dOMV may be sterile filtered before being combined together and with the AlPO₄ adjuvant.

Also, the methods of the disclosure may comprise a step of adding a further antigen. A further antigen may be a combination of MenACWY polysaccharides conjugated to a protein carrier as above detailed.

The disclosure relates to a method for stabilizing at least one of fHBP A and Neisseria adhesin A (NadA) protein in an immunogenic composition, the method comprising at least the steps of:
a) selecting an AlPO₄ adjuvant having a PZC below 5, and
b) combining the AlPO₄ adjuvant selected at step a) with fHBP A or NadA protein, and
c) obtaining an immunogenic composition in which said fHBP A or NadA protein is stabilized.

The method allows obtaining an immunogenic composition in which fHBP A and/or NadA is/are stabilized.

The methods of the disclosure may further comprise a step of adding at least one antigen.

For a composition comprising a NadA protein, the at least one further antigen may be selected from fHBP A, fHBP B and dOMV.

For a composition comprising a fHBP A, the at least one further antigen may be selected from fHBP B, NadA protein and dOMV.

The at least one further antigen may be added before or after the step of combining AlPO₄ with fHBP A or NadA protein.

The further antigens may each be added in separate steps to fHBP A or NadA protein or may be combined before being added in a single step. In the latter case they may be added as sub-combinations of antigens.

The step(s) of adding further antigens may be carried before or after combining AlPO₄ with fHBP A or NadA protein.

In some embodiments, step b) may comprise combining the AlPO₄ adjuvant to a combination of fHBP A, fHBP B, NadA protein and dOMV, the combination being carried out in any order.

The fHBP A and B, the NadA protein and the dOMV may be as above detailed.

Also, the method may comprise a step of adding a combination of MenACWY polysaccharides conjugated to a protein carrier as above detailed.

The stability of an antigen in a composition of the disclosure may be assessed by methods well-known in the art, including measurement of a sample's light scattering, apparent attenuation of light (absorbance, or optical density), size (e.g., by size exclusion chromatography), in vitro or in vivo biological activity and/or properties by differential scanning calorimetry (DSC).

For example, the stability of an antigen, such as NadA or fHBP A, in a composition of the disclosure may be determined by measuring the antigenicity of the considered antigen under thermal stress by incubating the immunogenic composition at 45° C. as a function of time, for example 0, 7, 14 and 28 days.

An antigen in a composition of the disclosure may maintain at least 50%, 60%, 70%, 75%, 80%, 85%, 90%, 95%, 96%, 97%, 98%, 99% or 100% of its antigenicity relative to a reference standard, e.g., the antigenicity measured at T₀ (i.e., the date of formulation or the date of a change in storage conditions), for at least 1 month, 2 months, 3 months, 4 months, 5 months, 6 months, 9 months, 12 months, 18 months, 24 months, 30 months, 36 months, 42 months, 48 months, at a temperature ranging from 4° C. to 8° C.

The combination of an AlPO₄ adjuvant of the disclosure with fHBP A and B is carried in conditions suitable for obtaining adsorption of fHBP A and B on AlPO₄.

The % of fHBP A and B adsorbed is as above indicated.

The fHBP A and B may each be adsorbed onto AlPO₄ in an amount of less than 85% of the total amount of fHBP B present in the composition, or in an amount ranging from about 50% to about 85%, or less, of the total amount of, respectively, fHBP A or fHBP B present in the composition.

Suitable conditions for ensuring adsorption of fHBP A and B on AlPO₄ include pH, temperature, and PZC of AlPO₄. Those conditions may be as above indicated.

For example, the pH may range from 5.5 to 7.0.

The temperature may range from 4° C. to 25° C.

A composition of the disclosure may be stored at a temperature ranging from about 4° C. to about 25° C. For example, a composition of the disclosure may be stored at a temperature of about 4° C. or at about 8° C., or else at about 4° C.

The present disclosure relates to a method for preparing an immunogenic composition comprising at least one N. meningitidis serogroup B antigen and having a time of onset of sedimentation (Tₒₙₛₑₜ) ranging from about 3.5 min to about 10 min, the method comprising at least the steps of:
a) selecting an AlPO₄ adjuvant having a PZC below 5, and
b) combining the AlPO₄ adjuvant selected at step a) with said at least one N. meningitidis serogroup B antigen, and
c) obtaining said immunogenic composition.

The at least one antigen may be fHBP A, fHBP B, NadA protein, or dOMV, and combination thereof.

The time of onset of sedimentation (Tₒₙₛₑₜ) may range from about 4 min to about 9 min, or from about 4.5 min to about 8.5 min.

Advantageously, a time of onset sedimentation of at least or above 3.5 min ensures that the components of the composition will remain in suspension during the manufacturing process, and therefore permitting more consistent manufacturing.

Further antigen, such as MenACWY polysaccharides conjugated to a protein carrier may be mixed with a composition as disclosed herein. The further antigen may be added to an immunogenic composition of the disclosure, just prior to administration to a patient, optionally via a dual chamber syringe that mixes a composition of the disclosure with at least a further antigen prior to the administration.

The manufacturing methods of the disclosure may be used to manufacture a vaccine.

### Uses and Methods

The present disclosure relates to an immunogenic composition as disclosed herein for use as a medicament, in particular as a vaccine.

An immunogenic composition of the disclosure or a vaccine comprising the immunogenic composition of the disclosure may be for use in a method for protecting against a meningococcal infection. The meningococcal infection may be caused by a N. meningitidis serogroup B strain.

An immunogenic composition or a vaccine of the disclosure may be for use in a method for inducing an immune response against a N. meningitidis serogroup B strain.

An immunogenic composition or a vaccine of the disclosure may be for use in a method for protecting an individual against a meningococcal infection, the method comprising at least a step of administering to the individual said immunogenic composition.

The disclosure relates to a method for protecting an individual against a meningococcal infection, the method comprising at least a step of administering to the individual said immunogenic composition or a vaccine comprising said immunogenic composition.

An immunogenic composition or a vaccine of the disclosure may be for use in a method for reducing a risk of occurrence of an invasive meningococcal disease caused by a meningococcal infection in an individua, the method comprising at least the step of administering to the individual said immunogenic composition or said vaccine.

The disclosure relates to a method for reducing a risk of occurrence of an invasive meningococcal disease caused by a meningococcal infection in an individual, the method comprising at least the step of administering to the individual an immunogenic composition or a vaccine of the disclosure.

An immunogenic composition or a vaccine of the disclosure may be for use in a method for eliciting an immune response against a N. meningitidis serogroup B strain in an individual, the method comprising at least the step of administering to the individual said immunogenic composition or said vaccine.

The disclosure relates to a method for eliciting an immune response against a N. meningitidis serogroup B strain in an individual, the method comprising at least the step of administering to the individual an immunogenic composition or a vaccine of the disclosure.

The meningococcal infection may be a N. meningitidis serogroup B infection. The immunogenic composition may be a vaccine.

The disclosure relates to a use of an AlPO₄ adjuvant having a PZC below 5 for enhancing an immune response induced by a composition comprising a N. meningitidis fH-BP B antigen against a N. meningitidis serogroup B strain expressing an fHBP B heterologous to said fHBP B antigen of said composition.

The disclosure relates to an AlPO₄ adjuvant having a PZC below 5 in an immunogenic composition comprising a N. meningitidis fH-BP B antigen for use in a method for enhancing an immune response induced by said composition against a N. meningitidis serogroup B strain expressing a fHBP B heterologous to said fHBP B antigen of said composition.

According to another of its objects, the present disclosure relates to a use of an AlPO₄ adjuvant having a PZC below 5 for enhancing an immune response induced by a composition comprising a N. meningitidis fH-BP B antigen against a N. meningitidis serogroup B strain expressing a fHBP B homologous to said fHBP B antigen of said composition.

According to another of its objects, the present disclosure relates to a use of an AlPO₄ adjuvant having a PZC below 5 for enhancing an immune response induced by a composition comprising a N. meningitidis fHBP A antigen against a N. meningitidis serogroup B strain expressing a fHBP A heterologous to said fHBP A antigen of said composition.

According to another of its objects, the present disclosure relates to a use of an AlPO₄ adjuvant having a PZC below 5 for enhancing an immune response induced by a composition comprising a N. meningitidis fHBP A antigen against a N. meningitidis serogroup B strain expressing a fHBP A homologous to said fHBP A antigen of said composition.

According to another of its objects, the present disclosure relates to a use of an AlPO₄ adjuvant having a PZC below 5 for stabilizing at least one fHBP A in an immunogenic composition.

The disclosure relates to a use of an AlPO₄ adjuvant having a PZC below 5 for stabilizing at least one Neisseria adhesin A (NadA) antigen in an immunogenic composition.

The disclosure relates to a use of an AlPO₄ adjuvant having a PZC below 5 for stabilizing a time of onset of sedimentation (Tₒₙₛₑₜ) of a composition comprising at least one Neisseria meningitidis serogroup B antigen, in a range from about 3.5 min to about 10 min, or in a range from about 4 min to about 9 min, or in a range from about 4.5 to about 8.5 min.

The at least one Neisseria meningitidis serogroup B antigen may be from a group comprising fHBP A, fHBP B, NadA protein, dOMV, and combinations thereof.

The at least one Neisseria meningitidis serogroup B antigen may be a combination of fHBP A and fHBP B.

The disclosure relates to a use of an AlPO₄ adjuvant having a PZC below 5 for adjuvanting an immunogenic composition comprising a combination of Neisseria meningitidis serogroup B antigens, said combination comprising at least one factor H binding protein (fHBP) A and at least one factor H binding protein (fHBP) B.

The disclosure relates to a use of an AlPO₄ adjuvant having a PZC below 5 for manufacturing an immunogenic composition comprising a combination of Neisseria meningitidis serogroup B antigens, said combination comprising at least one factor H binding protein (fHBP) A and at least one factor H binding protein (fHBP) B.

The disclosure relates to a method for inducing an immune response against a Neisseria meningitidis serogroup B strain in an individual in need thereof, the method comprising at least a step of administering to said individual an immunogenic composition according to the disclosure, wherein said step of administration induces an immune response against said Neisseria meningitidis serogroup B strain.

The disclosure relates to a method for enhancing an immune response induced by a composition comprising a N. meningitidis fHBP B antigen against a N. meningitidis serogroup B strain expressing a fHBP antigen heterologous to said fHBP B antigen of said composition, in an individual in need thereof, the method comprising at least a step of administering to said individual an immunogenic composition according to the disclosure, wherein said step of administration induces an enhanced immune response against said heterologous Neisseria meningitidis serogroup B strain.

Individuals concerned by methods and uses of the disclosure may be mammals, for example human beings, and for example infants, toddlers, children, teenagers, young adults, adults, and seniors. In one embodiment, an individual may be from 6 weeks-old or more, 2 months-old or more, or 10 years-old or more. As exemplary embodiments, an individual may be from 6-weeks to 55 years-old or more, for example from 2 months to 55 years-old or more, or for example from 10 to 55 years-old or more.

The methods generally involve administering to an individual in need thereof an effective amount of a subject immunogenic composition. Amounts effective for therapeutic use will depend on, e.g., the antigenic composition, the manner of administration, the weight and general state of health of the patient, and the judgment of the prescribing physician. Single or multiple doses of the antigenic compositions may be administered depending on the dosage and frequency required and tolerated by the patient, and route of administration.

Immunogenic compositions as disclosed here may be administered in a 2, 3, 2+1, or 3+1 doses regimen.

In one embodiment, an immunogenic composition disclosed herein may be administered in 2 or 3 doses. The subsequent dose may be administered about one, about two, about three, about four, about five, about six, about seven, about eight, about nine, about ten, about eleven, about twelve, about thirteen, about fourteen, about fifteen, about sixteen, about seventeen, about eighteen, about nineteen or about twenty months apart from the previous one. In one embodiment, the subsequent dose may be administered about one, about two, about five, about six, about eight, about ten, about twelve, about fourteen or about sixteen months apart from the previous one. In one embodiment, the subsequent dose may be administered about one, about two, about five, about six, or about eight, months apart from the previous one. In one embodiment, the subsequent dose may be administered about 30 days, about 60 days, or about 180 days apart from the previous one.

In a two doses regimen, the second dose may be administered about one month after the first dose, or about 2 months after the first dose, or after 6 months after the first dose. Alternatively, in a two doses regimen, the second dose may be administered about 30 days after the first dose, or about 60 days after the first dose or about 180 days after the first dose. Such two-doses regimen may be suitable for adults and/or adolescents.

In a two doses regimen, the second dose may be administered about 2 months after the first dose. Alternatively, in a two doses regimen, the second dose may be administered about 60 days after the first dose. Such two-doses regimen may be suitable for toddlers.

In a three doses regimen, the second dose may be administered about one month after the first dose and the third dose may be administered about 6 months after the first dose. Alternatively, in a three doses regimen, the second dose may be administered about 30 days after the first dose and the third dose may be administered about 180 days after the first dose. Such three-doses regimen may be suitable for adults and/or adolescents.

In a three doses regimen, the second dose may be administered about two months after the first dose and the third dose may be administered about 10 months after the first dose. Alternatively, in a three doses regimen, the second dose may be administered about 60 days after the first dose and the third dose may be administered at about 12 months of age. Such three-doses regimen may be suitable for infants.

In one embodiment, further to the 2 or 3 doses, a third or fourth dose may be administered. This subsequent dose may be administered at least one year after the last dose of the 2 or 3 doses, for example 16 months after the last dose. In such regimen the first two or three doses may be qualified as prime doses, and the subsequent one (+1) may be qualified as a boost dose.

In one embodiment, infants and toddlers, for example from 6-weeks or 2-months to 2 years-old may receive a 2+1 or a 3+1 doses regiment. In another embodiment, children, for example from 2 to 10 years-old, may receive a 2 doses regimen. In another embodiment, teenagers and adults, for example from 10 to 55 years-old may receive a 2+1 doses regimen.

Immunogenic compositions as disclosed herein may be administered by any suitable route. For example, administration by intramuscular route may be considered.

### EXAMPLES

The following examples illustrate the embodiments of the disclosure that are presently best known. However, it is to be understood that the following are only exemplary or illustrative of the application of the principles of the present disclosure. Numerous modifications and alternative compositions, methods, and systems may be devised by those skilled in the art without departing from the spirit and scope of the present disclosure.

### Example 1: Materials & Methods

Formulation of AlPO₄ with Low Point Zero Charge (Mod-AlPO₄ Adjuvant)

Aluminum phosphate adjuvant gel with a PZC ranging between 5 to 7 was titrated with a phosphate buffer/salt solution that allows for the exchange of phosphate groups on the buffer or salt solution with the hydroxyl groups on the surface of aluminum phosphate adjuvant. The PZC of the AlPO₄ can be lowered by using any orthophosphate or phosphate donor salt or buffer solutions.

Different methods can be used to obtain AlPO₄ with a target PZC.

In one method, phosphate buffer pH 5.8 formulated by a combination of 0.5 M sodium phosphate monobasic and 0.5 M sodium phosphate dibasic was added to make AlPO₄ with a different PZC.

In a second method, the PZC of AlPO₄ was modified by titrating the AlPO₄ with a 0.5 M stock solution of sodium phosphate monobasic salt.

AlPO₄ adjuvant with PZC below 5 is a modified AlPO₄ adjuvant and is hereafter, in the Examples section, referred to as “mod-AlPO₄ adjuvant”. To prepare 100 mL of modified AlPO₄, adjuvant (mod-AlPO₄ adjuvant), 80 mL of AlPO₄ (concentration 4.8 mg Al/mL) was combined with 20 mL of 0.5 M sodium phosphate buffer pH 5.8 at room temperature and stirred for no less than 30 minutes. The modified AlPO₄ (mod.AlPO₄ or mod-AlPO₄) was stored at 2-8° C. until used.

AlPO₄ adjuvant with PZC above 5 is a non-modified AlPO₄ adjuvant and is hereafter, in the Examples section, named “AlPO₄ adjuvant”.

In the following of the studies, mod-AlPO₄ adjuvant is modified as above described to set a PZC at about 4.5, while AlPO₄ adjuvant is set to have a PZC at about 5.2.

### Preparation of MenB Immunogenic Composition (MenB)

Non-Lipidated Mutated fHBP A05 (A05tmN)

For preparing the non-lipidated A05tmN, three point-mutations (G220S, L130R and G133D numbering with respect to SEQ ID NO: 6) were introduced into the wild-type fHBP A05 sequence. Additionally, the lipidatable cysteine residue at the N-terminus was replaced by a methionine residue (non-lipidated A05tmN: SEQ ID NO: 8). The DNA sequence encoding the A05tmN antigen was synthesized and then cloned into a plasmid construct. In short, the DNA sequences of Xba1 and Xho 1 sites were added on both ends of the A05tmN sequence. To create the expression plasmids, Xba1/Xho1-containing pET28a(+) plasmids were digested. The DNA sequences encoding the A05tmN with Xba1 and Xho 1 sites were ligated into the Xba1/Xho1 digested pET28a(+) and transformed into Top10 competent cells. A positive clone was identified and confirmed by Xba 1/Xho 1 digestion The A05tmN plasmid was transformed into E. coli, and a cell bank was manufactured after three rounds of colony purification.

The E. coli strain transformed with the A05tmN expression construct and was amplified in semi-defined medium at 37° C. under agitation (pH 6.8—Dissolved oxygen: 20%). Expression of the antigen was induced by addition of isopropyl β-D-1-thiogalactopyranoside (IPTG).

The culture was harvested as unprocessed bulk and the bacterial biomass was separated from the media with centrifugation. The resulting cell pellet was resuspended in a buffer (20 mM Tris-HCl, pH 8.5). The resuspended pellet was processed through a homogenizer to produce cell homogenate. The homogenate was subsequently centrifuged to collect the pellet fraction. The homogenate pellet was resuspended in buffer (20 mM Tris-HCl, pH 8.5) and subjected to a pH shock treatment (pH 12 for 1 hour at room temperature with mixing). The pH was brought back down to 8.5 with 85% phosphoric acid. The supernatant fraction of the pH-shocked material was collected following centrifugation and then filtered to obtain a filtered supernatant.

The supernatant was conditioned to pH 8.5 and <5.0 mS/cm conductivity and loaded onto a capture column, GigaCap Q-650M. The elution pool is conditioned to 0.9 M ammonium sulfate (AmS), then further purified with the intermediate chromatography, Toyopearl Phenyl 600M. After the hydrophobic interaction chromatography, the eluted pool is conditioned to pH 8.5 and <8.0 mS/cm conductivity, and further purified through Nuvia aPrime 4A chromatography. This is followed by the final ultrafiltration and diafiltration using 5 kDa regenerated cellulose tangential flow filtration (TFF) membrane, and a 0.2-μm filtration.

Non-Lipidated Mutated fHBP B01 (B01smN)

For preparing the non-lipidated B01smN, a single point-mutation (H248L numbering with respect to SEQ ID NO: 6) was introduced into the wild-type fHBP B01 sequence. Additionally, the lipidatable cysteine residue at the N-terminus was replaced by a methionine (non-lipidated B01smN: SEQ ID NO: 9). The DNA sequence for B01smN was synthesized and then cloned into a plasmid construct. In short, the DNA sequences of Xba1 and Xho 1 sites were added on both ends of the B01smN sequence. To create the expression plasmids, Xba1/Xho1-containing pET28a(+) plasmids were digested. The DNA sequences encoding the B01smN with Xba1 and Xho 1 sites were ligated into Xba1/Xho1 digested pET28a(+) and transformed into Top10 competent cells. A positive clone was confirmed by Xba 1/Xho 1 digestion. The B01smN plasmid was transformed into E. coli and the cell bank was manufactured after three rounds of colony purification.

The E. coli strain transformed with the B01smN expression construct and was amplified in semi-defined medium at 37° C. under agitation (pH 6.8—Dissolved oxygen: 20%). Expression of the antigen was induced by addition of isopropyl β-D-1-thiogalactopyranoside (IPTG).

The culture was harvested as unprocessed bulk and the bacterial biomass was separated from the media with centrifugation. The resulting cell pellet was resuspended in a buffer (20 mM Tris-HCl, pH 8.5). The resuspended pellet was processed through a homogenizer to produce cell homogenate. The homogenate was subsequently centrifuged to collect the supernatant fraction. The supernatant faction was then filtered.

The filtered supernatant was conditioned to pH 8.5 and <5.0 mS/cm conductivity and loaded onto a chromatography CaptoQ ImpRes and purified in a bind and elute mode. The CaptoQ ImpRes elution pool is then conditioned to 1.8 M AmS for loading onto the second chromatography, Phenyl Sepharose HP. After elution, the material was concentrated and diafiltered into the acetate buffer (50 mM sodium acetate, 150 mM NaCl, pH 6.0) using 5 kDa Ultracel TFF membrane, then 0.2-μm filtered.

### NadA

A truncated version of NadA was prepared from NadA_MC58. The truncated NadA lacks the leader sequence (residues 1 to 23) and anchor domain (residues 308 to 362) of NadA_MC58 (truncated NadA: SEQ ID NO: 5). In the truncated sequence of NadA_MC58, the first amino acid after the leader sequence is alanine, which is replaced by a methionine. The DNA sequence encoding truncated NadA was synthesized and then cloned into a plasmid construct. DNA sequences of Xba1 and Xho1 sites were added on both ends of the NadA sequence. To create the expression plasmids, Xba1/Xho1-containing pET28a(+) plasmids were digested. The DNA sequences encoding the NadA with Xba1 and Xho 1 sites were ligated into Xba1/Xho1 digested pET28a(+) and transformed into Top10 competent cells. A positive clone was confirmed by Xba1/Xho1 digestion. The NadA plasmid was transformed into E. coli and the cell bank was manufactured after three rounds of colony purification.

The E. coli strain transformed with NadA1 was amplified in semi-defined medium at 37° C. under agitation (pH 6.8—Dissolved oxygen: 20%). Expression of the antigen was induced by addition of isopropyl β-D-1-thiogalactopyranoside (IPTG).

The culture was harvested as unprocessed bulk and the bacterial biomass was separated from the media with centrifugation. The resulting cell pellet was resuspended in a buffer (20 mM Tris-HCl, pH 8.5). The resuspended pellet was processed through a homogenizer to produce cell homogenate. The homogenate was subsequently centrifuged to collect the supernatant fraction. The supernatant faction was then filtered.

The supernatant fraction was loaded onto a Capto DEAE column. The Capto DEAE elution fraction is conditioned with powdered AmS until a concentration of 500 mM AmS is achieved. The conditioned Capto DEAE elution fraction is loaded onto a Toyopearl Butyl-650M column. The Toyopearl Butyl-650M elution fraction was loaded onto a CHT Type I 40 μm column and the CHT elution fraction is concentrated using a 30 kDa regenerated cellulose TFF membrane followed by diafiltration into 50 mM sodium acetate, 150 mM NaCl, pH 6.0. Following TFF, the product is 0.2-μm filtered to produce the NadA antigen.

dOMV

The dOMV was purified from wild-type N. meningitidis (Nm) serotype B strain 99M that was provided by the Walter Reed Army Institute of Research (WRAIR).

The Nm B 99M was cultured in a chemically defined medium described in Fu et al. (Biotechnology (N Y). 1995 February; 13(2):170-4) and in U.S. Pat. No. 5,494,808 in presence of yeast extract at 1 g/L and Hepes 1 M, at 37° C., under CO₂ 5%.

Culture harvest was carried out using a low-speed centrifugation of the heat-treated suspension (55° C. for 2 hours) in order to recover wet bacterial pellets. Two serial detergent-mediated extraction steps (56° C. for 15 minutes) were performed with extraction buffer composed of a detergent (sodium deoxycholate) to extract dOMV from the bacterial outer membrane and deplete lipooligosaccharides (as disclosed in Helting et al., Acta Pathol Microbiol Scand C. 1981 April; 89(2):69-78). The sodium deoxycholate and EDTA solubilize the bacterial outer membranes, which then re-organize themselves into dOMV (vesicles and particulates). Resuspension was completed by using Ultra-Turrax (rotor-stator equipment) to homogenize the pellets suspended in the extraction buffer. The dOMV supernatants were pooled before benzonase treatment in the presence of MgCl₂ (37° C. for 15 minutes).

Following the dOMV extraction, the dOMV was concentrated using hollow fibers in modified PolyEtherSulfone (mPES) of 300 kDa. Several ultracentrifugation steps were used to separate dOMV from the “soluble” content such as nucleic acids, cytosolic proteins, extracted lipopolysaccharides or buffer components. The resulting pellets were then resuspended in the extraction buffer using Ultra-Turrax (rotor-stator equipment) at the minimum speed for a few seconds. After primary resuspension, high pressure homogenization was used to fully resuspend dOMV in the extraction buffer and to increase accessibility of detergent to the dOMV surface.

Centrifugation was then performed prior to final filtration of supernatant with a 0.45/0.2-μm cellulose acetate filter. The dOMV were harvested in water for injection (WFI).

### MenB Immunogenic Compositions

The MenB antigens in the MenB immunogenic composition are purified non-lipidated mutated A05 fHBP (A05tmN), non-lipidated mutated fHBP (B01smN), NadA, and dOMV. The A05tmN, B01smN, NadA and dOMV antigens were combined to aluminum phosphate adjuvant (AlPO₄) (PZC 5.2) or to mod-AlPO₄ adjuvant (PZC 4.5).

The vehicle was consisting of acetate buffer (50 mM sodium acetate, 150 mM NaCl, pH 6.0).

The AlPO₄ adjuvant was either non-modified AlPO₄ adjuvant (PZC 5.2; 1.00 mg Al/mL) or mod-AlPO₄ adjuvant (PZC 4.5; 1.00 mg Al/mL) prepared as above indicated.

To formulate the immunogenic compositions formulations, non-modified (PZC 5.2) or mod-AlPO₄ (PZC 4.5) adjuvant, B01smN, A05tmN, NadA protein, dOMV, and acetate buffer (50 mM sodium acetate, 150 mM NaCl, pH 6.0) were blended together to achieve target antigen and aluminum concentrations (100 μg/mL for B01smN, 100 μg/mL for A05tmN, 100 μg/mL for NadA, 250 μg/mL for dOMV, and 1.00 mg Al/mL of AlPO₄).

Final aluminum concentration at 0.8 mg Al/mL of AlPO₄ was retained for stability data and PZC comparison (4.5 and 4.8). The adsorbed antigen is measured by determining the non-adsorbed amounts of antigen using RP-HPLC, for the MenB antigens, and HPAEC-PAD for the ACYW conjugates.

The estimated amount of residual phosphate buffer in the final composition is about 24 mM.

### Preparation of MenACWY and MenPenta Immunogenic Compositions

### Preparation of the MenACWY Immunogenic Composition

The MenACWY immunogenic composition is obtained from MENQUADFI®. MENQUADFI© is a commercially available vaccine comprising ACWY polysaccharides antigens obtained and conjugated to tetanus toxoid (TT) as disclosed in WO 2018/045286 A1. The formulation comprises the N. meningitidis capsular polysaccharides from serogroups A, C, Y, and W135, separately conjugated to tetanus toxoid protein. The target active ingredients concentrations are 10 μg of each polysaccharide and approximately 55 μg of tetanus toxoid protein per 0.5 mL dose. The antigens were formulated in a sterile, aqueous solution containing 30 mM sodium acetate buffer (1.23 mg/dose) and sodium chloride (0.67%, 3.35 mg/dose).

### Preparation of the MenPenta Immunogenic Composition (MenPenta)

The MenPenta formulation was prepared by combining the two non-lipidated factor H binding proteins (fHBP) from subfamily A and B, the Neisseria adhesin A (NadA), and detergent-extracted outer membrane protein vesicles (dOMV) targeting N. meningitis B strains, prepared as above indicated, and serogroups polysaccharides A, C, Y and W135 conjugated to tetanus toxoid as carrier, obtained as above indicated, with (i) aluminum phosphate adjuvant (AlPO₄) (PZC 5.2) or (ii) mod-AlPO₄ adjuvant (PZC 4.5).

The preparation was mixed by stirring at ambient temperature for no less than 30 minutes and stored at 2-8 C until used.

### Time of Onset of Sedimentation

Sedimentation studies were done using the TURBISCAN LAB™ in which 1.5 mL volume of the composition to be assayed was filled into a 4-mL glass tube and loaded in an adapter that matches the vial size. Samples of the MenB or MenPenta compositions with AlPO₄ at PZC 5.2 or 4.5 (obtained as indicated above) were monitored every 25 to 30 seconds for up to 30 minutes for transmission and back scattering. Both transmission and back scattering were measured. Measurement was performed scanning from the bottom to top of the measurement cell. The temperature for measurement was set at 28° C.

### Pyrogenicity of Immunogenic Compositions (Measure of IL-6 EC₅₀)

Pyrogenicity of the tested immunogenic compositions (MenB with AlPO₄ PZC 5.2 or with mod-AlPO₄ PZC 4.5—prepared as above-indicated) was assayed using the Monocyte Activation Test (MAT).

The MAT is based on the capacity of human monocyte cells to secrete endogenous pyrogens (proinflammatory cytokines) in response to the detection of exogenous pyrogens contained in the test sample. Monocyte Activation Test (MAT) works by predicting the human response to pyrogens on the same endogenous cytokines that are produced during a human fever. For test samples with inherent pyrogens (e.g., lipoproteins, lipooligosaccharides, other unknown components), MAT allows to quantify intrinsic pyrogenicity and is used to document consistency.

MAT is based on a pool of peripheral blood mononuclear cells (PBMCs) provided from eight different healthy human donors as the monocyte cell source and human Interleukin-6 (IL-6) as the read-out.

In the present study, the goal was to compare the pyrogenicity of immunogenic compositions prepared with either with AlPO₄ adjuvant (PZC 5.2) or with mod-AlPO₄ adjuvant (PZC 4.5). Samples were all adjusted to the same protein content, serially diluted, plated in 96 microwell plates with human PBMCs, and incubated overnight. After incubation, supernatant was transferred into a 96 microplate and immune detection of IL-6 was performed by homogeneous time resolved fluorescence (HTRF). The half-maximal effective concentration (EC₅₀) as calculated on the 4PL extrapolation model of the dose response curve is the parameter used to compare the intrinsic pyrogenicity of each composition. The smallest is the EC₅₀ and the highest is the intrinsic pyrogenicity of the tested product.

### Measure of Antigens Adsorption on Aluminum

The percentage of adsorption of antigens to the mod-AlPO₄ or AlPO₄ was measured by determining the concentration of non-adsorbed antigens obtained in supernatants after centrifugation of the aluminum adjuvant-bound antigens relative to the total concentration using the formula:

$$( 1 - \frac{Concentration of antigen in supernatant}{Total concentration of the antigen in test sample} ) \times 100 \%$$

Concentrations of A05tmN, B01smN, NadA and dOMV were determined by reverse phase high performance liquid chromatography (RP-HPLC).

The Reverse Phase Liquid Chromatography (RP-LC) method from Nompari et al. Talanta 178 (2018) 552-562) was implemented on an Agilent 1260 Infinity HPLC instrument with UV detection.

To determine the % adsorption, antigen content of the supernatant and desorbed (bound) fractions were evaluated.

The supernatant sample was prepared by centrifuging the adsorbed sample at 3000 rcf at 22° C. for 5 mins. An appropriate volume of the supernatant was mixed with the detergent Zwittergent 3-14 to obtain a final concentration of 0.1% w/v Zwittergent 3-14 in the sample with minimum sample dilution (˜0.95×). The samples were then heated in a microtube heater at 60° C. for 1 hour with shaking at 300 rpm. To prepare the total sample, desorption of antigens was achieved by treating the entire adsorbed sample (total) with 5% w/v citrate, an aluminum chelation agent, and 0.1% Zwittergent 3-14. All samples and standards were heated at 60° C. for 1 hour with shaking at 300 rpm, and centrifuged at 3000 rcf for 22° C. for 5 min prior to storage in the HPLC autosampler at 10° C. prior to HPLC analysis.

The Reference Standard working solutions were prepared fresh on the day of testing by diluting the antigen reference standards of A05tmN, B01smN and NadA to appropriate concentration in 150 mM sodium acetate plus 50 mM sodium chloride pH 6.3 buffer with 0.1% Zwittergent 3-14. The dOMV protein components were quantified using NadA as a heterologous reference standard.

Optimal separation of all antigens was achieved using the BioResolve RP mAb Polyphenyl Column, 450 Å, 2.7 μm, 2.1 mm×150 mm from Waters. Liquid chromatography mass spectrometry (LC-MS) grade 0.1% TFA in water and 0.1% TFA in ACN were used as the aqueous and organic mobile phases, respectively. The chromatographic gradient started with 10% organic phase to a final of 80% organic phase at a column temperature of 70° C. The detection wavelength used was 215 nm. The protein concentration was determined by interpolating the amount of each antigen in nanograms (ng) from the appropriate calibration curve then dividing it by the injection volume (in μL) to yield ng/μL or μg/mL.

The concentrations of the tetanus toxoid protein-conjugated N. meningitidis capsular polysaccharides serogroups A, C, Y, and W135 were determined by high-performance anion-exchange chromatography/pulsed amperometric detection (HPAEC-PAD).

Quantitation of polysaccharide content within drug product vaccine and % adsorption to AlPO₄ was evaluated through sample preparation, acid hydrolysis to yield monosaccharides, and individual monosaccharide analysis using chromatography performed by HPAEC-PAD.

Desorbed samples were prepared by centrifuging an aliquot of the drug product sample at 14000 rpm at RT for 30 min. Supernatant were removed and diluted within standard curve range using sodium acetate/sodium chloride buffer. An aliquot of the drug product sample (adsorbed) was also diluted into the standard curve range using sodium acetate/sodium chloride buffer. The reference standards for the diluted and adsorbed drug product, supernatant, and polysaccharide were prepared in sample set.

Hydrolysis and chromatography conditions were adapted from those described in Gudlavalleti et al (Anal Chem. 2014 Jun. 3; 86(11):5383-90. doi: 10.1021/ac5003933. Epub 2014 May 20. PMID: 24810004.).

Calibration curves were calculated for each serogroup A, C, W, Y from a tetravalent polysaccharide-based reference standard using linear regression. Polysaccharide concentration of both adsorbed and desorbed (supernatant) samples of A, C, W, Y were interpolated from their respective curves. The % adsorption was calculated as desorbed/drug product sample×100.

Evaluation of the Immunogenicity of MenB Immunogenic Compositions Formulated with Mod-AlPO₄ Adjuvant (PZC 4.5) or with AlPO₄ Adjuvant (PZC 5.2)

### Products Under Test

TABLE 1
PRODUCTS UNDER TEST

| Formulation | Concentration |
|---|---|
| MenB without AlPO₄ - | 100 μg/mL of fHBP A05 tmN |
| Group A | 100 μg/mL of fHBP B01smN |
| | 100 μg/mL of NadA |
| | 250 μg/mL of dOMV |
| MenB + AlPO₄ adjuvant | 100 μg/mL of fHBP A05 tmN |
| (PZC 5.2) - Group B | 100 μg/mL of fHBP B01smN |
| | 100 μg/mL of NadA |
| | 250 μg/mL of dOMV |
| | 0.8 mg/ml of AlPO₄ adjuvant |
| MenB + mod-AlPO₄ adjuvant | 100 μg/mL of fHBP A05 tmN |
| (PZC 4.5) - Group C | 100 μg/mL of fHBP B01smN |
| | 100 μg/mL of NadA |
| | 250 μg/mL of dOMV |
| | 0.8 mg/ml of mod-AlPO₄ adjuvant |

### Dosing-Regimen

Three groups (A, B and C) of eight rabbits each (female; strain: NZW KBL; 9-10 weeks old on DO) were used. Group A received the MenB immunogenic composition without AlPO₄, while groups B and C received a MenB immunogenic composition formulated with 400 μg of either AlPO₄ adjuvant (PZC 5.2) or mod-AlPO₄ adjuvant (PZC 4.5), respectively. These formulations were administered via intramuscular (IM) injections on D0 and D28 (500 μL in the right thigh for the first injection and 500 μL in the left thigh for the second injection). Blood samples were collected under local anesthesia at the median artery of the rabbit's ear on D0, D28 and D42 (two weeks after the last injection) for all groups.

TABLE 2
DOSING REGIMEN

| | | | Route / | |
|---|---|---|---|---|
| | Antigens | | Volume | Animal / |
| Group | quantity /dose | Adjuvant | injected | group |
| A | 50 μg of fHBP A05 tmN | None | IM - | 8 |
| | 50 μg of fHBP B01smN | | 500 μL | |
| | 50 μg mL of NadA | | | |
| | 125 μg of dOMV | | | |
| B | 50 μg of fHBP A05 tmN | AlPO₄ | IM - | 8 |
| | 50 μg of fHBP B01smN | (PZC 5.2) | 500 μL | |
| | 50 μg mL of NadA | | | |
| | 125 μg of dOMV | | | |
| C | 50 μg of fHBP A05 tmN | mod-AlPO₄ | IM - | 8 |
| | 50 μg of fHBP B01smN | (PZC 4.5) | 500 μL | |
| | 50 μg mL of NadA | | | |
| | 125 μg of dOMV | | | |

Biological Sampling and hSBA

### Biological Sampling

On D0 and D42, blood samples were taken from the median artery of the ear under local anesthesia. Local anesthesia was performed by spreading anesthetic cream (Elma®) on the rabbit ear 5 min before the blood sample was taken. The rabbits were also maintained under chemical anesthesia with Rompun and Imalgene products.

Blood samples were collected in tubes containing clot activator and serum separator (BD Microtainer SST 5 mL, ref 15388989). The tubes were centrifugated at 3500 rpm for 15 min in order to separate serum from blood cells. The sera were transferred into Deepwell (ritter) plates and heat-inactivated at 56° C. for 30 min. They were stored at −20° C. until use for IgG purification and bacterial killing assays.

hSBA
IgG Purification of Rabbit Sera for hSBA Testing

To avoid non-specific bactericidal killing induced by rabbit sera collected on D0 and D42, a purification of IgG was necessary.

Purification of rabbit sera was performed using rProtein A GravtiTrap™ columns (GE healthcare GE28-9852-54) and Ab Buffer Kit GE Healthycare ref 28-9030-59).

First, the columns were equilibrated with binding buffer (phosphate sodium 20 mM pH=7). After adjustment of pH sera to 7 with binding buffer (V/V), the sera were added to the column to perform IgG binding. The column was washed with binding buffer. Elution buffer (Glycine HCl 0.1M pH2.7) was then added to the column to collect IgG. To preserve activity of IgG, neutralizing buffer (Tris-HCl 1M, pH9.0) was added to the elution fraction to obtain a final pH of approximately 7. Quantification of IgG concentration was done by Nanodrop.

### Serum Bactericidal Activity

The bactericidal titers of individual purified sera (purified IgG) from immunized rabbits were measured by in vitro quantification of antibody-dependent complement-mediated killing of Neisseria meningitidis serogroup B (or Serum Bactericidal Activity—SBA). This assay was performed in the presence of human complement (hSBA). In the presence of complement and some classes of immunoglobulins, a lytic complex antigen-antibody is obtained on the surface of the target bacteria causing its death. By observing a bactericidal effect resulting from the target candidate specific antibody present in sera, the SBA level of the sera can be determined.

The bactericidal titer is the dilution yielding ≥50%. The number of resulting bacterial colonies present in the wells is inversely proportional to the level of functional antibodies present in the serum, which is directly proportional to the immunological response of the animal or human subject.

The SBA assay measures the ability of antibodies to lyse bacteria in the presence of complement. The bactericidal titer of a serum is defined as the reciprocal of the highest dilution of the test serum that results in at least a 50% killing compared to the complement control wells containing no serum.

The number of resulting bacterial colonies present in the wells is inversely proportional to the level of functional antibodies present in the serum, which is directly proportional to the immunological response of the animal or human subject.

The source of complement was a human complement (Ig-depleted human serum). Briefly, sera were heat-inactivated 30 min at 56° C. and subsequently serially two-fold diluted (9 times) in a 96-well microplate either in Dulbecco PBS buffer containing Ca²⁺ and Mg²⁺ and 0.2% gelatin or in a Dulbecco PBS buffer containing Ca²⁺/Mg²⁺, 0.1% dextrose and 0.5% Bovine Serum Albumin.

All serum which are assessed in SBA assay are heat inactivated for 30 min in a 56° C. water bath to inactivate intrinsic complement activity.

Preculture of bacteria was performed onto Mueller Hinton agar (Petri dish) for 18 h at 37° C. in 5% CO₂ to obtain confluent bacterial growth.

Thereafter, bacteria were grown in Brain Heart Infusion (BHI) medium (supplemented with 4-HPA 5 mM for strain n° 6—see table 3—to induce NadA expression), for 2 h30 at +37° C. with shaking (100 rpm).

Meningococcal bacteria were diluted to obtain 1.4 10⁴ CFU/mL. 25 μL of working bacteria suspension, 50 μL of pre-diluted sera and 25 μL of diluted human complement (final concentration 15%) were deposited in a 96-well microplate and incubated at +37° C. for 1 hour with shaking (100 rpm). The Zephyr robotic application automatically deposited 40 μL of each well on square plate with Mueller Hinton agar (40*40). Agar plates were incubated at +37° C. with 5% CO₂ for 12±4 hours.

After incubation, the number of colonies per well was counted by using Cybele Software from Microvision company.

The bactericidal titer was defined as the dilution of the test serum that resulted in at least a 50% decrease in colony forming units (CFUs) per mL of bacteria compared to complement control well. Analyses were performed using Softmax Pro v6.5.1 GXP integrated in Sanofi Universal Exporter (SUE) and by selecting SBA WARP module.

Neisseria meningitidis Serogroup B Strains for SBA Evaluation

TABLE 3
NEISSERIA MENINGITIDIS SEROGROUP
B STRAINS FOR SBA EVALUATION

| Strain no | fHBP variant | PorA variant | NadA variant |
|---|---|---|---|
| 1 | B44 | 1.14 | Absent |
| 2 | A56 | 1.14 | Off frameshift |
| 3 | B24 | 1.16 | Absent |
| 4 | A22 | 1.1 | Absent |
| 5 | A10 | 1.2 | Absent |
| 6 | B79 | 1.15 | NadA1 |

### Data Analysis

An ANOVA analysis was performed with product as fixed factor.

Evaluation of the Immunogenicity of MenACWY or MenPenta Immunogenic Compositions Formulated with Mod-AlPO₄ Adjuvant (PZC 4.5)

### Products Under Test

TABLE 4
PRODUCTS UNDER TEST

| Formulation | Concentration |
|---|---|
| MenACWY | 20 μg/mL Neisseria meningitidis group A polysaccharide |
| without | conjugated to tetanus toxoid carrier protein |
| AlPO₄ - | 20 μg/mL Neisseria meningitidis group C polysaccharide |
| Group 1 | conjugated to tetanus toxoid carrier protein |
| | 20 μg/mL Neisseria meningitidis group W polysaccharide |
| | conjugated to tetanus toxoid carrier protein |
| | 20 μg/mL Neisseria meningitidis group Y polysaccharide |
| | conjugated to tetanus toxoid carrier protein |
| | (total tetanus toxoid carrier protein 110 μg/mL) |
| MenACWY + | 20 μg/mL Neisseria meningitidis group A polysaccharide |
| mod-AlPO₄ | conjugated to tetanus toxoid carrier protein |
| adjuvant | 20 μg/mL Neisseria meningitidis group C polysaccharide |
| (PZC 4.5) - | conjugated to tetanus toxoid carrier protein |
| Group 2 | 20 μg/mL Neisseria meningitidis group W polysaccharide |
| | conjugated to tetanus toxoid carrier protein |
| | 20 μg/mL Neisseria meningitidis group Y polysaccharide |
| | conjugated to tetanus toxoid carrier protein |
| | (total tetanus toxoid carrier protein 110 μg/mL) |
| | 0.8 mg/ml of mod-AlPO₄ adjuvant |
| MenPenta | 100 μg/mL of fHBP A05 tmN |
| (MenACWY + | 100 μg/mL of fHBP B01smN |
| MenB) + | 100 μg/mL of NadA |
| mod-AlPO₄ | 250 μg/mL of dOMV |
| adjuvant | 20 μg/mL Neisseria meningitidis group A polysaccharide |
| (PZC 4.5) - | conjugated to tetanus toxoid carrier protein |
| Group 3 | 20 μg/mL Neisseria meningitidis group C polysaccharide |
| | conjugated to tetanus toxoid carrier protein |
| | 20 μg/mL Neisseria meningitidis group W polysaccharide |
| | conjugated to tetanus toxoid carrier protein |
| | 20 μg/mL Neisseria meningitidis group Y polysaccharide |
| | conjugated to tetanus toxoid carrier protein |
| | (total tetanus toxoid carrier protein 110 μg/mL) |
| | 0.8 mg/ml of mod-AlPO₄ adjuvant |

### Dosing Regimen

Three groups (1, 2 and 3) of six rabbits each (female; strain: NZW KBL; 9-10 weeks old on D0) were used. Group 1 received the MenACWY immunogenic composition without AlPO₄ and groups 2 and 3 received, respectively, an MenACWY immunogenic composition with 400 μg of mod-AlPO₄ (PZC 4.5) adjuvant either without or with MenB immunogenic composition. These formulations were administered via IM injection on D0 and D28 (500 μL in the right thigh for the first injection and 500 μL in the left thigh for the second injection). Blood samples were collected under local anesthesia at the median artery of the rabbit's ear on D0, D28 and D42 (two weeks after the last injection on D42 for all groups).

TABLE 5
DOSING REGIMEN

| | | | Route / | |
|---|---|---|---|---|
| | Antigens | | Volume | Animal / |
| Group | quantity /dose | Adjuvant | injected | group |
| 1 | 10 μg Neisseria meningitidis group | None | IM - | 6 |
| | A polysaccharide conjugated to | | 500 μL | |
| | tetanus toxoid carrier protein | | | |
| | 10 μg Neisseria meningitidis group C | | | |
| | polysaccharide conjugated to tetanus | | | |
| | toxoid carrier protein | | | |
| | 10 μg Neisseria meningitidis group | | | |
| | W polysaccharide conjugated to | | | |
| | tetanus toxoid carrier protein | | | |
| | 10 μg/mL Neisseria meningitidis | | | |
| | group Y polysaccharide conjugated | | | |
| | to tetanus toxoid carrier protein | | | |
| | (total tetanus toxoid carrier protein | | | |
| | 55 μg/mL) | | | |
| 2 | 10 μg Neisseria meningitidis group | mod-AlPO₄ | IM - | 6 |
| | A polysaccharide conjugated to | (PZC 4.5) | 500 μL | |
| | tetanus toxoid carrier protein | | | |
| | 10 μg Neisseria meningitidis group C | | | |
| | polysaccharide conjugated to tetanus | | | |
| | toxoid carrier protein | | | |
| | 10 μg Neisseria meningitidis group | | | |
| | W polysaccharide conjugated to | | | |
| | tetanus toxoid carrier protein | | | |
| | 10 μg/mL Neisseria meningitidis | | | |
| | group Y polysaccharide conjugated | | | |
| | to tetanus toxoid carrier protein | | | |
| | (total tetanus toxoid carrier protein | | | |
| | 55 μg/mL) | | | |
| 3 | 10 μg Neisseria meningitidis group | mod-AlPO₄ | IM - | 6 |
| | A polysaccharide conjugated to | (PZC 4.5) | 500 μL | |
| | tetanus toxoid carrier protein | | | |
| | 10 μg Neisseria meningitidis group C | | | |
| | polysaccharide conjugated to tetanus | | | |
| | toxoid carrier protein | | | |
| | 10 μg Neisseria meningitidis group | | | |
| | W polysaccharide conjugated to | | | |
| | tetanus toxoid carrier protein | | | |
| | 10 μg/mL Neisseria meningitidis | | | |
| | group Y polysaccharide conjugated | | | |
| | to tetanus toxoid carrier protein | | | |
| | (total tetanus toxoid carrier protein | | | |
| | 55 μg/mL) | | | |
| | 50 μg of fHBP A05 tmN | | | |
| | 50 μg of fHBP B01smN | | | |
| | 50 μg mL of NadA | | | |
| | 125 μg of dOMV | | | |

Biological Sampling and hSBA

### Biological Sampling

Biological sampling was carried out on D0, D28 and D42 according to the procedure described above for the evaluation of the immunogenicity MenB immunogenic compositions.

hSBA for MenB Antigens

hSBA for MenB antigens was carried out as described above for the evaluation of the immunogenicity MenB immunogenic compositions.

hSBA for MenACWY Antigens
IgG Purification of Rabbit Sera for hSBA Testing

To avoid non-specific bactericidal killing induced by rabbit sera collected on D0 and D42, a purification of IgG was necessary.

Purification of rabbit sera was performed using rProtein A GravtiTrap™ columns (GE healthcare GE28-9852-54) and Ab Buffer Kit GE Healthycare ref 28-9030-59).

First, the columns were equilibrated with binding buffer (phosphate sodium 20 mM pH=7). After adjustment of pH sera to 7 with binding buffer (V/V), the sera were added to the column to perform IgG binding. The column was washed with binding buffer. Elution buffer (glycine HCl 0.1M pH2.7) was then added to the column to collect IgG. To preserve activity of IgG, neutralizing buffer (Tris-HCl 1M, pH9.0) was added to the elution fraction to obtain a final pH of approximatively 7. Quantification of IgG concentration was done by Nanodrop.

### Serum Bactericidal Activity

The bactericidal titers of individual purified sera (purified IgG) from immunized rabbits were measured by in vitro quantification of antibody-dependent complement mediated killing of N. meningitidis serogroups A, C, W135 or Y. The SBA assay measures the ability of antibodies to lyse bacteria in the presence of complement.

The source of complement was a human complement (Ig-depleted human serum). Briefly, sera were heat-inactivated 30 min at 56° C. and subsequently serially two-fold diluted (9 times) in Dulbecco PBS buffer containing Ca²⁺/Mg²⁺, 0.1% dextrose and 0.5% bovine serum albumin (dilution buffer) in a 96-well microplate.

Precultures of bacteria were grown on PVX medium plates (Chocolate agar+PolyViteX) at +37° C. in 5% CO₂ for 15 h for serogroup Y or for 18 h for serogroups A, C and W-135 to obtain isolated colonies. From overnight plates, bacteria were spread onto fresh PVX medium plates to obtain a light veil of confluent bacterial growth after 4 h at +37° C. in 5% CO₂. After incubation, bacteria were diluted to obtain 8·10³ CFU/mL. 50 μL of pre-diluted sera, 25 μL of human complement and 25 μL of bacterial working suspension were deposited in a 96-well microplate and incubated at +37° C. with shaking (100 rpm) for 60 min for serogroups C, W-135 and Y or for 90 min for serogroup A. After the appropriate incubation time, 50 μL of each well were transferred in a flat bottom plate and 100 μL of TSB agar were added to all wells. Plates were incubated at +37° C. with 5% CO₂ for 6-8 hours.

After incubation, the number of colonies per well was counted by using Cytation 7 equipment and Gen5 software (Biotek).

The bactericidal titer was defined as the dilution of the test serum that resulted in at least a 50% decrease in colony forming units (CFUs) per mL of bacteria compared to complement control well. Analyses were performed using Softmax Pro v6.5.1 GXP integrated in Sanofi Universal Exporter (SUE) and by selecting Gen5 WARP module.

### Data Analysis

A 2-way analysis of Variance (ANOVA) with time, product and their interaction as fixed factors was performed. Data were paired on time.

### Measure of Antigens Stability in MenB, MenACWY and MenPenta (MenB+MenACWY) Immunogenic Compositions

### Measure of Antigenicity of the MenB Antigens

Measures of antigens stability under thermal stress was carried out by measure of antigenicity of the MenB antigens (A05tm, B01sm, NadA and dOMV) and free polysaccharides of serogroup A, C, W and Y in MenPenta immunogenic composition incubated under thermal stress (45° C., or 37° C. for NadA) as a function of time.

A direct enzyme-linked immunosorbent assay (ELISA) was used to determine the relative antigenicity of the Neisseria meningitidis serogroup B antigens (B01smN, A05tmN, and NadA) in the MenPenta immunogenic compositions.

Briefly, 96-well microtiter plates were coated with samples of the immunogenic compositions (except for NadA—see below) obtained according to the thermal stress protocol and diluted in carbonate-bicarbonate buffer at an optimized starting antigen concentration. The samples were serially diluted with 8 points of serial dilutions on the plate and incubated overnight at 2° C. to 8° C. The following day, the plate was washed 3 times with wash buffer (PBS 1×+0.1% Tween-20) followed by incubation for 1 hour at room temperature with a specific monoclonal detection antibody (B01smN: JAR5 mAb and A05tmN: JAR13 mAb, both provided by from Children's Hospital Oakland Research Institute (CHORI); dOMV: P1.2 mAb obtained from National Institute for Biological Standards and Control (NIBSC)) conjugated to horseradish peroxidase (HRP).

In the case of NadA, 96-well microtiter plates were coated with an in-house anti-NadA specific monoclonal antibody, diluted in carbonate-bicarbonate buffer and incubated overnight at 2° C. to 8° C. The following day, the plate was washed 3 times with wash buffer (PBS 1×+0.1% Tween-20) followed by a 45-minute blocking step. Following blocking, samples of the immunogenic compositions obtained according to the thermal stress protocol were serially diluted with 8 points of serial dilutions on the plate and incubated for 2 hours at room temperature. The plate was washed and incubated for 1 hour at room temperature with a second in-house NadA specific monoclonal detection antibody conjugated to HRP.

Following the plate washing step, the plate was developed using 3,3′,5,5′-tetramethylbenzidine (TMB) as a substrate.

The reaction was stopped after 15 minutes (12 minutes for NadA) with 2N sulfuric acid (H₂SO₄) and the plate was read using a spectrophotometer. Color development was quantified by measuring the absorbance of each well at a wavelength of 450 nm with a reference wavelength of 540 nm. The magnitude of the colour development is directly proportional to the concentration of the antigen captured from the MenPenta immunogenic composition sample. The relative antigenicity (RA) of the samples was calculated using the SoftMax Pro software by comparing to the reference standard lot. Relative antigenicity is the reportable value for this assay.

### Measure of Free Polysaccharide Changes for Seroproups A, C, W-135 and Y

The free polysaccharide was measured by (high-performance anion-exchange chromatography/pulsed amperometric detection (HPAEC-PAD), as above indicated.

### Example 2: Time of Onset of Sedimentation (Tₒₙₛₑₜ)

The time of onset of sedimentation (Tₒₙₛₑₜ) is related to flocculation properties of a suspension. A Tₒₙₛₑₜ, above 60 minutes, suggests a high level of deflocculation, which could result in denser cake and poor cake resuspension properties of formulations. AlPO₄ formulations with Tₒₙₛₑₜ below 20 minutes are reported to be well flocculated, exhibiting better cake forming properties (Muthurania, 2015).

It was found that the MenB and MenPenta immunogenic compositions formulated with a mod-AlPO₄ adjuvant with a PZC of 4.5, in 50 mM sodium acetate, 150 mM NaCl, and pH 6.0 showed better suspension properties with longer sedimentation onset time compared to the formulations made with AlPO₄ of PZC above 5.

Although, all formulations had a Tₒₙₛₑₜ below 20 minutes suggesting well flocculated formulations, the increased Tₒₙₛₑₜ by the reduction of the PZC of AlPO₄ adjuvant has several advantages including better adjuvant and formulation mixing and reconstitution, which in turn, decreases the complexity of the filling operation process. A longer Tₒₙₛₑₜ supports ease of handling of the product at the clinic, which allows to maintain homogeneity for longer hold times between mixing, withdrawal into syringe and injection.

TABLE 6
TIME OF ONSET OF SEDIMENTATION OF MENB
AND MENPENTA FORMULATIONS COMPRISING
AlPO₄ ADJUVANT OR MOD-AlPO₄ ADJUVANT

| FORMULATION | Tₒₙₛₑₜ (min) |
|---|---|
| MENPENTA + AlPO₄ adjuvant (PZC 5.2) | 1.5 |
| MENPENTA + mod-AlPO₄ adjuvant (PZC 4.5) | 4.5 |
| MENB + AlPO₄ adjuvant (PZC 5.2) | 3.0 |
| MENB + mod-AlPO₄ adjuvant (PZC 4.5) | 8.5 |

### Example 3: Pyrogenicity of dOMV Formulated with Mod-AlPO₄ Adjuvant Vs AlPO₄ Adjuvant

MenB with AlPO₄ PZC 5.2 or with mod-AlPO₄ PZC 4.5 were prepared as above disclosed. Pyrogenicity of the compositions was evaluated as indicated in the above.

A rise in temperature and pyrogenicity is known to be associated with the dOMV components. Adsorption to an aluminum adjuvant is known to reduce the pyrogenicity of dOMV (Rosenqvist, 1998).

The dOMV adsorption was almost 100% irrespective of the PZC of AlPO₄ (see example 7). The dOMV adsorption onto mod-AlPO₄ with a PZC of about 4.5 showed similar IL-6 EC₅₀ values compared to dOMV adsorbed on AlPO₄ adjuvant, irrespective of the dOMV dose used (high dose of 250 μg/mL or low dose of 50 μg/mL). These results reveal no impact on the pyrogenicity due to the modification of the PZC of AlPO₄ adjuvant.

TABLE 7
EC₅₀ VALUES FOR dOMV WHEN ADSORBED TO
TWO AlPO₄ (NON-MODIFIED AND MODIFIED
PZC) AT TWO DIFFERENT dOMV DOSES

EC₅₀ (ng/ml)

| FORMULATION | Low dose | High dose |
|---|---|---|
| dOMV + AlPO₄ adjuvant | 13 | 12 |
| dOMV + mod-AlPO₄ adjuvant | 15 | 13 |

### Example 4: Serum Bactericidal Activity of MenB Immunogenic Compositions

hSBA Response for fHBPs

Bactericidal activity was measured in IgG purified from individual sera collected from all immunized rabbits on D0 and D42, using human IgG/IgM depleted complement at a final concentration of 15%.

hSBA Against Closely-Related fHBP A56 and Heterologous fHBP A22 Strains

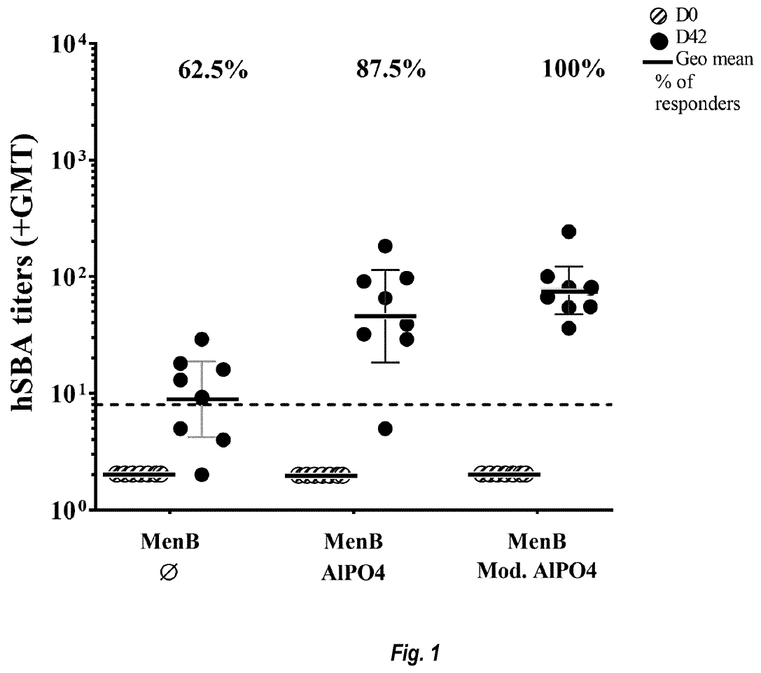

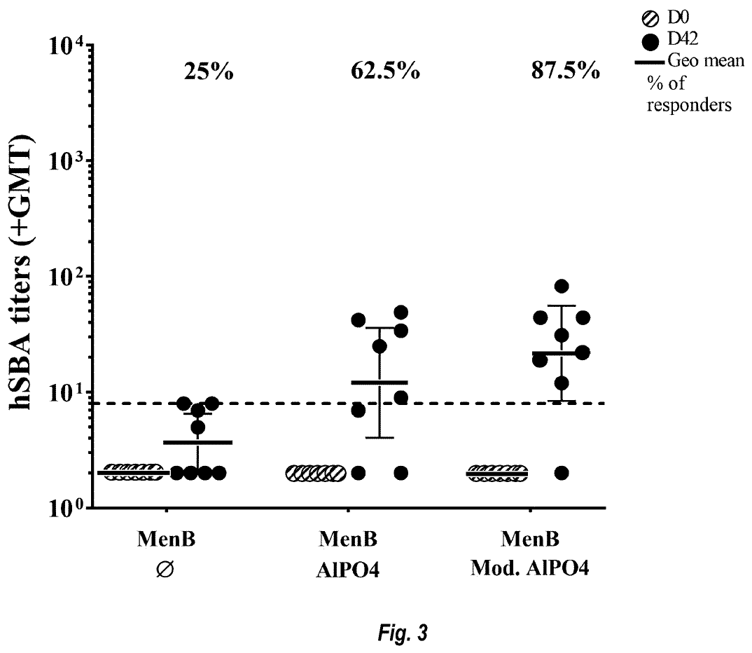

As depicted in FIGS. 1 and 3, without AlPO₄ adjuvant, A05 tmN of MenB immunogenic composition was able to induce a moderate fHBP-specific hSBA response against both close-related variant fHBP A (A56; FIG. 1) strains with 62.5 of responders and geometric mean titers (GMT) of 9, and a low fHBP-specific hSBA response against heterologous variant fHBP A (A22; FIG. 3) strain with 25% of responders and a GMT of 4.

Further, as shown on FIGS. 1 and 3, the presence of AlPO₄ adjuvant in MenB immunogenic composition increased the A05 tmN-induced hSBA responses against the close-related A56 strain by 5.1 (p-value=0.001)-fold (GMT of 46) and against the heterologous A22 strain by 2.8 (p-value=0.049)-fold (GMT of 12), compared to the control (composition without adjuvant), while the mod-AlPO₄ adjuvant further increased the response by 8.4 (p-value<0.001)-fold (GMT of 76) against the close-related A56 strain and by 4.9 (p-value=0.004)-fold (GMT of 22) against the heterologous A22 strain.

Finally, the AlPO₄ adjuvant induced a % of responders of 87.5 against the close-related A56 strain and of 62.5% against the heterologous strain A22, while the mod-AlPO₄ adjuvant induced a % of responders of 100% against the close-related A56 strain and of 87.5% against the heterologous strain A22.

The results are summarized in the following Table 8:

TABLE 8

| | closely related - fHBP A56 | | | heterologous - fHBP A22 | | |
|---|---|---|---|---|---|---|
| | Comp. A | Compo. B | Comp. C | Comp. A | Compo. B | Comp. C |
| Parameter | (control) | (+AlPO₄) | (+modAlPO₄) | (control) | (+AlPO₄) | (+modAlPO₄) |
| GMT | 9 | 46 | 76 | 4 | 12 | 22 |
| hSBA fold | — | 5.1 | 8.4 | — | 2.8 | 4.9 |
| % | 62.5 | 87.5 | 100 | 25 | 62.5 | 87.5 |
| responders | | | | | | |

As observed in FIGS. 1 and 3, higher and more homogeneous hSBA responses (GMT and number of responders) were observed against fH-BP A with the formulation comprising mod-AlPO₄ adjuvant, but with no statistical difference between mod-AlPO₄ adjuvant and AlPO₄ adjuvant.

hSBA Against Closely-Related fHBP B44 Strains and Heterologous fHBP B24 Strains

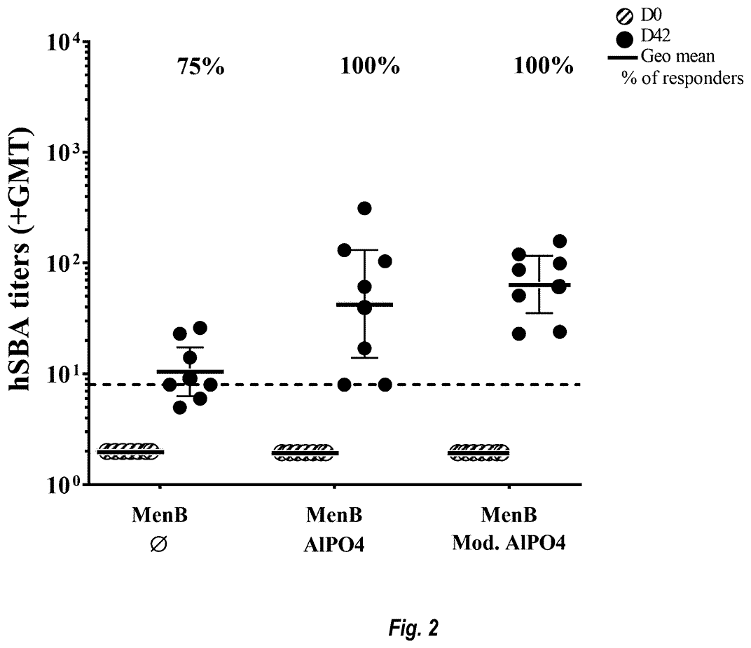

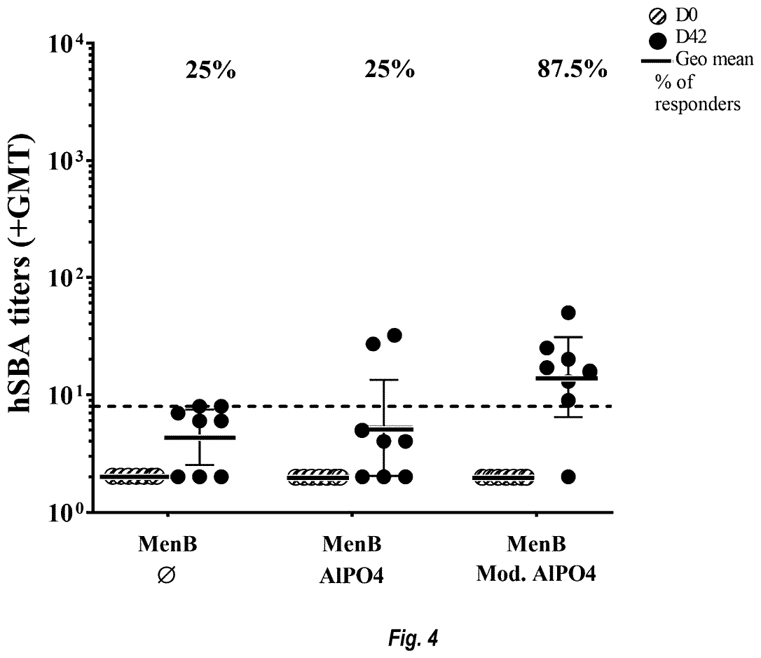

As depicted in FIGS. 2 and 4, without AlPO₄ adjuvant, B01smN of MenB immunogenic composition was able to induce a moderate fHBP-specific hSBA response against close-related variant fHBP B (B44; FIG. 2) strains with 75% of responders and geometric mean titers (GMT) of 10, and a low fH-BP-specific hSBA response against heterologous variant fHBP B (B24; FIG. 4) strain with 25% of responders and a GMT of 4.

Further, as shown on FIGS. 2 and 4, the presence of AlPO₄ adjuvant in MenB immunogenic composition increased the B01 smN-induced hSBA responses against the close-related B44 strain by 3.8 (p-value=0.011)-fold (GMT of 43) while it did not induce an increase of the response against the heterologous B24 strain (GMT of 5), compared to the control (composition without adjuvant). By contrast, the mod-AlPO₄ adjuvant further increased the response by 6.2 (p-value=0.001)-fold (GMT of 64) against the close-related B44 strain and by 3.2 (p-value=0.019)-fold (GMT of 14) against the heterologous B24 strain.

The AlPO₄ adjuvant induced a % of responders of 100% against the close-related B44 strain and of 25% against the heterologous strain B24, while the mod-AlPO₄ adjuvant induced a % of responders of 100% against the close-related B44 strain and of 87.5% against the heterologous strain B24.

The results are summarized in the following Table 9:

TABLE 9

| | closely-related - fHBP B44 | | | heterologous - fHBP B24 | | |
|---|---|---|---|---|---|---|
| | Comp. A | Compo. B | Comp. C | Comp. A | Compo. B | Comp. C |
| Parameter | (control) | (+AlPO₄) | (+modAlPO₄) | (control) | (+AlPO₄) | (+modAlPO₄) |
| GMT | 10 | 43 | 64 | 4 | 5 | 14 |
| hSBA fold | — | 5.1 | 6.2 | — | 0.8 | 3.2 |
| % | 75 | 100 | 100 | 25 | 25 | 87.5 |
| responder | | | | | | |

As observed in FIGS. 2 and 4, higher hSBA responses were observed against fH-BP B with the formulation comprising mod-AlPO₄ adjuvant, and with statistical difference with AlPO₄ adjuvant for variant B24 (p-value=0.043).

hSBA Response for dOMV and NadA
hSBA Against the Homologous VR2-P1.2-PorA Strain (dOMV Response)

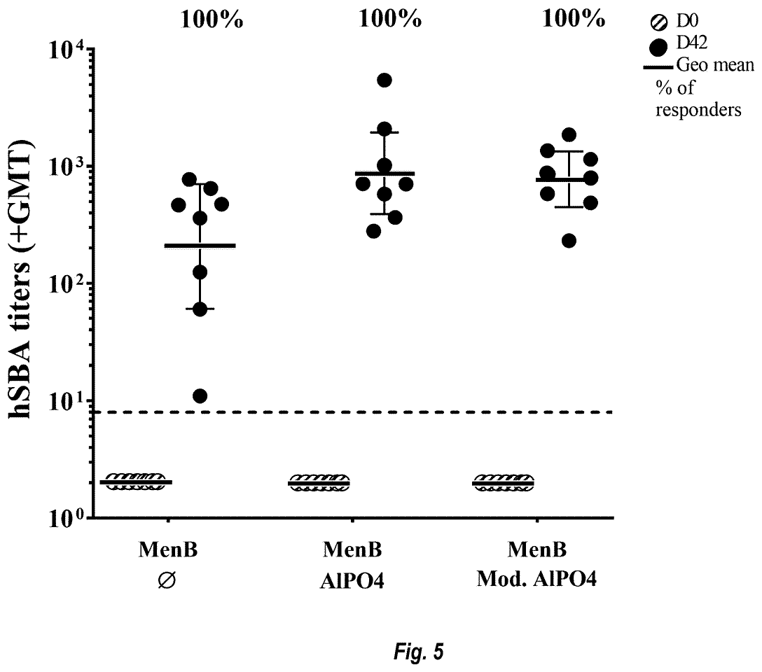

As depicted in FIG. 5, without adjuvant, dOMV was able to induce a high hSBA response against the homologous PorA VR2 P1.2 strain with 100% of responders and with a GMT of 206.

The presence of AlPO₄ adjuvant or mod-AlPO₄ adjuvant to MenB immunogenic composition increased significantly the dOMV-induced hSBA responses against the homologous VR2-P1.2-PorA strain with 4.9 (p-value=0.007) and 4 (p-value=0.015)-fold increase, GMT of 875 and 775 and % of responders of 100%, respectively.

No statistical difference was observed between AlPO₄ adjuvant or mod-AlPO₄ adjuvant responses.

hSBA Against Homologous NadA Strain

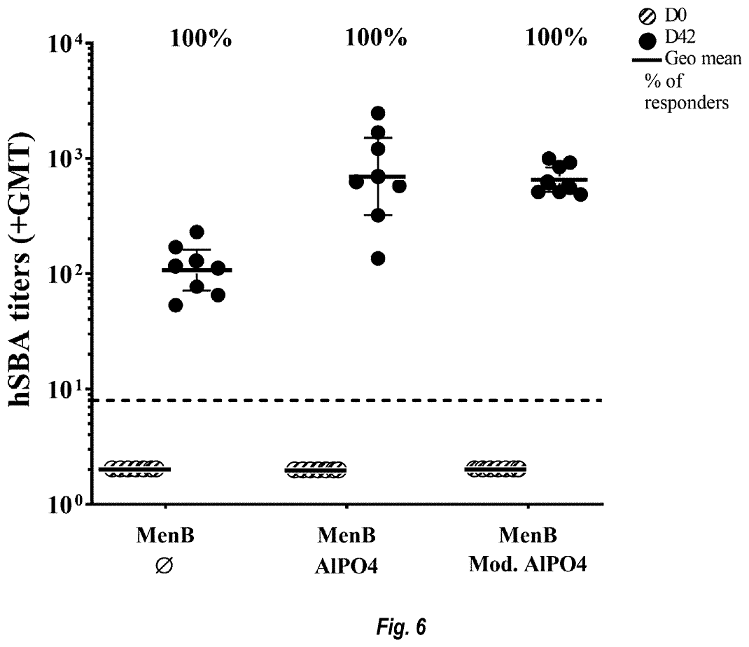

As depicted in FIG. 6, without AlPO₄ adjuvant, NadA was able to induce a high hSBA response against the homologous NadA1 strain with 100% of responders with a GMT of 107.

The presence of AlPO₄ adjuvant or mod-AlPO₄ adjuvant to MenB immunogenic increased significantly the NadA-induced hSBA responses against the homologous NadA strain with 6.4 (p-value<0.001) and 5.7 (p-value<0.001)-fold increase, GMT of 698 and 652 and % of responders of 100%, respectively.

No statistical difference was observed between AlPO₄ adjuvant or mod-AlPO₄ adjuvant responses.

### Conclusion

The objective of this study was to compare in the New-Zealand (NZ) white rabbits the immunogenicity of the MenB immunogenic composition (A05tmN+B01smN+NadA+dOMV) formulated or not with non-modified AlPO₄ adjuvant (PZC 5.2) or with phosphate-modified AlPO₄ (PZC 4.5) based on hSBA response.

The MenB immunogenic composition formulated without AlPO₄ was able to induce bactericidal activity with % of responders ranging from 25 to 100%. When formulated in presence of either AlPO₄ or mod-AlPO₄ adjuvant, the MenB immunogenic composition was able to induce significantly higher hSBA titers (all p-value ≤0.049) compared to the group without AlPO₄, with 2.8-to-8.4-fold increase depending on the strain used. For the B24 strain a significant higher response compared to formulation with AlPO₄ was observed in presence of mod-AlPO₄ adjuvant (p-value=0.019, fold increase of 3.2). And overall, a higher and more homogeneous hSBA responses induced by A05tmN and B01smN fHBPs were observed in presence of mod-AlPO₄ adjuvant. In term of % of responders and geometric mean titers (GMT) with depending on the strain used:
87.5 to 100% with MenB composition with mod-AlPO₄ adjuvant compared to 25 to 100% with MenB composition with AlPO₄ adjuvant.
GMT ranging of 14 to 76 in mod-AlPO₄ adjuvant compared to 5 to 46 in AlPO₄ adjuvant.

### Example 5: Serum Bactericidal Activity of MenACWY, with or without Mod-AlPO₄ Adjuvant (PZC 4.5), and of MenPenta Immunogenic Compositions

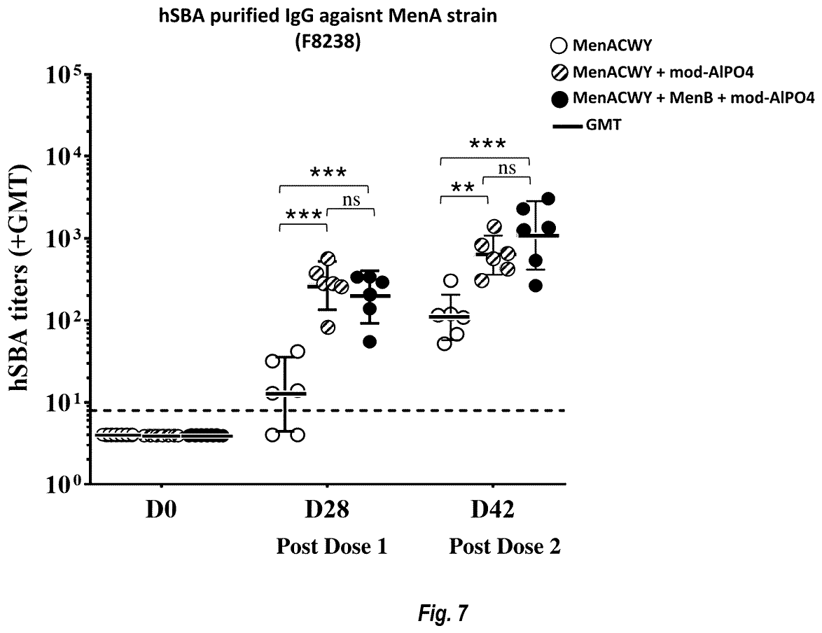

As show on FIGS. 7-10, the adsorption of MenACWY immunogenic composition onto mod-AlPO₄ adjuvant (PZC 4.5) did not negatively impact the hSBA against ACWY strains post dose 1 and 2.

For MenACWY immunogenic composition, 2 doses are needed to induce potent hSBA response in rabbits. Increase of the hSBA of MenACWY immunogenic composition in presence of mod-AlPO₄ adjuvant (PZC 4.5) was observed on D28 and D42.

As shown on FIG. 7, the presence of mod-AlPO₄ adjuvant (PZC 4.5) in MenACWY immunogenic composition increased the hSBA against A strain post dose 1 and 2. Compared to MenACWY alone, a significant increase of hSBA titers against A strain in presence of mod-AlPO₄ adjuvant was observed (on D28 p<0.001 ×21.2; on D42 p<0.001 ×5.8). Also, a higher significant hSBA response with the MenPenta (MenACWY+MenB) immunogenic composition formulated with AlPO₄ adjuvant was observed (on D28 p<0.001 ×15.4; on D42 p<0.001 ×10.1).

Compared to MenACWY+mod-AlPO₄ adjuvant, no significant impact of addition of MenB antigens (MenACWY+mod-AIPO4+MenB) on the hSBA titers against A strain was observed.

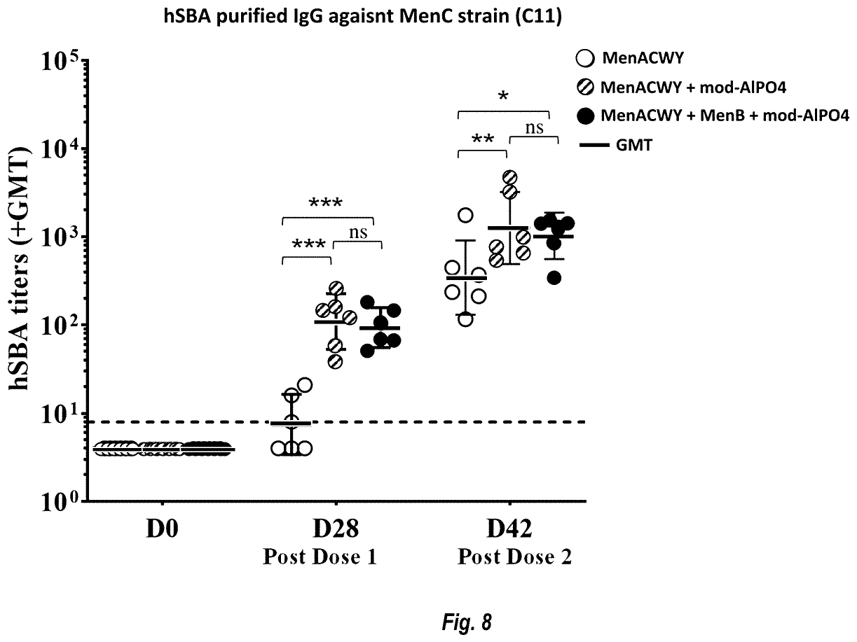

As shown on FIG. 8, the presence of mod-AlPO₄ adjuvant (PZC 4.5) in MenACWY immunogenic composition significantly increased the hSBA against C strain post dose 1 and 2. Compared to MenACWY alone, a significant increase of hSBA titers against C strain in presence of mod-AlPO₄ adjuvant was observed (on D28 p<0.001 ×14.6; on D42 p=0.008 ×3.6). Also, a higher significant hSBA response with the MenPenta (MenACWY+MenB) immunogenic composition formulated with AlPO₄ adjuvant was observed (on D28 p<0.001 ×12.5; on D42 p<0.022 ×3).

Compared to MenACWY+mod-AlPO₄ adjuvant, no significant impact of addition of MenB antigens (MenACWY+mod-AIPO₄+MenB) on the hSBA titers against C strain was observed.

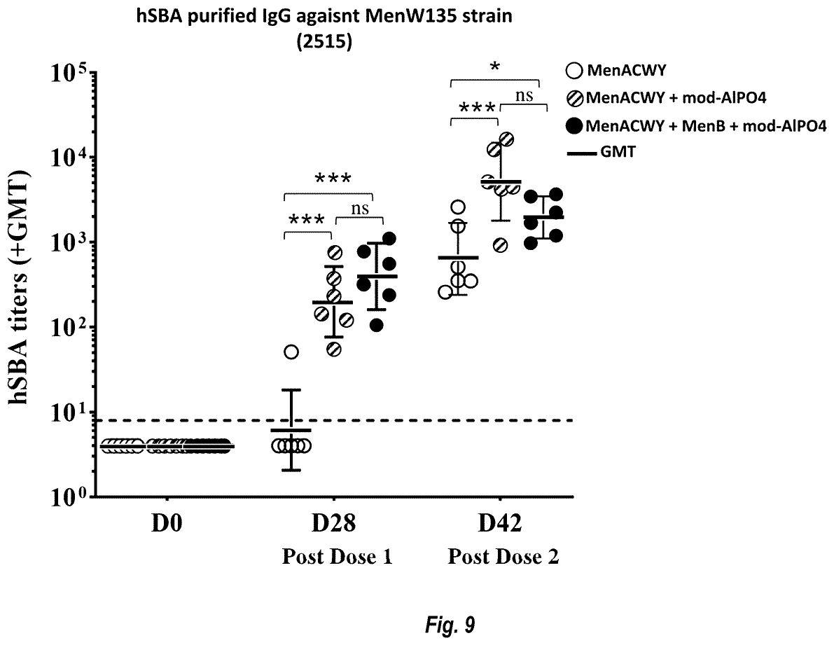

As shown on FIG. 9, the presence of mod-AlPO₄ adjuvant (PZC 4.5) in MenACWY immunogenic composition increased the hSBA against W strain post dose 1 and 2. Compared to MenACWY alone, a significant increase of hSBA titers against W strain in presence of mod-AlPO₄ adjuvant was observed (on D28 p<0.001 ×32.4; on D42 p=0.001 ×8.1). Also, a higher significant hSBA response with the MenPenta (MenACWY+MenB) immunogenic composition formulated with AlPO₄ adjuvant was observed (on D28 p<0.001 ×64.3; on D42 p<0.022 ×3.1).

Compared to MenACWY+mod-AlPO₄ adjuvant, no significant impact of addition of MenB antigens (MenACWY+mod-AIPO4+MenB) on the hSBA titers against W strain was observed.

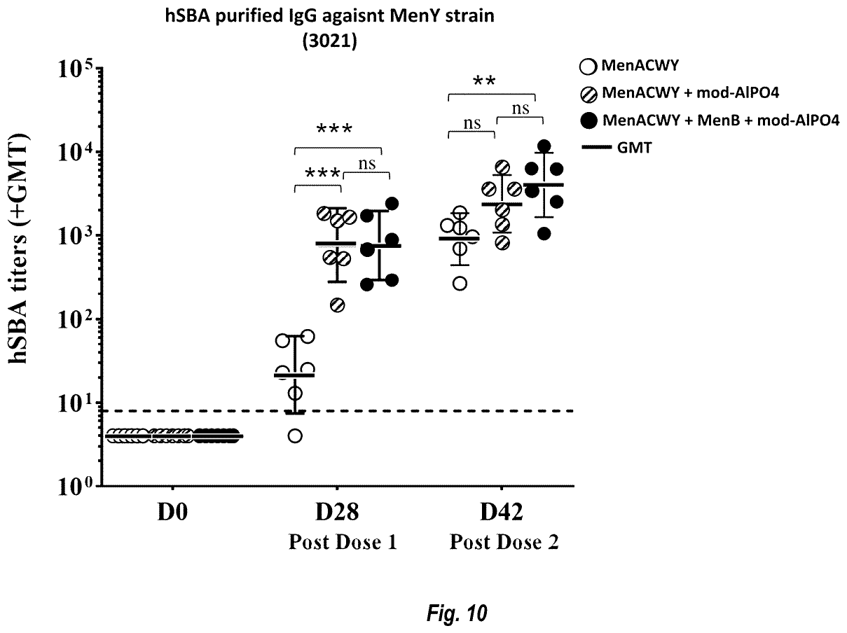

As shown on FIG. 10, the presence of mod-AlPO₄ adjuvant (PZC 4.5) in MenACWY immunogenic composition increased the hSBA against Y strain post dose 1. Compared to MenACWY alone, a significant increase of hSBA titers against Y strain in presence of mod-AlPO₄ adjuvant was observed (on D28 p<0.001 ×35.4; on D42 not significant). Also, a higher significant hSBA response with the MenPenta (MenACWY+MenB) immunogenic composition formulated with AlPO₄ adjuvant was observed (on D28 p<0.001 ×35; on D42 p=0.01 ×4.4).

Compared to MenACWY+mod-AlPO₄ adjuvant, no significant impact of addition of MenB antigens (MenACWY+mod-AIPO4+MenB) on the hSBA titers against Y strain was observed.

### Example 6: Antigen Stability in MenB and MenPenta Immunogenic Compositions

MenB Antigens (A05tm, B01sm, NadA and dOMV)

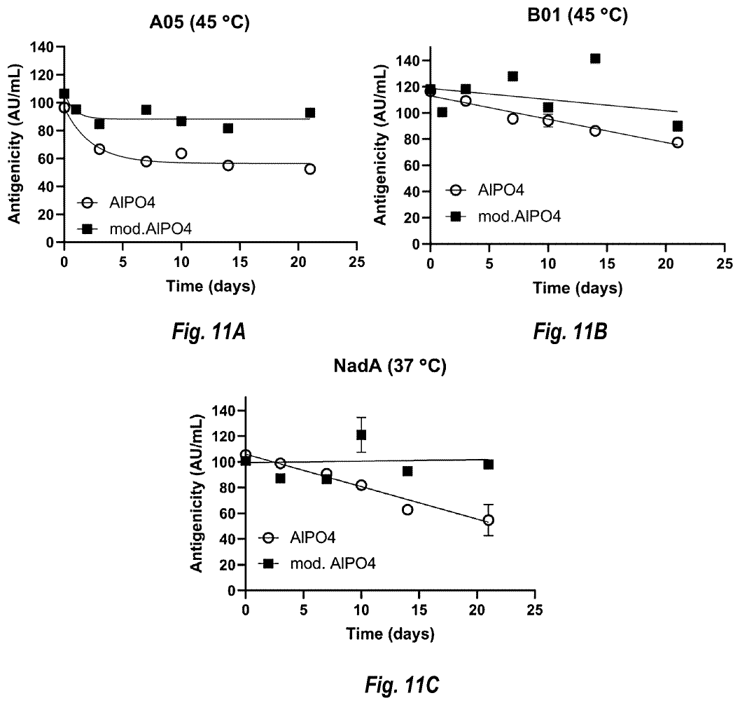

The stability of the individual antigens in MenB formulations was assessed in AlPO₄ or mod.AlPO₄ at 37° C. (NadA) and 45° C. (B01smN, A05tmN, and dOMV) by ELISA. The results indicated that A05tmN was significantly more stable when formulated in mod.AlPO₄ compared to AlPO₄ (p=0.0003). NadA was also found to be significantly more stable in mod.AlPO₄ compared to AlPO₄ (p=0.0082). A slight improvement of stability is shown for B01smN formulated in mod.AlPO₄, but no significant differences were detected among B01smN formulated in mod.AlPO₄ vs. AlPO₄ (FIGS. 11A-11C). No difference was detected among dOMV formulated in mod.AlPO₄ vs. AlPO₄ (result not shown).

This shows that the MenB immunogenic composition has better antigenicity/potency over time under accelerated temperature conditions, which results in an improved shelf life.

### Free Polysaccharide of a, C, W and Y Serogroup in MenACWY or MenPenta Immunogenic Compositions

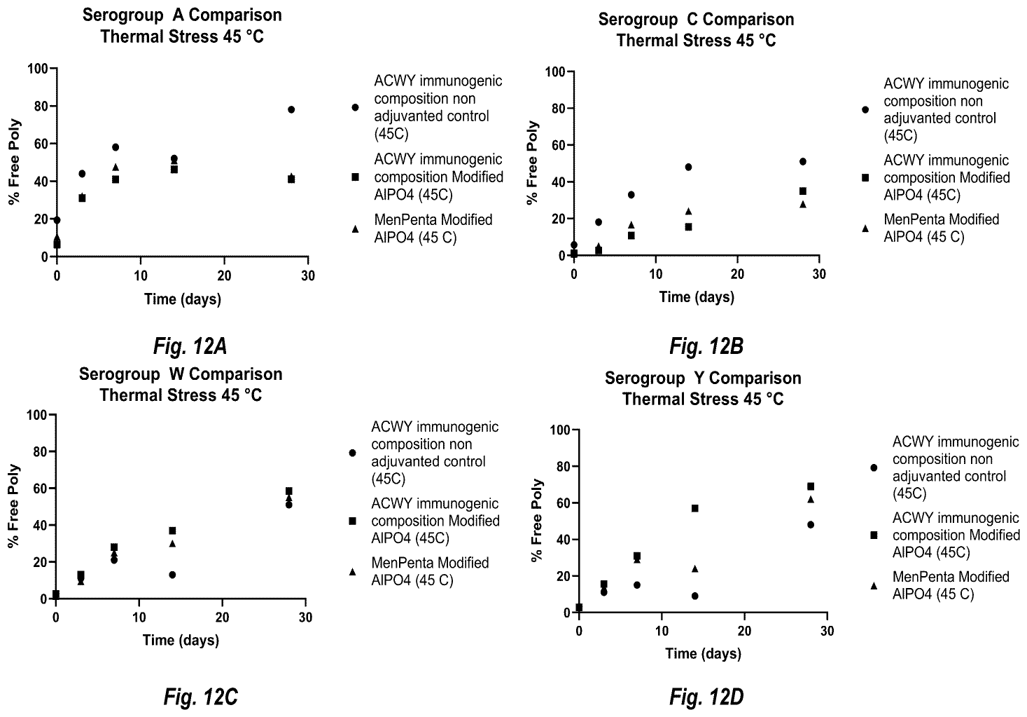

As shown on FIGS. 12A-12D, no impact of AlPO₄ adjuvant PZC modification on stability (monitored by percent free polysaccharide content) of Serogroup A, C, W-135 and Y conjugates was observed in MenACWY or MenPenta compositions compared to non-adsorbed serogroup A, C, W and Y (MenACWY without adjuvant) at accelerated thermal stress of 45° C.

Non-adsorbed A, C, W-135 and Y conjugates are stable up to 4 years at 2-8° C. thus suggesting a good long term stability for A, C, W-135 and Y. Similar degradation profiles under accelerated thermal stress for non-adsorbed A, C, W-135, Y to the one adsorbed to AlPO₄ with modified PZC (4.5) in presence of fHBPs, NadA and dOMV suggest a good long term stability of serogroup A, C, W-135 and Y in a MenPenta immunogenic composition (FIGS. 12A-12D).

### Example 7: Antigens Adsorption on AlPO₄

The adsorption of the MenB antigens on AlPO₄ was assayed in compositions, MenB or MenPenta, with AlPO₄ with various PZC (5.2 or 4.5-mod.AlPO₄) and pH 6. The results are presented in the Table 8.

TABLE 8
PERCENTAGE OF MENB ANTIGENS ADSORBED ON
AlPO₄ (NON-MODIFIED AND MODIFIED PZC)

| | % Adsorption | | | |
|---|---|---|---|---|
| | | | | dOMV (based |
| Formulation | A05tmN | B01smN | NadA | on PorB) |
| MenB PZC 5.2 | 88 | 82 | 99 | 99 |
| MenB PZC 4.5 | 74 | 73 | 7 | 99 |
| MenPenta PZC 5.2 | 88 | 83 | 99 | 99 |
| MenPenta PZC 4.5 | 71 | 70 | 7 | 92 |

### Example 8: Antigen Stability in MenB Immunogenic Compositions

The stability of the antigens in MenB immunogenic compositions (A05tm, B01sm, NadA and dOMV) formulated with a mod-AlPO₄ adjuvant with a PZC of 4.3, 4.5, 4.8, and an AlPO₄ adjuvant with a PZC 5.2, in 50 mM sodium acetate, 150 mM NaCl, pH 6.0 was assessed at 37° C. (NadA) and 45° C. (B01smN, A05tmN, and dOMV) for 30 days. The stability was evaluated by sandwich ELISA measure of antigenicity of the MenB antigens according to the following protocol:

96-well microtiter plates were coated with an in-house anti-fHBP A05, an in-house anti-fHBP B01, an in-house anti-NadA specific monoclonal antibody or, for dOMV, an anti-Porin B monoclonal antibody (from NIBSC), and incubated overnight between 2° C. to 8° C. The following day, the plates were washed 3 times with a wash buffer followed by a blocking step. Following blocking, samples obtained according to the thermal stress protocol were serially diluted with 8 points of serial dilutions on the plate and incubated for 2 hours at room temperature. The plates were washed and incubated for 1 hour at room temperature with a detection monoclonal antibody conjugated to HRP consisting of a second in-house anti-fHBP A05 monoclonal antibody, a second in-house anti-fHBP B01 monoclonal antibody, a second in-house anti-NadA monoclonal antibody or, for dOMV, an anti-Porin A monoclonal antibody (from NIBSC).

Following the plates washing step, the plates were developed using 3,3′,5,5′-tetramethylbenzidine (TMB) as a substrate.

The reaction was stopped with 2N sulfuric acid (H₂SO₄) after the appropriate incubation time and the plates were read using a spectrophotometer. Color development was quantified by measuring the absorbance of each well at a wavelength of 450 nm with a reference wavelength of 540 nm. The magnitude of the color development is directly proportional to the concentration of the antigen captured from the MenB immunogenic composition sample.

The data was analyzed using SoftMax Pro GxP v6.5.1 software. An equivalence approach is to assess parallelism between the reference standard and positive control and between the reference standard and each test sample. The Parallel Line Analysis (PLA) module available within the SMP software was used to determine the relative antigenicity (reported as relative potency in SoftMax Pro) of the positive control and each test sample. The relative potency value determined for a sample was used to generate a reportable value of “Antigenicity Units per mL” (AU/mL) based on arbitrary unit conversion relative to the reference standard.”

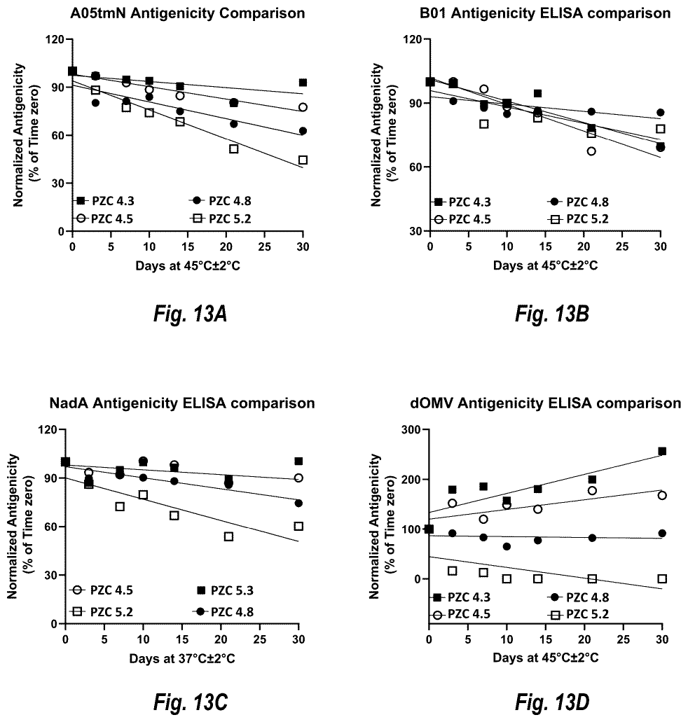

As can be noted from FIGS. 13A-13D, it was observed that:
A05tmN was significantly more stable in formulations containing AlPO₄ with PZC ≤4.8 (ANCOVA: p<0.0001).
NadA was significantly more stable in formulations containing AlPO₄ with PZC ≤4.5 (ANCOVA: p=0.023).
dOMV was significantly more stable in formulations containing AlPO₄ with PZC ≤4.5 (ANCOVA: p<0.0169).

No significant difference was observed for B01sm among the different formulations.

In conclusion, the results indicated that A05tmN was significantly more stable when formulated in AlPO₄ with PZC≤4.8. NadA and dOMV were found to be significantly more stable in AlPO₄ with PZC ≤4.5. No significant differences were detected in the stability of B01smN formulated in AlPO₄ in the range of PZC between 4.3 and 5.2.

Altogether, this result shows that the MenB immunogenic composition formulated with a mod-AlPO₄ adjuvant with a PZC below 5.0 has a more stable antigenicity/potency over time under accelerated temperature conditions, which results in an improved shelf life.

### REFERENCES

Alving, C. R. (1993). Novel adjuvant strategies for experimental malaria and AIDS vaccines. Annals of the New York Academy of Sciences, 690, 265-275. doi:10.1111/j.1749-6632.1993.tb44015.x
Batista R S, Gomes A P, Dutra Gazineo J L, Balbino Miguel P S, Santana L A, Oliveira L, et al. Meningococcal disease, a clinical and epidemiological review. Asian Pac J Trop Med. 2017; 10(11):1019-29.
Bijlsma M W, Brouwer M C, Spanjaard L, van de Beek D, van der Ende A. A decade of herd protection after introduction of meningococcal serogroup C conjugate vaccination. Clin Infect Dis. 2014; 59(9):1216-21.
Bruce M G, Rosenstein N E, Capparella J M, Shutt K A, Perkins B A, Collins M. Risk factors for meningococcal disease in college students. JAMA. 2001; 286(6):688-93.
Campsall P A, Laupland K B, Niven D J. Severe meningococcal infection: a review of epidemiology, diagnosis, and management. Crit Care Clin. 2013; 29(3):393-409.
Caron F, du Chatelet I P, Leroy J P, Ruckly C, Blanchard M, Bohic N, et al. From tailor-made to ready-to-wear meningococcal B vaccines: longitudinal study of a clonal meningococcal B outbreak. Lancet Infect Dis. 2011; 11(6):455-63.
Christensen H, May M, Bowen L, Hickman M, Trotter C L. Meningococcal carriage by age: a systematic review and meta-analysis. Lancet Infect Dis. 2010; 10(12):853-61.
Diminsky, D. M. (1999). Physical, chemical and immunological stability of CHO-derived hepatitis B surface antigen (HBsAg) particles. Vaccine, 18(1-2), 3-17. doi:10.1016/s0264-410x(99)00149-8
Dyet K H, Martin D R. Clonal analysis of the serogroup B meningococci causing New Zealand's epidemic. Epidemiol Infect. 2006; 134(2):377-83.
Folaranmi T, Rubin L, Martin S W, Patel M, MacNeil J R, Centers for Disease C. Use of Serogroup B Meningococcal Vaccines in Persons Aged >/=10 Years at Increased Risk for Serogroup B Meningococcal Disease: Recommendations of the Advisory Committee on Immunization Practices, 2015. MMWR Morb Mortal Wkly Rep. 2015; 64(22):608-12.
Fredriksen J H, Rosenqvist E, Wedege E, Bryn K, Bjune G, Froholm L O, et al. Production, characterization and control of MenB-vaccine “Folkehelsa”: an outer membrane vesicle vaccine against group B meningococcal disease. NIPH Ann. 1991; 14(2):67-79; discussion-80.
Germinario C, Tafuri S, Napoli C, Montagna M T, Balducci M T, Fortunato F, et al. Young-adult carriers of Neisseria meningitidis in Puglia (Italy): will the pattern of circulating meningococci change following the introduction of meningococcal serogroup C conjugate vaccines?Hum Vaccin. 2010; 6(12):1025-7.
Grodet C, Dequin P F, Watt S, Lanotte P, de Gialluly C, Taha M K, et al. Outbreak in France of Neisseria meningitidis B:15:P1.12 belonging to sequence type 1403. Clin Microbiol Infect. 2004; 10(9):845-8.
Harrison L, Granoff D, Pollard A. Meningococcal capsular group A, C, W, and Y conjugate vaccines. [ed.] Orenstein W A, Offit P A, Edwards K M Plotkin S A. Vaccines. 7. Philadelphia (PA): Elsevier; 2018. p. 619-43.
Harrison O B, Claus H, Jiang Y, Bennett J S, Bratcher H B, Jolley K A, et al. Description and nomenclature of Neisseria meningitidis capsule locus. Emerg Infect Dis. 2013; 19(4):566-73.
Hem, S. L. (2007). Imject Alum is not aluminum hydroxide adjuvant or aluminum phosphate adjuvant. Vaccine, 25(27), 4985-4986. doi:10.1016/j.vaccine.2007.04.078
Hem, S. L. (2007). Relationship between physical and chemical properties of aluminum-containing adjuvants and immunopotentiation. Expert Rev Vaccines, 6(5), 685-698. doi:10.1586/14760584.6.5.685
Iyer, S. R. (2004). Mechanism of adsorption of hepatitis B surface antigen by aluminum hydroxide adjuvant. Vaccine, 22(11-12), 1475-1479. doi:10.1016/j.vaccine.2003.10.023
Jones, L. S. (2005). Effects of adsorption to aluminum salt adjuvants on the structure and stability of model protein antigens. The Journal of biological chemistry, 280(14), 13406-13414. doi:10.1074/jbc.M500687200 Kvalsvig A J, Unsworth D J. The immunopathogenesis of meningococcal disease. J Clin Pathol. 2003; 56(6):417-22.
Maa, Y. F. (2003). Stabilization of alum-adjuvanted vaccine dry powder formulations: mechanism and application. Journal of pharmaceutical sciences, 92(2), 319-332. doi:10.1002/jps.10294
MacLennan J, Kafatos G, Neal K, Andrews N, Cameron J C, Roberts R, et al. Social behavior and meningococcal carriage in British teenagers. Emerg Infect Dis. 2006; 12(6):950-7.
Maiden M C, Ibarz-Pavon A B, Urwin R, Gray S J, Andrews N J, Clarke S C, et al. Impact of meningococcal serogroup C conjugate vaccines on carriage and herd immunity. J Infect Dis. 2008; 197(5):737-43.
Muthurania, K. I. (2015). Investigation of the Sedimentation Behavior of Aluminum Phosphate: Influence of pH, Ionic Strength, and Model Antigens. Journal of pharmaceutical sciences, 104(11), 3770-3781. doi:10.1002/jps.24584
Pace D, Pollard A J. Meningococcal disease: clinical presentation and sequelae. Vaccine. 2012; 30 Suppl 2:B3-9.
Rinella, J. V. (1996). Treatment of aluminium hydroxide adjuvant to optimize the adsorption of basic proteins. Vaccine, 14(4), 298-300. doi:10.1016/0264-410x(95)00194-6
Rodriguez A P, Dickinson F, Baly A, Martinez R. The epidemiological impact of antimeningococcal B vaccination in Cuba. Mem Inst Oswaldo Cruz. 1999; 94(4):433-40.
Rosenqvist, E. H. (1998). Effect of aluminium hydroxide and meningococcal serogroup C capsular polysaccharide on the immunogenicity and reactogenicity of a group B Neisseria meningitidis outer membrane vesicle vaccine. Developments in biological standardization, 92, 323-333.
Rouphael N G, Stephens D S. Neisseria meningitidis: biology, microbiology, and epidemiology. Methods Mol Biol. 2012; 799:1-20.
Seeber, S. J. (1991). Predicting the adsorption of proteins by aluminium-containing adjuvants. Vaccine, 9(3), 201-203. doi:10.1016/0264-410x(91)90154-x
Stephens D S, Apicella M A. Neisseria meningitidis. [ed.]J. E. Bennett, R. Dolin and M. J. Blaser. Philadelphia: Elsevier Saunders; 2015. p. 2425-45.
Stephens D S. Biology and pathogenesis of the evolutionarily successful, obligate human bacterium Neisseria meningitidis. Vaccine. 2009; 27 Suppl 2:B71-7.
Trotter C L, Andrews N J, Kaczmarski E B, Miller E, Ramsay M E. Effectiveness of meningococcal serogroup C conjugate vaccine 4 years after introduction. Lancet. 2004; 364(9431):365-7.
Vuocolo S, Balmer P, Gruber W C, Jansen K U, Anderson A S, Perez J L, et al. Vaccination strategies for the prevention of meningococcal disease. Hum Vaccin Immunother. 2018; 14(5):1203-15.
Warren, H. S. (1986). Current status of immunological adjuvants. 4, 369-388. doi:10.1146/annurev.iy.04.040186.002101
Zheng, Y. L. (2007). The structural stability of protein antigens adsorbed by aluminium hydroxide in comparison to the antigens in solutions. Spectroscopy, 21(5-6), 257-268.

1. An immunogenic composition comprising a combination of Neisseria meningitidis serogroup B antigens, said combination comprising at least one factor H binding protein (fHBP) A and at least one factor H binding protein (fHBP) B, and an aluminum hydroxyphosphate (AlPO₄) adjuvant, the AlPO₄ adjuvant being selected as having a point of zero charge (PZC) below 5.

2. The immunogenic composition according to claim 1, wherein the AlPO₄ adjuvant is selected to have a PZC ranging from about 4.1 to less than 5, or ranging from about 4.2 to about 4.9, or ranging from about 4.3 to about 4.8, or to be about 4.5.

3. The immunogenic compositions according to claim 1, wherein the difference between the PZC of the AlPO₄ adjuvant and a pH of the composition is ranging from about 0.6 to about 2.9.

4. The immunogenic composition according to claim 1, wherein the composition has a pH ranging from about 5.5 to about 7.0 or being about 6.0.

5. The immunogenic composition according to claim 1, further comprising at least one detergent-extracted Outer Membrane Vesicle (dOMV), in particular present in an amount ranging from about 5 μg/dose to about 400 μg/dose, or from about 10 μg/dose to about 300 μg/dose, or from about 25 μg/dose to about 250 μg/dose, or from about 35 μg/dose to about 225 μg/dose, or from about 50 μg/dose to about 200 μg/dose, or from about 75 μg/dose to about 180 μg/dose, or from about 100 μg/dose to about 150 μg/dose, or from about 110 μg/dose to about 125 μg/dose, or at about 25 μg/dose, or at about 50 μg/dose, or at about 125 μg/dose,
and/or at least one Neisseria adhesin A (NadA) protein, in particular present in an amount ranging from about 20 μg/dose to about 200 μg/dose, or from about 25 μg/dose to about 180 μg/dose, or from about 40 μg/dose to about 140 μg/dose, or from about 50 μg/dose to about 120 μg/dose, or from about 75 μg/dose to about 100 μg/dose, or at about 50 μg/dose.

6. The immunogenic composition according to claim 1, wherein the fHBPs are adsorbed onto AlPO₄ at an amount of 85%, or less, of the total amount of fHBPs of the composition, or at an amount ranging from about 50% to less than 85% of the total amount of fHBPs of the composition.

7. The immunogenic composition according to claim 1, wherein the fHBP B has an isoelectric point (pI) ranging from about 5.0 to about 7.0, or from 5.2 to about 6.5, or from about 5.3 to about 6, or is about 5.46 and/or wherein the fHBP A has an isoelectric point (pI) ranging from about 5 to about 7, or from 5.2 to about 6.5, or from about 5.4 to about 6, or is about 5.86.

8. The immunogenic composition according to claim 1, wherein the fHBP B and/or the fHBP A is non-lipidated.

9. The immunogenic composition according to claim 1, wherein the fHBP B is a mutated fHBP B comprising at least one mutation reducing or suppressing the binding of the fHBP B to the human factor H (fH), and/or is a mutated fHBP B comprising at least about 85% identity with SEQ ID NO: 3, and/or is a mutated fHBP B comprising at least one amino acid substitution selected from at least one of: a) an amino acid substitution of the glutamine at amino acid 38 (Q38); b) an amino acid substitution of the glutamic acid at amino acid 92 (E92); c) an amino acid substitution of the arginine at amino acid 130 (R130); d) an amino acid substitution of the serine at amino acid 223 (S223); and e) an amino acid substitution of the histidine at amino acid 248 (H₂₄₈), based on the numbering of SEQ ID NO:6, and/or comprises or consists of SEQ ID NO: 4, and/or comprises or consists of SEQ ID NO: 9.

10.-13. (canceled)

14. The immunogenic composition according claim 1, wherein the fHBP A is a mutated fHBP A comprising at least one mutation reducing or suppressing the binding of the fHBP A to the human factor H (fH), and/or is a mutated protein comprising at least about 85% identity with SEQ ID NO: 1, and/or is a mutated fHBP A comprising at least one amino acid substitution selected from at least one of: a) an amino acid substitution of the asparagine at amino acid 115 (N115); b) an amino acid substitution of the aspartic acid at amino acid 121 (D121); c) an amino acid substitution of the serine at amino acid 128 (S128); d) an amino acid substitution of the phenylalanine at amino acid 129 (F129); e) an amino acid substitution of the leucine at amino acid 130 (L130); f) an amino acid substitution of the valine at position 131 (V131); g) an amino acid substitution of the glycine at position 133 (G133); h) an amino acid substitution of the lysine at position 219 (K219); and i) an amino acid substitution of the glycine at position 220 (G220), based on the numbering of SEQ ID NO:6, and/or comprises or consists of SEQ ID NO: 2, and/or comprises or consists of SEQ ID NO: 8.

15.-16. (canceled)

17. The immunogenic composition according to claim 1, wherein the fHBP A and/or the fHBP B are each present in an amount ranging from about 20 μg/dose to about 200 μg/dose, or from about 25 μg/dose to about 180 μg/dose, or from about 40 μg/dose to about 140 μg/dose, or from about 50 μg/dose to about 120 μg/dose, or from about 75 μg/dose to about 100 μg/dose, or at an amount of about 50 μg/dose, or about 100 μg/dose.

18. The immunogenic composition according to claim 5, wherein the NadA protein is NadA1 protein, or comprises at least about 85% identity with SEQ ID NO: 5 or comprises or consists of SEQ ID NO:5.

19. (canceled)

20. The immunogenic composition according to claim 5, wherein the dOMV comprises a porin A (PorA) protein, in particular comprises a porin A (PorA) protein selected among PorA VR2 subtypes or is a PorA VR2 P1.2.

21.-22. (canceled)

23. The immunogenic composition according to claim 1, wherein the composition further comprises a buffer, in particular a buffer selected among a Tris buffer, an acetate buffer, a citrate buffer, a phosphate buffer, an HEPES buffer, or a histidine buffer, more particularly the buffer is a sodium acetate buffer.

24.-25. (canceled)

26. The immunogenic composition according to claim 1, comprising or consisting of 25 to 100 μg/dose of a non-lipidated mutated fHBP A consisting of SEQ ID NO: 2 or of a non-lipidated mutated fHBP A consisting of SEQ ID NO: 8, 25 to 100 μg/dose of a non-lipidated mutated fHBP B consisting of SEQ ID NO: 4 or of a non-lipidated mutated fHBP B consisting of SEQ ID NO: 9, 25 to 100 μg/dose of a NadA protein consisting of SEQ ID NO: 5, 20 to 250 μg/dose of dOMV from a MenB strain expressing PorA VR2 P1.2, 100 to 800 μg/dose of an AlPO₄ adjuvant selected as having a PZC of about 4.5, 50 mM of acetate buffer and pH 6.0

27. The immunogenic composition according to claim 1, further comprising at least a capsular saccharide from one or more of Neisseria meningitidis serogroups A, C, W135 and/or Y conjugated to a carrier protein, in particular wherein the conjugated capsular saccharide is conjugated to a tetanus toxoid carrier.

28.-31. (canceled)

32. A vaccine comprising an immunogenic composition according to claim 1.

33. A method for inducing an immune response against a Neisseria meningitidis B strain, comprising administering the immunogenic composition according to claim 1 to a subject.

34. A method for enhancing an immune response induced by a composition comprising a N. meningitidis fHBP B antigen against a N. meningitidis serogroup B strain expressing a fHBP B antigen heterologous to said fHBP B antigen of said composition, or
for stabilizing at least one of fHBP A and NadA protein in an immunogenic composition, or
for stabilizing a time of onset of sedimentation (Tₒₙₛₑₜ) of a composition comprising a combination of Neisseria meningitidis serogroup B antigens, said combination comprising at least one factor H binding protein (fHBP) A and at least one factor H binding protein (fHBP) B, in a range from about 3.5 min to about 10 min, or
for adjuvanting an immunogenic composition comprising a combination of Neisseria meningitidis serogroup B antigens, said combination comprising at least one factor H binding protein (fHBP) A and at least one factor H binding protein (fHBP) B, or
for manufacturing an immunogenic composition comprising a combination of Neisseria meningitidis serogroup B antigens, said combination comprising at least one factor H binding protein (fHBP) A and at least one factor H binding protein (fHBP) B,
wherein the method comprises administering an AlPO₄ adjuvant having a PZC below 5 to a subject.

35.-40. (canceled)

41. A method for manufacturing an immunogenic composition comprising a combination of Neisseria meningitidis serogroup B antigens, said combination comprising at least one factor H binding protein (fHBP) A and one factor H binding protein (fHBP) B, and an AlPO₄ adjuvant, the method comprising at least the steps of:
a) selecting an AlPO₄ adjuvant having a PZC below 5, and
b) combining the AlPO₄ adjuvant selected at step a) with at least one factor H binding protein (fHBP) A and at least one factor H binding protein (fHBP) B, the combination being carried out in any order.

42. A method for stabilizing at least one of fHBP A and NadA protein in an immunogenic composition, the method comprising at least the steps of:
a) selecting an AlPO₄ adjuvant having a PZC below 5, and
b) combining the AlPO₄ adjuvant selected at step a) with a fHBP A or NadA protein, and
c) obtaining an immunogenic composition in which said fHBP A or NadA protein is stabilized.

43. A method for preparing an immunogenic composition comprising a N. meningitidis fHBP B antigen, said composition inducing an enhanced immune response against a N. meningitidis serogroup B strain expressing a fHBP B heterologous to said fHBP B antigen of said composition, the method comprising at least the steps of:
a) selecting an AlPO₄ adjuvant having a PZC below 5, and
b) combining the AlPO₄ adjuvant selected at step a) with said fHBP B antigen, and
c) obtaining said immunogenic composition.

44. The method according to claim 34, wherein (1) the AlPO₄ adjuvant has a PZC ranging from about 4.1 to less than 5, or ranging from about 4.2 to about 4.9, or ranging from about 4.3 to about 4.8, or to be about 4.5; or (2) the difference between the PZC of the AlPO₄ adjuvant and a pH of the composition is ranging from about 0.6 to about 2.9.

45. The method according to claim 34, wherein the immunogenic composition is a combination of Neisseria meningitidis serogroup B antigens, said combination comprising at least one factor H binding protein (fHBP) A and at least one factor H binding protein (fHBP) B, and an aluminum hydroxyphosphate (AlPO₄) adjuvant, the AlPO₄ adjuvant being selected as having a point of zero charge (PZC) below 5.

46. A method for inducing an immune response against a Neisseria meningitidis serogroup B strain in an individual in need thereof, the method comprising at least a step of administering to said individual an immunogenic composition according to claim 1, wherein said step of administration induces an immune response against said Neisseria meningitidis serogroup B strain.

47. A method for enhancing an immune response induced by a composition comprising a N. meningitidis fHBP B antigen against a N. meningitidis serogroup B strain expressing a fHBP B heterologous to said fHBP B antigen of said composition, in an individual in need thereof, the method comprising at least a step of administering to said individual an immunogenic composition according to claim 1, wherein said step of administration induces an enhanced immune response against said Neisseria meningitidis serogroup B strain expressing said heterologous fHBP B.


SEQUENCE LISTING (9 sequences)

SEQ ID NO: 1 - protein, 261 aa, Neisseria meningitidis
  REGION 1..261: note = fHbp A05
CSSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 2 - protein, 261 aa, Neisseria meningitidis
  REGION 1..261: note = fHbp A05tmN
CSSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFRVSDLGGEHTAFNQLPSGKAEYHGKAFSSDDAGGKLTYTIDFAAKQGHGKIEHLKTPEQNVELASAELKADEKSHAVILGDTRYGSEEKSTYHLALFGDRAQEIAGSATVKIREKVHEIGIAGKQ

SEQ ID NO: 3 - protein, 260 aa, Neisseria meningitidis
  REGION 1..260: note = fHbp B01
CSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 4 - protein, 260 aa, Neisseria meningitidis
  REGION 1..260: note = fHbp B01smN
CSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHLIGLAAKQ

SEQ ID NO: 5 - protein, 284 aa, Neisseria meningitidis
  REGION 1..284: note = NadA1 C-terminally truncated
MTSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVG

SEQ ID NO: 6 - protein, 255 aa, Neisseria meningitidis
  REGION 1..255: note = fHbp B24
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 7 - protein, 362 aa, Neisseria meningitidis
  REGION 1..362: note = NadA1
MKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 8 - protein, 261 aa, Neisseria meningitidis
  source 1..261: note = non-lipidated fHbp A05tm
MSSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFRVSDLGGEHTAFNQLPSGKAEYHGKAFSSDDAGGKLTYTIDFAAKQGHGKIEHLKTPEQNVELASAELKADEKSHAVILGDTRYGSEEKSTYHLALFGDRAQEIAGSATVKIREKVHEIGIAGKQ

SEQ ID NO: 9 - protein, 260 aa, Neisseria meningitidis
  source 1..260: note = non-lipidated fHbp B01sm
MSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHLIGLAAKQ